(19) United States
(12) Patent Application Publication
(10) Pub. No.: US 2016/0272720 A1
(43) Pub. Date: Sep. 22, 2016

(54) Compositions and Methods for the Treatment of Acute Myeloid Leukemias and Myelodysplastic Syndromes

(71) Applicant: MEMORIAL SLOAN-KETTERING CANCER CENTER, New York, NY (US)

(72) Inventors: Christopher Y. Park, New York, NY (US); Stephen Shiu-Wah Chung, New York, NY (US)

(73) Assignee: Memorial Sloan-Kettering Cancer Center, New York, NY (US)

(21) Appl. No.: 15/035,217

(22) PCT Filed: Nov. 6, 2014
(86) PCT No.: PCT/US2014/064408

§ 371 Date: May 6, 2016

Related U.S. Application Data
(60) Provisional application No. 61/900,997, filed on Nov. 6, 2013.
(60) Provisional application No. 61/901,437, filed on Nov. 7, 2013.

(51) Int. Cl. C07K 16/28; A61K 39/395; A61K 45/06; C12Q 1/68

(52) U.S. Cl. CPC C07K 16/2896; C12Q 1/6886; A61K 39/39558; A61K 45/06; C12Q 2600/106; C12Q 2600/158; C07K 2317/734; C07K 2317/732

(57) ABSTRACT

Provided are compositions and methods for the treatment of hematological conditions, in particular CD99+ acute myelogenous leukemias (AML) and myelodysplastic syndromes (MDS), which comprise one or more antibody that (a) binds to the extracellular domain of CD99, (b) ligates AML and/or MDS cell-surface expressed CD99, (c) promotes the capping/clustering/aggregation AML and/or MDS cell-surface expressed CD99, and (d) induces apoptosis in and consequent cytotoxicity of antibody-ligated CD99+ AML and/or MDS cells. Disclosed methods include methods for identifying AML and MDS patients that are susceptible to treatment with an anti-CD99 antibody by detecting the elevated expression of CD99 in a tissue sample or cell from an AML or MDS patient and for treating an AML and/or MDS patient exhibiting elevated CD99 gene and or cell-surface protein expression by administering a composition comprising an anti-CD99 antibody, either alone or in combination with one or more additional component such as a mobilizing agent, a transmigration blocking agent, and an AML and/or MDS chemotherapeutic agent, such as daunorubicin, idarubicin, cytarabine, 5-azacytidine, and decitabine.

### CROSS-REFERENCE TO RELATED APPLICATIONS

This application is being filed on Nov. 6, 2014, as a PCT International Patent application and claims the benefit of U.S. Provisional Applications No. 61/900,997 filed Nov. 6, 2013 and No. 61/901,437 filed Nov. 7, 2013. Each such provisional application is incorporated by reference in its entirety.

### BACKGROUND OF THE DISCLOSURE

1. Technical Field

The present disclosure relates, generally, to the treatment of hematological conditions, in particular acute myeloid leukemias (AML) and myelodysplastic syndromes (MDS). More specifically, this disclosure concerns: (i) anti-CD99 antibodies, and compositions comprising one or more anti-CD99 antibody(ies), for the treatment of acute myeloid leukemias and the myelodysplastic syndromes; (ii) methods for generating and for identifying anti-CD99 antibodies that are suitable for the treatment of acute myeloid leukemia and/or a myelodysplastic syndrome; (iii) methods for identifying a patient having acute myeloid leukemia and/or the myelodysplastic syndrome that is susceptible to treatment with an anti-CD99 antibody and/or a compositions comprising one or more anti-CD99 antibody(ies); (iv) methods for inducing apoptosis in a CD99 cell that is associated with acute myeloid leukemia and/or the myelodysplastic syndrome, such as a CD99 leukemic stem cell and/or a hematopoietic stem cell; and (v) methods for the treatment of a CD99 acute myeloid leukemia and/or a CD99 myelodysplastic syndrome.

2. Description of the Related Art

Acute myeloid leukemia (AML), also known as acute myelogenous leukemia or acute nonlymphocytic leukemia (ANLL), is a cancer of the myeloid line of blood cells characterized by the rapid growth of abnormal white blood cells that accumulate in the bone marrow and interfere with the production of normal blood cells. AML is the most common acute leukemia. affecting adults, and its incidence increases with age. Although AML is a relatively rare disease, accounting for approximately 1.2% of cancer deaths in the United States, its incidence is expected to increase as the population ages.

Clinical signs and symptoms of AML are caused by replacement of normal bone marrow with leukemic cells, which causes a drop in red blood cells, platelets, and normal white blood cells (cytopenia). These signs and symptoms include fatigue, shortness of breath, easy bruising and bleeding, and increased risk of infection, splenomegaly or hepatomegaly and other symptoms caused by leukemic infiltration of tissues may also be present. Several risk factors, chromosomal abnormalities, and other somatic mutations have been identified, but the specific cause is not clear. As an acute leukemia, AML progresses rapidly and is typically fatal within weeks or months if left untreated.

AML has several subtypes; treatment and prognosis varies among subtypes. Five-year survival varies from 15-70%, and relapse rate varies from 33-78%, depending on subtype. AML is treated initially with chemotherapy aimed at inducing a remission; patients may go on to receive additional chemotherapy or an allogeneic hematopoietic stem cell transplant.

The treatment for patients with acute myelogenous leukemia (AML) has not changed in over 20 years, and AML survival rates remain significantly below 50% for adults and around 60-70% for children. Even if patients are cured of their disease, there is often significant morbidity from conventional chemotherapy regimens and from bone marrow transplantation. More effective, less toxic therapies are clearly needed.

The myelodysplastic syndromes (MDS, formerly known as preleukemia) are a diverse collection of hematological (blood-related) medical conditions that involve ineffective production (and dysplasia) of the myeloid lineage of blood cells. Patients with MDS can develop severe anemia. and require blood transfusions. In some cases, the disease worsens and the patient develops cytopenias (low blood counts) caused by progressive bone marrow failure.

The prognosis in MDS depends on the type and severity. Many people live normal life-spans with MDS. Often, people are asymptomatic and are unaware they even have MDS until it shows up in a routine blood test.

The myelodysplastic syndromes are all disorders that arise in the hematopoietic stem cell in the bone marrow. In MDS, hetnatopoiesis (blood production) is disordered and ineffective. The number and quality of blood-forming cells decline irreversibly, further impairing blood production.

The myelodysplastic syndromes (MDS) represent a related group of clonal hematologic disorders characterized by peripheral cytopenias due to ineffective hematopoiesis. The syndromes may arise de 110VO, or secondarily after treatment with chemotherapy and/or radiation therapy for other diseases. Secondary mvelodysplasia usually has a poorer prognosis than does de novo myelodysplasia. MDS transforms to acute myeloid leukemia (AML) in about 30% of patients after various intervals from diagnosis.

MDS occurs predominantly in older patients, though patients as young as two years of age have been reported. Anemia, bleeding, easy bruising, and fatigue are common initial findings. Splenomegaly or hepatosplenomegaly may occasionally be present in association with an overlapping myeloproliferative disorder. Approximately 50% of the patients have a detectable cytogenetic abnormality, most commonly a deletion of all or part of chromosome 5 or 7, or trisomy 8. Although the bone marrow is usually hypercellular at diagnosis, 15% to 20% of patients present with a hypoplastic bone marrow. Hypoplastic myelodysplastic patients tend to have profound cytopenias and may respond more frequently to immunosuppressive therapy.

AML and MDS are both initiated and sustained by self-renewing stem cells. Lapidot et al., Nature 367:645-648 (1994); Bonnet and Dick, Nat. Med. 3:730-737 (1997); Nilsson et al., Blood 100:259-267 (2002); Nilsson et al., Blood 110:3005-3014 (2007); Tehranchi et al., New Engl. J. Med. 363:1025-1037 (2010); Nilsson et al., Blood 96:2012-2021 (2000); and Pang et of, Proc. Natl. Acad. Sci. U.S.A. 110:3011-3016 (2013). Although these disease initiating cells share immunophenotypic features of normal hematopoietic stem and progenitors (HSPCs), aberrant expression of cell surface proteins may allow for prospective isolation of disease stem cells and represent attractive therapeutic targets. The identification of CD99 as a cell surface protein that is highly expressed in AML leukemic stem cells (LSCs) and MDS hematopoietic stem cells (HSCs) is described. High CD99 expression is associated with disease aggressiveness in AML xenografts. In contrast, patients with high CD99 transcript expression have an improved prognosis, which may be due to increased chemosensitivity conferred by enhanced leukemic cell transendothelial migration and mobilization into the peripheral blood (PB). Finally, monoclonal antibody (mAb) based targeting of CD99 induces direct cytotoxicity in the absence of immune effector cells or complement, with relative sparing of normal HSCs and endothelial cells. Together, these data establish CD99 as a cell surface protein preferentially expressed in AML and MDS stem cells, as a mediator of transendothelial migration and mobilization of leukemic blasts, and as a promising therapeutic target for direct targeting by mAbs.

### SUMMARY OF THE DISCLOSURE

The present disclosure provides compositions and methods for the treatment of hematological conditions, in particular acute myeloid leukemia (AML) and the myelodysplastic syndromes (MDS). As is described in detail herein, AML and MDS may be effectively treated with one or more compound(s) that promotes the aggregation of cellular CD99. In particular, the present disclosure provides compositions comprising one or more anti-CD99 antibodies, wherein each anti-CD99 antibody: (i) binds to the extracellular domain of CD99; (ii) promotes the aggregation, clustering, and/or capping of CD99; and (iii) induces cell death in the AML and/or MDS cell to which the anti-CD99 antibody binds. Moreover, as evidenced herein, the induction of cell death correlates very closely with the combination of binding and one or more of aggregation, clustering or capping, such that the latter can serve to predict that cell death will occur.

Within one embodiment, the present disclosure provides anti-CD99 antibodies, and compositions comprising one or more anti-CD99 antibodies, for the treatment of acute myc.doid leukemias and the myelodysplastic syndromes.

Within another embodiment, the present disclosure provides methods for generating and for identifying anti-CD99 antibodies that are suitable for the treatment of acute myeloid leukemia andlor the myelodysplastic syndrome.

Within a further embodiment, the present disclosure provides methods for identifYing a patient having an acute myeloid leukemia and or a myelodysplastic syndrome that is susceptible to treatment with an anti-CD99 antibody and/or a composition comprising one or more anti-CD99 antibodies.

Within another embodiment, the present disclosure provides methods for inducit cell death in a CD99⁺ cell that is associated with an acute myeloid leukemia and/or a myelodysplastic syndrome, such as a CD99 leukemic stem cell and or a hematopoietic stem cell.

Tithin yet other embodiments, the present disclosure provides methods for the treatment of a CD99⁺ acute myeloid leukemia and/or a CD99⁺ myelodysplastic syndrome which exhibits elevated levels of CD99.

### BRIEF DESCRIPTION OF THE DRAWINGS

FIG. 1A is a bar graph showing that ligation of CD99 on MDS cell line MDS92 (Tohyama et al., Br. J. Haematol. 91:795 (1995)) with 20 μg/ml of anti-CD99 antibody designated 12E7 (Levy el al., Proc. Natl. Acad. Sci. U.S.A 76:6552 (1979)) is cytotoxic to those MDS92 cells as evidenced by a 128-fold decrease in MDS92 cell number (p<0.001) at 72 hours as compared to N1DS92 cell number in the presence of 20 μg/ml of an isotype control antibody. FIG. 1B is a plot of 7-AAD vs. Annexin V fluorescence values, whiCh demonstrates effector cell-independent apoptosis (as evidenced by a 77% increase in annexin V positivity (p<0.001.) of MDS92 cells following 72 hour ligation of CD99 with 10 μg/ml of 121E7 as compared to the absence of apoptosis of MDS92 cells following a 72-hour incubation in the presence of 20 μg/ml of an isotype control antibody.

FIG. 2A is a bar graph showing 121E7-mediated cytotoxicity as evidenced by a time-dependent 128-fold decrease in MDS92 cell number at 22 hours (p<0.001) following ligation of CD99 with anti-CD99 antibody 1.2E7 as compared to MDS92 cell number in the presence of an isotype control antibody. FIG. 2B is a log plot showing a time-dependent decrease in CD99 cell-surface expression on MDS92 cells following ligation of CD99 with anti-CD99 antibody 12E7. FIG. 2C is a plot of myeloid differentiation marker CD11b vs. CD14 fluorescence values, which demonstrates a time-dependent decrease in cell-surface expression of both markers following ligation of CD99 with anti-CD99 antibody 12E7.

FIG, 3 is a bar graph showing that ligation of CD99 on primary CD34 MDS cells with the 12E7 anti-CD99 antibody is cytotoxic to those primary MDS cells as evidenced by a 10-fold decrease in cell number relative to no antibody and an 8-fold decrease in cell number relative to isotype (IgG) control antibody after 48 hours.

FIG. 4A is a bar graph showing that ligation of CD99 on high-expressing CD99′ AML cell lines EIL60 and MOLM13 with the 12E7 anti-CD99 antibody is cytotoxic as evidenced by a 49-fold decrease (HL60; p<0.001) and a 70-fold decrease (MOLM13; p<0.001) in cell number relative to isotype control antibody after 72 hours. FIG. 4B is a log plot showing elevated levels of CD99 expression on MOLM13 and HL60 cells compared with isotype control antibody.

FIG. 5A is a bar graph showing that ligation of CD99 on CD99-expressing primary AML 1520 blast cells with 12E7 anti-CD99 antibody is cytotoxic as evidenced by a 57-fold decrease (p<0.001) in cell number after 48 hours. FIG. 5B is a bar graph showing that ligation of CD99 on CD99-expressing primary AML 890 blast cells with 12E7 anti-CD99 antibody is cytotoxic as evidenced by a 48-fold decrease (p<0.001) in cell number after 48 hours. FIG. 5C is a bar graph showing that 12E7 anti-CD99 antibody has only a modest cytotoxic effect on normal cord blood HSC as evidenced by a 1.4-fold decrease in HSC cell number after 80 hours.

FIGS. 6A-6F demonstrate that CD99 is overexpressed on disease-initiating stem cells in MDS and AML. FIG. 6A presents the results of flow-cytometrie (FC) analysis of hematopoietic stem cells (HSCs, lineage-negative (LN) CD34⁺CD38⁻CD90⁺D45RA⁻) from patients with therapy-related myelodysplastic syndrome (MDS) (n=26) and normal cord blood (CB) controls (n=27) were analyzed by flow-cytometry WC) for expression of CD99. FIG. 6B is a plot of unfractionated AML blasts (CD19−CD3−CD4510-svSSCio-sv)(n=63) and normal CB controls that were analyzed by PC for CD99 expression. Error bars represent±SD. Asterisk represents p<0.0001 (unpaired t-test). FIG. 6C presents the results of CD99 expression that was evaluated by flow cytometry in CD3−CD19−CD34+CD38− cells from the AML specimen UPenn 1956. By immunophenotype, “CD99 low” cells resembled predominantly HSCs and multipotent progenitors (MPPs, LN CD34+CD38−CD90−CD45RA−), while “CD99 high” resembled lymphoid-primed MPPs (LMPPs, LN CD34+CD38−CD90−CD45RA+). FIG. 6D is a bar graph showing that when “CD99 low” and “CD99 high” cell populations were double-sorted to >95% purity by PACS and plated in methylcellulose assays (1600 cells), a larger number of normal myeloid colonies were formed from the “CD99 low” fraction at 14 day⁻s, but not from the “CD99 high” population. FIG. 6E is an agarose gel showing that colonies derived from “CD99 low” cells lacked the FL 73-IID molecular abnormality present in the UPenn 1956 AML specimen (B-bulk AML blasts, 1-7−CD99 low colonies). FIG. 6F is a graph showing that in CD34+ expressing AMLs (n=54), CD99 expression was higher in the LSC-enriched CD34+CD38− fraction. Error bars represent±SD. * represents p<0.0001 (paired t-test),

FIGS. 7A-7E demonstrate that CD99 promotes disease aggressiveness in vivo but improves patient outcomes in the context of chemotherapy. FIG. 7A is a graph showing that knockdown of CD99 in HL60 cells with two shRNAs (2.3-fold and 8.3-fold with #61 and #59, respectively) did not significantly alter proliferation kinetics in vitro Error bars represent±SD. FIG. 7B is a survival curve showing that there was a significant improvement in overall survival (OS) of sublethally irradiated NSG mice transplanted with MOLM13 cells transduced with shRNA#59 as compared to vector control (58 and 34 days, respectively, p=0.0033, n=5 per group, representative of three independent sets of transplants). FIG. 7C is a graph showing that the surface expression of CD99 correlated with disease burden as measured by % blasts in the peripheral blood of AML patients (n=31, R2=0.2476,p=0.0051, Pearson Correlation). FIG. 7D presents survival curves showing that, in 358 AML patients randomized to standard or intensified dose Daunorubicin (DNR) on the Eastern Cooperative Oncology Group (ECOG) 1900 clinical trial, high CD99 transcript expression correlated with improved OS in the standard dose DNR group (25.0 and 10.4 months for CD99 high and CD99 low, respectively, p=0.000585, left top panel). Intensification of DNR mitigated the poor prognostic import of having low CD99 expression (to 21.3 as compared with 23.3 months for CD99 high, p=0.755, left bottom panel). FIG. 7E presents survival curves showing that, in the DNMT3a or NMI' mutant or HILL-translocated subgroup described to specifically benefit from DNR intensification, DNR intensification improved OS in the CD99 low group (12.3 to 20.2 months, p=0.007, right top panel) but not the CD99 high group (18.1 to 17.5 months, p=0.701, right bottom panel). Log-rank test used for all survival comparisons.

FIGS. 8A-8D demonstrate that CD99 Promotes Transendothelial Migration and Mobilization of Leukemic Blasts, FIG. 8A is a bar graph of HL60 cells that were transduced to overexpress CD99 (7-fold) using a tetracycline inducible lentiviral vector and seeded on human umbilical vein endothelial cells (HUVECs) grown to confluence on transwell inserts (8 pore size). These data show that CD99 overexpression led to a significant increase in transmigration efficiency as measured at four hours and 28 hours. FIG. 8B is a bar graph showing that, after 72 hours, the remaining unmigrated cells had a significantly lower level of CD99 expression as compared with the migrated cells, FIG. 8C is a bar graph showing that primary AML specimens taken from the peripheral blood (n=9) exhibit a significantly higher level of expression of CD99 as compared with bone marrow (n=31). FIG. 80 is a graph of flow cytometry data showing CD99 expression on engrafted cells at 10 weeks following xenografting of human AML sample UPenn 2741 into ten sublethally irradiated NSG mice. These data demonstrate that, in a paired analysis, CD99 expression was significantly higher on engrafted tumor cells circulating in the peripheral blood as compared with those in the bone marrow. Error bars represent±SD. * p<0.05, * p<0.01. ** p<0,001, **** p<0.0001 (paired t-test for panel d, unpaired t-test for all others).

FIGS. 9A-9H demonstrate that anti-CD99 monoclonal antibodies mAbs) are directly cytotoxic to and MDS cells and that such antibody-mediated cytotoxicity is effector- and complement-independent. FIG. 9A (left panel) presents bar graphs of relative cell numbers following incubation of leukemic blasts from AML specimen AU 5.1 with anti-CD99 mAb clone 12E7 for 72 hours at doses of 10 μg/ml or 20 μg/ml. These data show a significant decrease in cell number following incubation with 12E7 antibody as compared to incubation with an IgGll isotype control antibody, FIG. 9A (right panel) presents bar graphs of relative cell numbers following incubation of lineage negative CD34+CD38− cells from a primary MDS patient sample with anti-CD99 mAb clone 12E7 for 72 hours. These data show a significant decrease in cell number following incubation with 12E7 antibody as compared to incubation with an IgGl isotype control antibody. FIG. 9B is a graph of relative cell number following incubation of MOLM13 cells with anti-CD99 mAb H036-1.1 (Abeam. Cambridge, MA) for 72 hours. These data show a dose dependent decrease in cell number with an IC50=186 ng/ml. FIG. 9C is a bar graph of activated caspase 3 (aCaspase3) following incubation of MOLM13 cells with H036-1.1 at 5 μg/mL(micrograms per mL). These data show an increase in aCaspase3, which is indicative of H036-1.1-mediated induction of cell death via apoptosis. FIG. 9D is a graph of relative cell numbers of MOLM13 cells, cord blood (CB) HSCs, and HUVECs following 72 hour incubation with anti-CD99 MAb H036-1.1. These data show that the IC50 was 186 ng/ml for MOLM13 cells and 5201 ng/ml for CB HSCs, which evidences a substantial therapeutic window. The IC50 was not reached with HUVECs with dosing of up to 20,000 ng/mi. The experiment was repeated with MSK MDS-001 CD34+CD38− cells compared to non AML adult bone marrow CD34+CD38− cells and HUVEC cells following exposure to anti-CD99 mAb H036-1.1, as shown in FIG. 12. Similarly, a broad therapeutic window was demonstrated as relative cell numbers were dramatically decreased for MSK MDS-001 CD34+CD38−, while adult BM CD34+CD38− and HUVEC cells were much less affected. FIG. 9E is a graph of human chimerism in peripheral blood (PB) following ex viva treatment of AML specimen UPenn 2741 with H036-1,1 for 45 minutes prior to transplantation into sublethally irradiated NSG mice. These data show a significant decrease in tumor engraftment as measured by human chimerism in PB at eight weeks. FIG. 9F (left panel) presents the results of immunofluorescence analysis (CD99-green, DAN-blue) of HL60 cells following treatment with anti-CD99 mAb (12E7), which revealed marked cell surface capping of CD99 at 105 minutes. FIG. 9F (right panel) is a bar graph of relative cell number following incubation of MOLM13 cells for 72 hours in the presence of a cross-linking anti-IgG secondary mAb, which shows a significant potentiation of the cytotoxicity of anti-CD99 mAb (IgG-isotype) and is consistent with an IgG-mediated enhancement in anti-CD99-mediated aggregation of cell surface CD99 and resulting promotion of MOLM13 cell death.

FIG. 9G is an autoradiograph showing the activation of Src-family kinases (SFKs, pSrc[Y41.6]) following incubation of MOLM13 cells with anti-CD99 MAb H036-1.1. FIG. 911 is a graph of relative number after preincubation of MOLM13 cells for three hours with the SEK anti-CD99 antibody PP2 (20 UM) followed by a 48 hour incubation with H036-1.1, These data show a significant decrease in cytotoxicity (IC50 476 nen:it at 48 hours) in the absence of PP2, IC50 not reached in the presence of PP2; *p<0.05, **p<0.01, ***p<0.001, ****p<0.0001 (unpaired Student's t-test)), which is consistent with the down-regulation of cellsurface CD99 during the preincubation step of that results in decreased H036-1.1-mediated CD99 aggregation and cell death.

FIGS. 10A-10C, FIG. 10A is a bar graph showing the relative cell number for 15 AML cell lines following 48 hr incubation with anti-CD99 antibody 12E7. Of the 15 AML cell lines tested, AML 5Q, NB4, KCL22, MOLM13, HL60, MOLM14, NOMO1, U937, MonoMael, KG la, and AML 14 were found to be susceptible to growth inhibition following CD99 ligation with 12E7 antibody. Four cell lines tested, KBM5, SET2, KU812, and K562 were found to be unresponsive to CD99 ligation with 12E7 antibody. FIG. 10B are flow cytometry plots showing relative expression of c-kit and CD99 in AML cell lines HL60, K562, KBM5, SET2, and KU812 cells (top panel) and bar graphs showing relative cell number after 48 hour incubation with IgGIK or anti-CD99 12E7 antibody (bottom panel). HL60 is a representative “sensitive” cell line, with high CD99 and low c-kit expression. “Resistant” cell lines either do not express CD99 (K562 and KBM5), express high levels of c-kit (SEM2 and KU812), are Bcr-abl positive (K,562/KBM5/K11812), or are JAK2 positive (SEM2). These data demonstrate that low CD99 expression or high c-kit expression are predictive for resistance to cytotoxicity mediated by anti-CD99 antibody 12E7. FIG. 10C is a graph of transendothelial migration kinetics of CD99+ AML cells in the presence of anti-CD99 antibodies 12E7, HEC2,1DN16, and 1⁻1036, indicating that in the absence of secondary antibody, certain anti-CD99 antibodies do not inhibit transendothelial migration of AML cells, which is known to be mediated by CD99.

FIG. 11, upper panel, shows a schematic for combined ex-vivo pretreatment and in vivo treatment with anti-CD99 MAb H036-1.1 of UPenn 2522 xenografted Akfl, mice. FIG. 11, lower panel shows evaluation of human chimerism in bone marrow of AML Blast UPenn 2522 xenografted mice after 5 months. Either pre-coating of AML blasts prior to xenograft or in vivo treatment of xenografted mice with an anti-CD99 MAb eliminated AML xenografts, while 4/5 control animals engrafted leukemia.FIG, 12 is a graph of relative cell numbers of MSK MDS-001 CD34+CD38− cells, adult BM CD34+CD38− cells, and HUVECs following 72 hour incubation with anti-CD99 MAb H036-1.1. The anti-CD99 MAb was significantly more toxic to the MDS-001 CD34+CD38− cells demonstrating a good therapeutic window for treatment of leukemic cells.

### DETAILED DESCRIPTION

The present disclosure is based, in part, upon the results of transcriptome analysis of hematopoietic stem cells (HSCs) from low risk myelodysplastic syndrome (MDS) patients and normal bone marrow (BM) cells from normal adult donors. Out of 3,258 differentially-expressed transcripts identified in that transcriptome analysis, 25 differentially expressed cell surface transcripts were detected. Flow cytometric analysis of 37 de novo MDS cases confirmed dysregulated cell surface protein expression of: CD99 in 73% of patient samples. Flow-cytometric analysis of the same 25 cell surface proteins was also performed in 26 treatment-related MDS patient samples revealing that: CD99 was overexpressed in 85% of patient samples.

Based upon these preliminary observations that the expression of certain cell-surface proteins, in particular the cell-surface marker CD99, is elevated in both hematopoietic stem cells (HSCs) from low risk myelodysplastic syndrome (MDS) patients as well as in treatment-related MDS patient samples, the potential therapeutic efficacy of antibodies having binding specificity for the extracellular domain of CD99 was assessed in MDS cell lines and primary cells as well as in AML cell lines and primary AML blasts. In the inventors' experiments, CD99 was essentially the most frequently overexpressed marker in both MDS and AML and it was overexpressed considerably (average about 7-fold over control).

As described herein, ligation of CD99 on MDS cells with certain anti-CD99 antibodies, such as the anti-CD99 antibody 12E7: (i) resulted in a time-dependent decrease in cell number; (ii) induced cell death in those anti-CD99 antibody ligated MDS cells (as evidenced by an increase in annexin V positivity); (iii) reduced cell surface expression levels of CD99; and (iv) reduced levels of certain myeloid specific differentiation markers, such as CD14 and CM lb. Similarly, ligation of CD99 on primary CD34⁺ MDS cells with certain anti-CD99 antibodies, such as the anti-CD99 antibody 12E7, was cytotoxic to those primary MDS cells as evidenced by a time-dependent decrease in numbers of cells in cultures grown in the presence of anti-CD99 antibodies relative to numbers of cells in cultures grown either in the absence of antibody or in the presence of an isotype control antibody.

It was also observed that ligation of CD99 on AML cells expressing high levels of CD99 with certain anti-CD99 antibodies, such as the anti-CD99 antibody 12E7, was cytotoxic to those AML cells as evidenced by a time-dependent decrease in the numbers of cells in cultures grown in the presence of anti-CD99 antibodies relative to numbers of cells in cultures grown either in the absence of antibody or in the presence of an isotype control antibody. Similarly, ligation of CD99 on CD99-expressing primary AML blasts with certain anti-CD99 antibodies, such as the anti-CD99 antibody 12E7, was cytotoxic to those primary AML blasts, as evidenced by a time-dependent decrease in cell number, but exhibited only a modest time-dependent effect on normal cord blood FISC cell numbers.

Moreover, as disclosed herein, it was further demonstrated that the cytotoxicity owing to anti-CD99 antibody ligation of CD99 on CD99-expressing MDS cell lines, MDS primary cells. AML cell lines, and primary AML blasts occurred in the absence of antibody eftixtor function, including in the absence of complement-dependent cytotoxicity (CDC) andlor antibody-dependent cell-mediated cytotoxicity (ADCC).

Through these studies, it was discovered that a subset of anti-CD99 antibodies tested exhibited cytotoxic properties on CD99+ cell growth, which resulted from anti-CD99-mediated cytotoxicity through the induction of cell death, such as, for example, apoptosis. Those cytotoxic anti-CD99 antibodies shared the property of ligating cell surface CD99 thereby promoting the aggregation on the cell surface of antibody-bound CD99, which led to its clustering or capping within a localized region of the cell surface. Moreover, the cytotoxicity of, and the apoptosis induced by, certain anti-CD99 antibodies were associated directly with each antibody's capacity to promote CD99 aggregation, clustering, and capping. These results indicated that, desirably, anti-CD99 antibodies that are effective in inhibiting CD99+ AML and CD99+ MDS should possess one or more of the properties of: (i) inducing CD99 aggregation; (ii) inducing CD99 clustering; and (iii) inducing CD99 capping on the surface of CD99+ MDS and AML cells.

Based upon these and other discoveries, which are described in further detail herein, the present disclosure provides:
(1) Anti-CD99 antibodies, and compositions comprising one or more anti-CD99 antibodies, for the treatment of acute myeloid leukemias and myelodysplastic syndromes, wherein suitable anti-CD99 antibodies ligate (i.e., bind to) surface-expressed CD99; inhibit growth of CD99+ AML and/or AADS cells; facilitate the aggregation, clustering, and/or capping of ligated CD99; and promote cytotoxicity of AML and/or MDS cells through the direct induction of cell death such as, for example, through apoptosis, necrosis/necroptosis, autophagic cell death, endoplasmic reticulum-stress associated cytotoxicity, or other cell death mechanism such as mitotic catastrophe, paraptosis, pyroptosis, pyronecrosis, and entosifs;
(2) Methods for generating and for identifying anti-CD99 antibodies that are suitable for the treatment of an acute myeloid leukemia and/or a myelodysplastic syndrome;
(3) Methods for identifying a patient having acute myeloid leukemia and/or the myelodysplastic syndrome that is susceptible to treatment with an anti-CD99 antibody and/or a compositions comprising one or more anti-CD99 antibody(ies);
(4) Methods for inducing apoptosis in a CD99′ cell that is associated with an acute myeloid leukemia and/or a myelodysplastic syndrome, such as a CD99 leukemic stem cell and/or a hematopoietic stem cell; and
(5) Methods for the treatment of a CD99⁺ acute myeloid leukemia and/or a CD99 myelodysplastic syndrome which exhibit elevated levels of CD99,

As described in greater detail herein, these antibodies, compositions, and methods for treating acute myeloid leukemia and/or a myelodysplastic syndrome derive from the newly-discovered and presently-disclosed relationships between: (1) elevated expression of CD99 in tissues and/or cells that are associated with an acute myeloid leukemia (including primary AML cells, blasts, and leukemic stem cells (LSC)) and/or are associated with a de novo or treatment-related myelodysplastic syndrome (including primary MDS cells, blasts, and/or hematopoietic stem cells (HSC)); (2) the sensitivity of such CD99+ AML and MDS tissues and cells to ligation by certain anti-CD99 antibodies and the consequent aggregation, clustering, and capping of cellular CD99 when ligated to such anti-CD99 antibodies; and (3) the cytotoxicity resulting from anti-CD99 ligation, and consequent aggregation, clustering, and capping, which is mediated by anti-CD99 binding to cell surface-expressed CD99 and the cell death (e.g., apoptosis) that is induced as a consequence of that CD99 aggregation.

Anti-CD99 Antibodies/or Ligation-Mediated Clustering of CD99 CD99⁺ AML and MDS Cells

The present disclosure provides cytotoxic anti-CD99 antibodies that may be suitably employed in the presently disclosed methods for inhibiting the proliferation of or inducing apoptosis CD99+ AML and/or MDS cells, which are suitable for the treatment of patients afflicted with an ANIL and/or an MDS composed of cells that express elevated levels of CD99.

The cytotoxic anti-CD99 antibodies disclosed herein exhibit the following properties:
1. Binding affinity for the extracellular domain of CD99 when expressed on the surface of a CD99⁺ AML and/or MDS cell;
2. Promoting on or more of the aggregation, clustering, and capping of antibody bound CD99 on the surface of a. CD99⁺ AML andlor MDS cell; and
3. Inducingcelt death in a CD99⁺ AML and/or MDS cell when bound to CD99 on the surface of a CD99⁺ AML and/or MDS cell.

It will be understood that the promotion of aggregation, clustering, and/or capping by an antibody bound to CD99 on the surface of a CD99⁺ AML andlor MDS cell is predictive of the cytoti.)xicity of that antibody and its capacity to induce cell death, whether by apoptosis, necrosis/necroptosis, autophagic cell death, endoplasmic reticulum-stress associated cytotoxicity, or other cell death mechanism such as mitotic catastrophe, paraptosis, pyroptosis, pyronecrosis, and entosifs. See, Kroemer et al., Cell Death Differ. 16(1):3-11 (2009). In any event, cell death can be observed directly with or without the intermediary assessment of aggregation, clustering, and/or capping.

Exemplary antibodies that may be suitably employed in these methods include the murine IgG₁ antibodies 12E7 (Levy et al., Proc. Alatt. Acad. Sci. 76:6552 (1979)) and O03 (Dracopoli et al., Am. J. Hum. Genet. 37:199 (1985)), which both bind to the extracellular domain of CD99 at the epitope “DGEN” (SEQ ID NO: 7), which is defined by amino acids 61-64 of SEQ ID NO' 77′, amino acids 45-48 of SEQ ID NO: 4, and amino acids 61-64 of SEQ ID NO: 6 and the murine IgM H036-1.1 antibody, which binds to the extracellular domain of CD99 at the epitope DL)PRPPNPPK (SEQ ID NO: 12).

In contrast, the murine IgG₁ antibody DN16 (Hahn et al., J. Immunol. 159:2250 (1997)), which binds to the extracellular domain of CD99 at the epitope “LPDNENKK” (SEQ ID NO: 8) that is defined by amino acids 32-39 of SEQ ID NOs: 2 and 6, but is not present in the amino acid sequence of SEQ IL) NO: 4 (wherein amino acids 32-39 contain the amino acid sequence “LPGDDFDL”(SEQ ID NO: 9)) is unsuitable for the presently disclosed methods because, while binding to the extracellular domain of CD99, exhibits a limited capacity to promote the aggregation of antibody bound CD99 and a correspondingly low level of cytotoxicity, which antibody-mediated aggregation, cytotoxicity, and apoptosis can be augmented by binding the anti-CD99 antibody-associated CD99 with a secondary anti-IgG antibody, which, because of its bivalency, brings two anti-CD99 antibody bound CD99 molecules into close proximity.

A peptide scanning experiment of overlapping 15-mer peptides of a CD99 extracellular domain was employed to further identify potential epitopes required for anti-CD99 mA.b cytotoxicity, Results are shown in Table 1. MAbs 12E7 and F8 both recognized overlapping peptides comprising the epitope D.A.VVDGEND (SEQ ID NO: 10). MAb O13 recognized overlapping peptides comprising the epitope AVVDGEN (SEQ ID NO: 11). MAb H036-1.1 recognized overlapping peptides comprising the epitope DDPRPPNPFK (SEQ lID NO: 12). MAb 3B2 recognized overlapping peptides comprising the epitope LPD (SEQ ID NO: 13). MAb recognized overlapping peptides comprising the epitope DALPDN (SEQ ID NO: 14). The sequence of the extracellular domain of which the 15-mer peptides are fragments is SEQ If) NO: 15.

Similarly, the tnurine IgG antibodies 3B2 and F8 are unsuitable for the presently disclosed methods because, while binding to the extracellular domain of CD99, do not promote the aggregation of antibody bound CD99 and are not cytotoxic to antibody-bound AML and/or MDS cells as a result of their inability to mediated aggregation and induce cell death. Moreover, binding of a secondary anti-IgG antibody to 3B2- and F8-antibody bound CD99 is ineffective in promoting anti-CD99 antibody-mediated aggregation or cytotoxicity.

Presented in Table I are the properties of each of the 12E7, O13, and H036-1.1 anti-CD99 antibodies, which exhibit the desired properties of (1) binding to the extracellular domain of CD99 when expressed on the surface of an AML and/or MDS cell; (2) promoting at least one of the aggregation, clustering, and capping of surface-expressed CD99 on AML and/or MDS cells. These antibodies are cytotoxic to CD99⁺ AML and/or MDS cells and have been demonstrated to induce cell death in CD99 AML and/or MDS cells.

Also presented in Table 1 are properties of the DN16, 3B2, and F8 anti-CD99 antibodies, which exhibit the desired property of binding to the extracellular domain of CD99 when expressed on the surface of an AML and/or MDS cell but do not promote the aggregation, clustering, and capping of surface-expressed CD99 on AML and/or MDS cells. These antibodies are not cytotoxic to CD99⁺ AML and/or MDS cells and lack the capacity to induce cell death in CD99 ⁺ AML and/or MDS cells.

Thus, it will be understood that cytotoxic antibodies sharing with the 12E7, O13, and H036-1.1 anti-CD99 antibodies the capacity to promote aggregation/clustering/capping and, consequently, to induce cell death in CD99 AML and/or MDS cells are within the scope of the present disclosure and can be suitably employed in the presently disclosed methods for inducing /cytotoxicity in a CD99+ AML and/or MDS cell or for treating an AML and/or MDS patient exhibiting AML and or MDS-associated cells that express elevated levels of CD99.

In contrast, it will also be understood that non-cytotoxic antibodies sharing with the DN16, 3B2, and F8 anti-CD99 antibodies the limited capacity or inability to promote aggregation/clustering/capping or to induce cell death in CD99⁻ AML and/or MDS cells are outside of the scope of the present disclosure and the present methods.

TABLE 1
Properties of Anti-CD99 Antibodies

| | | | | | Secondary |
|---|---|---|---|---|---|
| | | | | Induces | anti-mouse |
| | | | | Cytotoxicity | IgG Cross- |
| | | | Promotes | via Inducing | linking |
| | | | CD99 | Apoptosis in | Antibody |
| Antibody | Sequence of | | Aggregation, | CD99+ AML | Induces or |
| Designation | CD99 Binding | Mediates CD99 | Clustering, | and/or MDS | Augments |
| Antibody Isotype | Epitope | Ligation | and Capping | Cells | Cytotoxicity |
| 12E7 | DAVVDGEND | Yes | Yes | Yes | Yes |
| Murine IgG1 | | | | | |
| O13 | AVVDGEN | Yes | Yes | Yes | Yes |
| Murine IgG1 | | | | | |
| H036-1.1 | DDPRPPNPPK | Yes | Yes | Yes | n/a (IgM |
| Murine IgM | | | | | isotype) |
| DN16 | DALPDN | Yes | No | No | Yes |
| murine IgG | | | | | |
| 3B2 | LPD | Yes | No | No | No |
| Murine IgG | | | | | |
| F8 | DAVVDGEND | Yes | No | No | Not tested |
| Murine IgG | | | | | |

Anti-CD99 antibodies that may be suitable to be employed in the methods disclosed herein bind to the extracellular domain of human CD99 with an IC50 of from about 100 lig/nil to about 10 μg/ml or from about 250 ng/ml to about 5 μg/ml or from about 500 ng/ml to about 1 μg/ml.

Anti-CD99 antibodies that are suitable for practicing the methods of the present disclosure are preferably monoclonal antibodies and may be human, humanized or chimeric monoclonal antibodies, comprising single chain antibodies, Fab fragments, F(abr) fragments, fragments produced by a Fab expression library, and/or binding fragments of any of the 12E7, O13, and 1-1036-1.1 anti-CD99 antibodies described herein or of other antibodies, whether previously described or newly developed, so long as those anti-CD99 antibodies share with the 12E7, O13, and 1-1036-1.1 anti-CD99 antibodies: (1) a binding affinity for the extracellular domain of CD99 when expressed on the surface of a CD99^{÷} AML and/or MDS cell; (2) the promotion of aggregation, clustering, andlor capping of antibody bound CD99 on the surface of a CD99⁺ AML and/or MDS cell; and (3) the inducing of cell death in a CD99^{÷} AML and/or MDS cell when bound to CD99 on the surface of a CD99⁺ AML andlor MDS cell. Because of the close correlation between one or more of aggregation, clustering, and capping and eytotoxicity, aggregation, clustering, and capping can serve as markers for cytotoxicity and, more specifically, for cell death. Thus, antibodies previously identified to cause one or more of aggregation, clustering, and capping can be used to promote cytotoxicity by inducing cell death.

Anti-CD99 antibodies of the present disclosure can be created by traditional means or may be generated by recombinant techniques. Monoclonal antibodies are obtained from a population of substantially homogeneous antibodies, i.e. the individual antibodies comprising the population are identical except for possible naturally occurring mutations that may be present in minor amounts. Thus, the modifier “monoclonal” indicates the character of the antibody as not being a mixture of discrete antibodies.

The anti-CD99 monoclonal antibodies of the present disclosure can be made using the hybridoma method first described by Kohler et al., Nature 256:495 (1975), or may be made by recombinant DNA methods (U.S. Pat. No. 4,816,567).

In the hybridoma me hod, a mouse or other appropriate host animal, such as a hamster, is immunized to elicit lymphocytes that produce or are capable of producing antibodies that will specifically bind to the protein used for immunization. Antibodies to CD99 may be raised in animals by multiple subcutaneous (sc) or intraperitoneal (ip) injections of CD99 and an adjuvant, CD99 may be prepared using methods well-known in the art, some of which are further described herein, For example, recombinant production of human and mouse CD99 is described below. In one embodiment, animals are immunized with a CD99 fused to the Fe portion of an immunoglobulin heavy chain, In another ernbodiment, animals are immunized with a CD99-IgGI fusion protein. Animals ordinarily are immunized against immunogenic conjugates or derivatives of CD99 with monophosphoryl lipid A (MPL)/trehalose dicrynomyeolate (TDM) (Rihi Immunochem. Research, Inc., Hamilton, Mont.) and the solution is injected intradermally at multiple sites, Two weeks later the animals are boosted. 7 to 14 days later animals are bled and the serum is assayed for anti-CD99 titer. Animals are boosted until titer plateaus.

Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp, 59-103 (Academic Press, 1986)).

Hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient

Myeloma cells may be those that fuse efficiently, support stable high-level production of antibody by the selected antibody-producing cells, and are sensitive to a medium such as HAT medium. Myeloma cell lines may be murine myeloma such as those derived from MOPC-21 and 111PC-11 mouse tumors available from the Salk Institute Cell Distribution Center, San Diego, Calif. IJS,A., and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol. 133:3001 (1984); Brodeur et at, Monoclonal Antibody Production Techniques and Applications, pp. 51-63 (Marcel Dekker, Inc., New York, 1987)).

Culture medium in which hybridoma cells are growing is assayed for production of monoclonal antibodies directed against CD99. The binding specificity of monoclonal antibodies produced by hybridoma cells may be determined by immunoprecipitation or by an in vitro binding assay, such as radioimmunoassay (RIA) or enzyme-linked immunoadsorbent assay (ELISA).

The binding affinity of the monoclonal antibody can, for example, be determined by the Scatchard analysis of Munson et al., Anal. Biochem. 107:220 (1980). After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

The anti-CD99 antibodies of the present disclosure can be made by using combinatorial libraries to screen for synthetic antibody clones with the desired activity or activities. In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can he further enriched by additional cycles of antigen adsorption/elution. Any of the anti-CD99 antibodies of the present disclosure can be obtained by designing a suitable antigen screening procedure to select for the phage clone of interest followed by construction of a full length anti-CD99 antibody clone using the Fv sequences from the phage clone of interest and suitable constant region (Fe) sequences described in Kabat et al., Sequences of Proteins of Immunological Interest, Fifth Edition, NIH Publication 91-3242, Bethesda. Md. (1991), vols. 1-3.

The antigen-binding domain of an antibody is formed from two variable (V) regions of about 110 amino acids, one each from the light (VL) and heavy (VH) chains, that both present three hypervariable loops or complementarity-determining regions (CDRs). Variable domains can be displayed functionally on phage, either as single-chain Fv (scFv) fragments, in which VH and VL are covalently linked through a short, flexible peptide, or as Fab fragments. in which they are each fused to a constant domain and interact non-covalently, as described in Winter et al., Ann. Rev. Immunol. 12:433-455 (1994). As used herein, scFv encoding phage clones and Fab encoding phage clones are collectively referred to as “Fv phage clones” or “Fv clones”.

Repertoires of VH and VL genes can be separately cloned by polymerase chain reaction (PCR) and recombined randomly in phage libraries, which can then be searched for antigen-binding clones as described in Winter et al., Ann. Rev. Inununol. 12: 433-455 (1994). Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EBO J. 12:725-734 (1993). Finally, naive libraries can also he made synthetically by cloning the unrearranged V-gene segments from stem cells, and using PCR primers containing random sequence to encode the highly variable CDR3 regions and to accomplish rearrangement in vitro as described by Hoogenboora and Winter, J. Mol. Biol. 227:381-388 (1992).

Filamentous phage is used to display antibody fragments by fusion to the minor coat protein p111. The antibody fragments can be displayed as single chain Fv fragments, in which VH and VL domains are connected on the same polypeptide chain by a flexible polypeptide spacer, e.g., as described by Marks et al., J. Mol. Biol. 222:581-597 (1991), or as Fab fragments, in which one chain is fused to pill and the other is secreted into the bacterial host cell periplasm where assembly of a Fa :b-coat protein structure which becomes displayed on the phage surface by displacing some of the wild type coat proteins, e.g., as described in Hoogenboom et al., Nucl. Acids Res. 19:4133-4137 (1991).

in general, nucleic acids encoding antibody gene fragments are obtained from immune cells harvested from humans or animals. If a library biased in favor of anti-CD99 clones is desired, the individual is immunized with CD99 to generate an antibody response, and spleen cells and/or circulating B cells other peripheral blood lymphocytes (PBLs) are recovered for library construction. A human antibody gene fragment library may be biased in favor of anti-CD99 clones is obtained by generating an anti-CD99 antibody response in transgenic mice carrying a functional human immunoglobulin gene array (and lacking a functional endogenous antibody production system) such that CD99 immunization gives rise to B cells producing human antibodies against CD99. The generation of human antibody-producing transgenic mice is described below.

Additional enrichment for anti-CD99 reactive cell populations can be obtained by using a suitable screening procedure to isolate B cells expressing CD99-specific membrane bound antibody, e.g., by cell separation with CD99 affinity chromatography or adsorption of cells to fluorochrome-labeled CD99 followed by flow-activated cell sorting (FACS),

Alternatively, the use of spleen cells and/or B cells or other PBLs from an unimmunized donor provides a better representation of the possible antibody repertoire, and also permits the construction of an antibody library using any animal (human or non-human) species in which CD99 is not antigenic. For libraries incorporating in vitro antibody gene construction, stem cells are harvested from the individual to provide nucleic acids encoding unrearranged antibody gene segments. The immune cells of interest can be obtained from a variety of animal species, such as human, mouse, rat, lagotnorpha, luprine, canine, feline, porcine, bovine, equine, and avian species, etc.

Nucleic acid encoding antibody variable gene segments (including VH and VL segments) are recovered from the cells of interest and amplified. In the case of rearranged VH and VL gene libraries, the desired DNA can be obtained by isolating genomic DNA or mRNA from lymphocytes followed by polymerase chain reaction (POO with primers matching the 5′ and 3′ ends of rearranged VH and VL genes as described in Orlandi et al., Proc. Natl. Acad. Sci. U.S.A. 86:3833-3837 (1989), thereby making diverse V gene repertoires for expression. The V genes can be amplified from cDNA and genomic DNA, with back primers at the 5′ end of the exon encoding the mature V-domain and forward primers based within the J-segment as described in Orlandi et al. (1989) and in Ward et al., Nature 341:544-546 (1989). However, for amplifying from cDNA, back primers can also be based in the leader exon as described in Jones et al., Biotechnol. 9:88-89 (1991), and forward primers within the constant region as described in Sastry et al., Proc. Nall. Acad. Sci. USA. 6:5728-5732 (1989). To maximize complementarity, degeneracy can be incorporated in the primers as described in Orlandi et al. (1989) or Sastry el al, (1989). Library diversity may be maximized by using PCR primers targeted to each V-gene family in order to amplify all available VII and VL arrangements present in the immune cell nucleic acid sample, e.g, as described in the method of Marks et al., J. Mol. Biol. 222:581-597 (1991) or as described in the method of Oruro et al., Nucleic Acids Res. 21:4491-4498 (1994 For cloning of the amplified DNA into expression vectors, rare restriction sites can be introduced within the PCR primer as a tag at one end as described in Orlandi et al. (1989), or by further PCR amplification with a tagged primer as described in Clackson et al., Nature 352:624-628 (1991).

Repertoires of synthetically rearranged V genes may be derived in vitro from V gene segments. Most of the human VH-gene segments have been cloned and sequenced (reported in Tomlinson et al., J Mol. Biol. 227:776-798 (1992)), and mapped (reported in Matsuda et al., Nature Genet. 3:88-94 (1993); these cloned segments (including all the major conformations of the H1 and 112 loop) can be used to generate diverse VH gene repertoires with PCR primers encoding H3 loops of diverse sequence and length as described in Hoogenboom and Winter, J. Mol. Biol. 227:381-388 (1992). VH repertoires can also be made with all the sequence diversity focused in a long VH loop of a single length as described in Barbas et al., Proc. Natl. Acad. Sci. U.S.A. 89:4457-4461 (1992). Human Vκ and Vλ segments have been cloned and sequenced (reported in Williams and Winter, Eur. J. immunol. 23:1456-1461 (1993)) and can be used to make synthetic light chain repertoires. Synthetic V gene repertoires, based on a range of VH and VL folds, and L3 and H3 lengths, will encode antibodies of considerable structural diversity. Following amplification of V-gene encoding DNAs, germline V-gene segments can be rearranged in vitro according to the methods of Hoogenboom and Winter, J. Mol. Biol. 227:381-388 (1992).

Repertoires of antibody fragments can be constructed by combining VH and VL gene repertoires together in several ways. Each repertoire can be created in different vectors, and the vectors recombined in vitro, e.g., as described in Hogrefe et al., Gene 128:119-126 (1993), or in vivo by combinatorial infection, e.g., the loxP system described in Waterhouse et al., Nucl. Acids Res. 21:2265-2266 (1993). The in vivo recombination approach exploits the two-chain nature of Fab fragments to overcome the limit on library size imposed by E. coli transformation efficiency. Naive VH and VL repertoires are cloned separately, one into a phagemid and the other into a phage vector. The two libraries are then combined by phage infection of phagemid-containing bacteria so that each cell contains a different combination and the library size is limited only by the number of cells present (about 10¹² clones). Both vectors contain in vivo recombination signals so that the VH and VL genes are recombined onto a single replicon and are co-packaged into phage virions, These huge libraries provide large numbers of diverse antibodies of good affinity (K_{d}⁻¹ of about 10⁸ M).

Alternatively, the repertoires may be cloned sequentially into the same vector, e,g., as described in Barbas et al., Proc. Nati, Acad. Sci. U.S.A. 88:7978-7982 (1991), or assembled together by PCR and then cloned, e.g. as described in Clackson et al., Nature 352:624-628 (1991). PCR assembly can also be used to join VH and VL DNAs with DNA encoding a flexible peptide spacer to form single chain Fv (scFv) repertoires. In yet another technique, “in cell PCR assembly” is used to combine VII and VL genes within lymphocytes by PCR and then done repertoires of linked genes as described in Embleton et al., Nucl. Acids Res. 20:3831-3837 (1992).

The antibodies produced by naive libraries (either natural or synthetic) can be of moderate affinity (K_{d}⁻¹ of about 10⁶ to 10⁷ M⁻¹), but affinity maturation can also be mimicked in vitro by constructing and reselecting from secondary libraries as described in Winter et al, (1994), supra. For example, mutations can be introduced at random in vitro by using error-prone polymerase (reported in Leung et. al., Technique 1:11-15 (1989)) in the method of Hawkins et al., J. Mot. Biol. 226:889-896 (1992) or in the method of Gram et al., Proc. Natl. Acad. Sci. U.S.A. 89:3576-3580 (1992), Additionally, affinity maturation can be performed by randomly mutating one or more CDRs, e.g., using PCR with primers carrying random sequence spanning the CDR of interest, in selected individual Fv clones and screening for higher affinity clones. PCT Patent Publication No. WO 1996/07754 (published Mar. 14, 1996) described a method for inducing mutagenesis in a complementarity determining region of an immunoglobulin light chain to create a library of light chain genes. Another effective approach is to recombine the VII or VI.: domains selected by phage display with repertoires of naturally occurring V domain variants obtained from unimmunized donors and screen for higher affinity in several rounds of chain reshuffling as described in Marks et al., Biotechnol. 10:779-783 (1992), This technique allows the production of antibodies and antibody fragments with affinities in the 10⁻⁹ M range. CD99 nucleic acid and amino acid sequences are known in the art and are presented herein in Table 2A, Nucleic acid sequence encoding the CD99 can be designed using the amino acid sequence of the desired region of CD99.

Nucleic acids encoding CD99 can be prepared by a variety of methods known in the art. These methods include, but are not limited to, chemical synthesis by any of the methods described in Engels et al., Agnew. Chem, Int. Ed. Engl. 28:716-734 (1989), such as the triester, phosphite, phosphoramidite and H-phosphonate methods. n one embodiment, codons preferred by the expression host cell are used in the design of the CD99 encoding DNA. Alternatively, DNA encoding the CD99 can be isolated from a genomic or cDN.A library.

Following construction of the DNA molecule encoding the CD99, the DNA molecule is operably linked to an expression control sequence in an expression vector, such as a plasmid, wherein the control sequence is recognized by a host cell transformed with the vector. In general, plasmid vectors contain replication and control sequences which are derived from species compatible with the host cell. The vector ordinarily carries a replication site, as well as sequences which encode proteins that are capable of providing phenotypic selection in transformed cells. S⁻uitable vectors for expression in prokaryotic and eukaryotic host cells are known in the art and some are further described herein. Eukaryotic organisms, such as yeasts, or cells derived from multicellular organisms, such as mammals, may be used.

Optionally, the DNA encoding the CD99 is operably linked to a secretory leader sequence resulting in secretion of the expression product by the host cell into the culture medium. Examples of secretory leader sequences include stil, ecotin, lamB, herpes GD, lpp, alkaline phosphatase, invertase, and alpha factor. Also suitable for use herein is the 36 amino acid leader sequence of protein A (Abrahmsen et al., EMBO J. 4: 3901 (1985)).

Host cells are transfected and preferably transformed with the above-described expression or cloning vectors of this present disclosure and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ precipitation and electroporation. Successful transfection is generally recognized when any indication of the operation of this vector occurs within the host cell. Methods for transfection are well known in the art, and some are further described herein.

Transformation means introducing DNA into an organism so that the DNA is replicable, either as an extrachromosomal element or by chromosomal integrant. Depending on the host cell used, transformation is done using standard techniques appropriate to such cells. Methods for transformation are well known in the art, and some are further described herein.

Prokaryotic host cells used to produce the CD99 can be cultured as described generally in Sambrook et al., supra. Mammalian host cells used to produce the CD99 can be cultured in a variety of media, which is well known in the art and some of which is described herein. The host cells referred to in this disclosure encompass cells in in vitro culture as well as cells that are within a host animal.

Purification of CD99 may be accomplished using art-recognized methods, some of which are described herein. The purified CD99 can be attached to a suitable matrix such as agarose beads, acrylamide beads, glass beads, cellulose, various acrylic copolymers, hydroxymethacrylate gels, polyacrylic and polymethacrylic copolymers, nylon, neutral and ionic carriers, and the like, for use in the affinity chromatographic separation of phage display clones. Attachment of the CD99 protein to the matrix can be accomplished by the methods described in Methods in Enzymology, vol. 44 (1976). A commonly employed technique for attaching protein ligands to polysaccharide matrices, e.g. agarose, dextran or cellulose, involves activation of the carrier with cyanogen halides and subsequent coupling of the peptide ligan.d′s primary aliphatic or aromatic amines to the activated matrix,

Alternatively, CD99 can be used to coat the wells of adsorption plates, expressed on host cells affixed to adsorption plates or used in cell sorting, or conjugated to biotin for capture with streptavidin-coated beads, or used in any other art-known method for panning phage display libraries.

The phage library samples are contacted with immobilized CD99 under conditions suitable for binding of at least a portion of the phage particles with the adsorbent. Normally, the conditions, including pH, ionic strength, temperature and the like are selected to mimic physiological conditions. The phages hound to the solid phase are washed and then eluted by acid, e.g. as described in Barbas et al. Proc. Nall. Acad. Sci. U.S.A. 88:7978-7982 (1991), or by alkali, e.g. as described in Marks et al., J. Mol. Biol. 222:581-597 (1991), or by CD99 antigen competition, e.g. in a procedure similar to the antigen competition method of Ciackson et al., Nature 352:624-628 (1991). Phages can be enriched 20-1,000-fold in a single round of selection. Moreover, the enriched phages can be grown in bacterial culture and subjected to further rounds of selection.

The efficiency of selection depends on many factors, including the kinetics of dissociation during washing, and whether multiple antibody fragments on a single phage can simultaneously engage with antigen. Antibodies with fast dissociation kinetics (and weak binding affinities) can be retained by use of short washes, multivalent phage display and high coating density of antigen in solid phase. The high density not only stabilizes the phage through multivalent interactions, but favors rebinding of phage that has dissociated. The selection of antibodies with slow dissociation kinetics (and good binding affinities) can be promoted by use of long washes and monovalent phage display as described in Bass et al., Proteins 8:309-314 (1990) and in WO 92/09690, and a low coating density of antigen as described in Marks et al., Biotechnol. 10:779-783 (1992).

It is possible to select between phage antibodies of different affinities, even with affinities that differ slightly, for CD99. However, random mutation of a selected antibody (e.g. as performed in some of the affinity maturation techniques described above) is likely to give rise to many mutants, most binding to antigen, and a. few with higher affinity. With limiting CD99, rare high affinity phage could be competed out. To retain all the higher affinity mutants, phages can be incubated with excess biotinylated CD99, but with the biotinylated CD99 at a concentration of lower molarity than the target molar affinity constant for CD99. The high affinity-binding phages can then be captured by streptavidin-coated paramagnetic beads. Such “equilibrium capture” allows the antibodies to be selected according to their affinities of binding, with sensitivity that permits isolation of mutant clones with as little as two-fold higher affinity from a great excess of phages with lower affinity. Conditions used in washing phages bound to a solid phase can also be manipulated to discriminate on the basis of dissociation kinetics.

Fv clones corresponding to CD99 antibodies can be selected by (1) isolating CD99 clones from a phage library as described above, and optionally amplifying the isolated population of phage clones by growing up the population in a suitable bacterial host; (2) selecting CD99 and a second protein against which blocking and non-blocking activity, respectively, is desired; (3) adsorbing the anti-CD99 phage clones to immobilized CD99; (4) using an excess of the second protein to elute any undesired clones that recognize CD99-binding determinants which overlap or are shared with the binding determinants of the second protein; and (5) eluting the clones which remain adsorbed following step (4). Optionally, clones with the desired blocking/non-blocking properties can be further enriched by repeating the selection procedures described herein one or more times.

DNA encoding the hybridoma-derived monoclonal antibodies or phage display Fv clones of the present disclosure is readily isolated and sequenced using conventional procedures (e.g., by using oligonucleotide primers designed to specifically amplify the heavy and light chain coding regions of interest from hybridoma or phage DNA template). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the desired monoclonal antibodies in the recombinant host cells. Review articles on recombinant expression in bacteria of antibody-encoding DNA include Skerra et al., Curr. Opinion in Inununol, 5:256 (1993) and Pluckthun, Immunol. Revs. 130:151 (1992).

DNA encoding the Fv clones of the present disclosure can be combined with known DNA sequences encoding heavy chain and/or light chain constant regions (e.g., the appropriate DNA sequences can be obtained from Kabat et al., supra) to form clones encoding full or partial length heavy and/or light chains. It will be appreciated that constant regions of any isotype can be used for this purpose, including IgG, IgM, IgA, IgD, and. IgE constant regions, and that such constant regions can be obtained from any human or animal species. A Fv clone derived from the variable domain DNA of one animal (such as human) species and then fused to constant region DNA of another animal species to form coding sequence(s) for “hybrid,” frill length heavy chain and/or light chain is included in the definition of “chimeric” and “hybrid” antibody as used herein. In a preferred embodiment, a Fv done derived from human variable DNA is fused to human constant region DNA to form coding s,quence(s) for all human, full or partial length heavy and/or light chains.

DNA encoding anti-CD99 antibody derived from a hybridoma of the present disclosure can also he modified, for example, by substituting the coding sequence for human heavy- and light-chain constant domains in place of homologous murine sequences derived from the hybridoma done as in the method of Morrison et al., Proc. Natl. Acad. Sci. USA, 81:6851-6855 (1984)). DNA encoding a hybridoma or Fv done-derived antibody or fragment can be further modified by covalently joining to the immunoglobulin coding sequence all or part of the coding sequence for a non-immunoglobulin polypeptide. In this manner, “chimeric” or “hybrid” antibodies are prepared that have the binding specificity of the Fv clone or hybridoma clone-derived antibodies of the present disclosure.

The present disclosure further contemplates chimeric derivatives of anti-CD99 antibodies wherein the antibody contains a non-human animal variable region and a human constant region. Chimeric antibody molecules can include, for example, the antigen binding domain from an antibody of a mouse, rat, camelid, or other species, with human constant regions. A variety of approaches for making chimeric antibodies have been described and can be used to make chimeric antibodies containing the immunoglobulin variable region which recognizes the extracellular domain of CD99 on the surface of Aryfli, and/or MDS cells. See, for example, Morrison et al., Proc. Natl. Acad. Sei. U.S.A. 81:6851 (1985); Takeda et al., Nature 314:452 (1985); Cabilly et al., U.S. Pat. No. 4,816,567; Boss et al., U.S. Pat. No. 4,816,397; Tanaguchi et al, European Patent Publication EP171496, European Patent Publication 10 0173494, United Kingdom Patent GB 2177096B, and PCT Patent Publication No. WO 2013/064700, each of which is incorporated herein by reference.

in certain aspects, anti-CD99 antibodies within the scope of the present disclosure include human antigen-binding antibody fragments such as Fab, Fab′, and F(ab′)2′, Fd, single-chain Fvs (scf v). single-chain antibodies, disulfide-linked Fvs (sdFv) and fragments comprising either a V_{I}, or V_{H} domain. CD99-binding antibody fragments, including single-chain antibodies, may include the variable region.(s) alone or in combination with the entirety or a portion of the following: hinge region, C_{H1}, C_{H2}, C_{H3}, and C₁, domains. Also included are CD99-binding fragments containing any combination of va:riabk region(s) with a hinge region, C_{H1}, C_{H2}, and C_{L} domain. The antibodies may be human, mouse, rat, donkey, sheep, rabbit, goat, guinea pig, camelid, horse, or chicken. As used herein, “human” antibodies include antibodies having the amino acid sequence of a human immunoglobulin and include antibodies isolated from human immunoglobulin libraries, from human B cells, or from animals that are transgenic expressing one or more human immunoglob⁻ulins. The generation of camelid antibodies is described, for example, in PCT Patent Publication No. WO 2013/064700.

As disclosed herein, anti-CD99 antibodies that may be suitably employed in the present methods are those for which at least one of the properties of promoting the aggregation, clustering, and/or capping of antibody-bound CD99 on the surface of an AML and/or an MDS cell can be confirmed. It will be appreciated, therefore, that such properties as aggregation, clustering, and/or capping of antibody-bound CD99 may be enhanced by employing bi- or multi-specific anti-CD99 antibodies.

As used herein, each of the terms “bispecific antibody” and “bifunctional antibody” refers to antibodies that recognize two different antigenic epitopes on a CD99 extracellular domain by virtue of possessing at least one first antigen combining site specific for a first epitope, antigen, or hapten, and at least one second antigen combining site specific for a second epitope, antigen, or hapten.

Such antibodies can be produced by recombinant DNA methods or include, but are not limited to, antibodies produced chemically by methods known in the art. Bispecific antibodies include all antibodies or conjugates of antibodies, or polymeric forms of antibodies that are capable of recognizing two different CD99 epitopes. Bispecific antibodies include antibodies that have been reduced and reformed so as to retain their bivalent characteristics and to antibodies that have been chemically coupled so that they can have multiple antigen recognition sites for CD99.

By way of nonlimiting example, bispccific antibodies for use in the present methods can bind to at least one of the CD99 epitopes described herein and may bind to two or more such CD99 epitopes, In some embodiments, one specificity of the antibody has a low affinity, e.g. less than about. 10⁻⁹ binding constant, usually less than about 10⁻⁸ binding constant, and may be more than about 10⁻⁷ binding constant.

Antibodies suitable for practicing the methods of the present disclosure may be bispecific, trispecific, or of greater multispecificity. Further, antibodies of the present disclosure may have low risk of toxicity against granulocyte (neutrophil), NK cells, and CD4+ cells as bystander cells.

Methods for making bispecific antibodies are known in the art. Traditional production of full-length bispecific antibodies is based on the co-expression of two immunoglobulin heavy chain-light chain pairs, where the two chains have different specificities. Mil'stein et al., Nature 305:537-539 (1983). The chains may be connected by a linker, such as those discussed below. A suitable and commonly used linker is (GGGGS)₃.

Because of the random assortment of immunoglobulin heavy and light chains, these hybridomas (quadromas) produce a mixture of different antibody molecules. Purification of an antibody having the desired combination of anti-CD99 epitope binding specificities can be done by affinity chromatography steps. Related isolation procedures are disclosed in PCI Publication No. WO 1993/08829 and in Traunecker et al., EMBO J. 10:3655-3659 (1991).

Bispecific antibodies can be generated by combining two variable regions, a first variable region being specific for a first CD99 epitope and a second variable region being specific for a second CD99 epitope such that the aggregation of ANL and/or MDS cell surface expressed CD99 is enhanced. Such bispecific antibodies can resemble the single-chain bispecific I-cell engager (BiTEO) antibodies described in Bassan, Blood 120(26):5094-5095 (2012) and exemplified by the first in class CD19×CD3 BiTE antibody blinatumomab, which is described in Topp et al., Blood 120(26): 5185-5187 (2012) and Loffler et al., Blood 95(6):2098-2103 (2000).

BiTE® antibodies consist of two different single-chain fv fragments having the structure V_{L1}-V_{H1}-V_{H2}-V_{L2} on a single peptide chain and joined by a glycine-serine linker. BiTE antibody constructs are generally on the order of 60-kDa in size and can be expressed in mammalian cells, such as Chinese hamster ovary (CHO) cells, secreted at high yield in fully active form without requiring renaturation, and purified by a C-terminal histioline tag. BiTE® antibodies can be designed to exhibit high levels of cytotoxic activity at very low concentrations of 10 to 100 pg/mi and at effector to target cell ratios as low as 2:1.

Antibody V light-chain (VL) and V heavy-chain (VH) domains can be cloned according to standard polymerase chain reaction (PCR) methods. cDNA can be synthesized with oligo dT primers and reverse transcriptase and V domains amplified via PCR with primers 5′L1 and 3′K flanking a VL domain and 5′H1 and 3′G for the heavy chain based on primers described in Dube et al., J. Immunol. Methods 175:89-95 (1994).

VL and \/H regions can be cloned into separate plasmid vectors as templates for a VL- and VH-specific PCR using the oligonucleotide primer pairs 5′VLB5RRV (AGGIGTACAC TCCGATATCC A(ICTGACCCA GTCTCCA)/3′VLGS15 (GGAGCCGCCG CCGCCAGAAC CACCACCACC TTTGATCTCG AGCTTGGTC,C) and 5WHGS15 (GGCGGCGGCG GCTCCGGTGG TGGTGGTTCT CAGGT(GC)(AC)A(AG)C TGCAG(GC)AGTC (AT)GG)/3′VHBspE1 (AATCCGGAGG AGACGGTGAC CGTGGTCCCT IGGCCCCAG). Overlapping complementary sequences can be introduced into the PCR products that combine to form the coding sequence of a 15-amino acid (G₄S₁)₃ linker during the subsequent fusion PCR. This amplification step can be performed with the primer pair 5′VLB5RRV/3′VHBspEa and the resulting fusion product (scF' fragment) can be cleaved with restriction enzymes (e.g. EcoRV and BspEl) and cloned into a plasmid vector (e.g., bluescript KS vector; Stratagene, La Jolla, Calif.) containing the coding sequence of a bispecific single-chain antibody. The resulting bispecific single-chain antibody can be subcloned into an expression vector (e.g., pEF-DHFR) and transfected into CHO cells by, e.g., electroporation and selection. Bispecific antibodies can be purified via its C-terminal Fl tail by affinity chromatography on a nickel-nitrilotriacetic acid (Ni-NTA) column Niagen, linden, Germany) as described in Mack et at., Proc. Natl. Acad. Sci. U.S.A. 92:7021-7025 (1995).

According to another approach described in PCT Publication No. WO 1996/27011, the interface between a pair of antibody molecules can be engineered to maximize the percentage of heterodimers that are recovered from a recombinant cell culture. Such interfaces may comprise at least a part of the Cᵤ domain of an antibody constant domain. By this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodiraers. An alternative method lies two different single chain variable regions to heat stable antigen (HSA). Using HSA as a linker increases serum half-life, and has the benefit of low immunogenicity.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (PCI Publication Nos. WO 1991/100360 and WO 1992/200373 and European Patent No. EP 03089). Heteroconjugate antibodies may be made using any convenient cross-linking methods. Suitable cross-linking agents are well known in the art, and are disclosed in U.S. Pat. No. 4,676,980. along with a number of cross-linking techniques.

Techniques for generating bispecific antibodies from antibody fragments have also been described in the literature. For example, bispecific antibodies can be prepared using chemical linkage. Breunanet et al Science 229:81 (1985) describes a procedure wherein intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab'-thiol by reduction with mercaptoethylamine and is mixed with an equimolar amount of the other Fab′-INB derivative to form the bispecific antibody.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion, The antibody homodimers were reduced at the hinge region to form monomers and then re-oxidized to form the antibody heterodimers. This method can also be utilized for the production of antibody homodimers,

The “diabody” technology described by Hollinger et al., Proc. Nati, Acad. Sci. U.S.A. 90:6444-6448 (1993) provides an alternative mechanism for making bispecific antibody fragments. The fragments comprise a heavy-chain variable domain (V_{H}) connected to a light-chain variable domain (V) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites.

Another strategy for making bispecific antibody fragments by the use of single-chain Fv (sFv) dimers has also been reported. See, Gruber et al., J. Immunol. 152:5368 (1994). Alternatively, antibodies can be “linear antibodies” as described in Zapata et al., Protein Eng. 8(10):1057-1062 (1995). Briefly, these antibodies comprise a pair of tandem Fₐ segments (V_{H}″C_{H1}-V_{H}″C_{H}L) that form a pair of antigen binding regions. Linear antibodies can be bispecific or monospecific.

Within the context of the present disclosure, anti-CD99 antibodies are understood to include monoclonal antibodies and polyclonal antibodies, antibody fragments (e.g., Fab and F(ab′)2), chimeric antibodies, bifunctional or bispecific antibodies and tetrameric antibody complexes.

Antibodies are understood to he reactive against CD99 on the surface of a cell if they bind with an appropriate affinity (association constant), e.g., greater than or equal to 10⁷ M⁻¹. Additionally, antibodies that may be used in the methods of the present disclosure may also be described or specified in terms of their binding affinities include those with a dissociation constant or Kd less than 5×10⁻²M, 10⁻²M, 5×10⁻³M, 10⁻³M, 5×10⁻⁴M, 10⁻⁴M, 5×10⁻⁵M, 10⁻⁵M, 5×10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M.

Antibodies can be fragmented using conventional techniques and the fragments screened for binding activity in the same manner as described above for the whole antibodies. For example, F(ab')2 fragments can be generated by treating antibody with pepsin. The resulting F(ab')2 fragment can be treated to reduce disulfide bridges to produce Fa& fragments.

Chemical conjugation is based on the use of homo- and hetero-bifunctional reagents with E-amino groups or hinge region thiol groups. Homobifunctional reagents such as 5,5′-Dithiobis(2-nitrohenzoic acid) (DNIB) generate disulfide bonds between the two Fabs, and 0-phenylenedimaleimide (O-PDM) generate thioether bonds between the two Fabs. Brenner et al., (1985) and Cilennie et al., (1987). Heterobifunctional reagents such as N-succinimidyl-3-(2-pyridylditio) propionate (SPDP) combine exposed amino groups of antibodies and Fab fragments, regardless of class or isotype. Van Dijk al., (1989).

Peptide or polypeptide linkers can also be used, especially but without limitation those mimicking the hinge region of the Fe domain of antibodies. See, e.g., U.S. Pat. 7,928,072 incorporated in its entirety by reference for disclosure of suitable classes of linkers. The anti-CD99 antibodies of the present disclosure, i.e., antibodies that are useful for treating AML and/or MDS, as well as AML and/or MDS stem cells, such as leukemic stem cells and hematopoietic stem cells, expressing CD99 include derivatives that are modified, i.e., by the covalent attachment of any type of molecule to the antibody such that covalent attachment does not prevent the antibody from binding to its cognate epitope(s) on the extracellular domain of CD99 For example, antibody derivatives can include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques including, but not limited to, specific chemical cleavage, acetylation, formulation, metabolic synthesis of tunicamycin, etc. Additionally, the derivative may contain one or more non-classical amino acids.

Anti-CD99 antibodies according to the present disclosure may be conjugated to one or more cytotoxic compound to further promote antibody cytotoxicity. For example, Antibody Drug Conjugates (ADCs) are a form of hioconjugates/immunoconjugates that are composed of an antibody (a whole rnAb or an antibody fragment such as a single-chain variable fragment [seFv]) linked, via a stable, chemical, linker with labile bonds, to a cytotoxic payload or drug. By combining the CD99-binding and cytotoxic activities of anti-CD99 antibodies with the cancer-killing ability of cytotoxic drugs, antibody-drug conjugates enhance the overall cytotoxic activity and target specificity of the cytotoxic drug. Due to this targeting, the cytotoxin exhibits reduced side effects and the antibody exhibits a further enhanced therapeutic window.

Cytotoxins can be coupled to an anti-CD99 antibody through a stable linkage between the antibody and cytotoxin. Linkers can he based upon chemical motifs including disulfides, hydrazones, or peptides (cleavable), or thioethers (noncleavable) and control the distribution and delivery of the cytotoxic agent to the target cell. Cleavable and noneleavable types of linkers have been proven to be safe in preclinical and clinical trials. For example, Brentuximab vedotin includes an enzyme-sensitive cleavable linker that delivers the potent and highly toxic antimicrotubule agent Monomethyl auristatin E or MMAE, a synthetic antineoplastic agent, to human specific CD3O-positive malignant cells. Because of its high toxicity MMAE, which inhibits cell division by blocking the polymerization of tubulin, cannot be used as a single-agent chemotherapeutic drug. The combination of MMAE linked to an anti-CD3O monoclonal antibody is stable in extracellular fluid, cleavable by cathepsin in vivo, and, ⁻therefore, is safe for therapy. Trastuzumab emtansine, the other approved ADC, is a combination of the microtubule-formation inhibitor mertansin.e (DM-1), a derivative of the Maytansine, and antibody instuzumab (Herceptin®/Genentech/Roche) attached by a stable, non-cleavable linker.

The type of linker, cleavable or noncleticiable, lends specific properties to the cytotoxin. For example, a non-cleavable linker keeps the drug within the cell. As a result, the entire antibody, linker, and cytotoxin can enter the targeted cell where the antibody is degraded to its constituent amino acids. The resulting amino acid, linker and cytotoxin is thereby activated. In contrast, cleavable linkers are enzymatically cleaved in vivo to release the cytotoxin. The cytotoxin can exit the targeted cell and kill neighboring cells.

Detection of Anti-CD99-mediated Aggregation, Clusterinz, and/or Capping of CD99 Expressed on the Surface of AAR. and/or AIDS Cells

Within certain aspects, the present disclosure provides imaging methodology for detecting anti-CD99-mediated aggregation, clustering, and/or capping of CD99 expressed on the surface of .AML and/or MDS cells.

For example, immunofluorescence can be adapted for use for detecting and localizing CD99 on the surface of cells. AML or MDS cells will be incubated with anti-CD99 monoclonal antibodies for three to six hours, followed by fixation with paraformaldehyde and addition of a fluorochrome labeled secondary antibody that binds to the constant domain of the anti-CD99 antibody according to its isotype. Cells will then be mounted onto slides and imaged with confocal or wide-field microscopy. Using this technique, CD99 molecules on the surface of the cells can be assessed for aggregation, clustering, and/or capping with the addition of different anti-CD99 antibodies.

Other methodologies are readily available in the art, which can be adapted for use in detecting anti-CD99-mediated aggregation, clustering, and/or capping of antibody-bound CD99, such as flow cytometric methodologies may be used in confocal-type imaging of antibody bound CD99. For example, Amnis (Seattle, Wash.) offers a high performance system (FlowSightn which includes twelve standard detection channels to simultaneously produce brightfield, darkfield, and ten channels of fluorescence imagery of individual cells.

Detection of Anti-CD99-mediated Cvtotoxicity and Apoptosis of AML and/or MDS Cells Evhibiting Elevated Expression of CD99

Within other aspects, the present disclosure provides methodology for assessing the cytotoxicity of anti-CD99 antibodies in AML and/or MDS cells by detecting anti-CD99-mediated apoptosis ANL and/or MDS cells. By way of example, to test cytotoxicity of anti-CD99 antibodies against AML or MDS cell lines or primary patient samples in vitro, cells can be incubated with anti-CD99 antibodies in media that is either serum-free (for primary patient samples) or containing complement-inactivated sera. For primary patient samples, media can be supplemented with cytokines including stem cell factor, thrombopoietin, 1L-3 and 1L-6, Antibody doses can be titrated over a range from 100 ng/m1 to 20 ng/ml. After 72 hours, the absolute number of viable cells can be enumerated by either manual cell counting with a hemacytometer using trypan blue as a viability exclusion dye, or by single bead-enhanced cylofluorometry (Montes et al., J. Immunol. Methods 317:45 (2006)) using propidium iodide or DAPI as a viability exclusion dye.

To assess for apoptosis in vitro, an experimental set-up similar to that described above can be used, but over a shorter time course (8 to 24 hours). Over this time frame, induction of apoptosis by anti-CD99 antibodies can be assessed by Annexin staining as assessed by flow cytometry, with Annexin V+7-AAD− cells representing early apoptotic cells and Annexin V+7-AAD+ cells representing late apoptotic cells. Apoptotic cells can also be detected by fixation and permeabilization of cells followed by intracellular staining with fluorochrome conjugated antibodies detecting activated caspase 3.

Compositions and Formulations Comprising Anti-CD99 Antibodies

The present disclosure provides compositions, including therapeutic compositions comprising one or more anti-CD99 antibodies for the treatment of AML and/or MDS that is associated with an elevated level of CD99 gene expression. One or more anti-CD99 antibodies can be administered to a human patient as pharmaceutical compositions where each antibody is mixed with a suitable carrier or excipient at doses to treat or ameliorate an AML and/or an MDS as described herein. Mixtures of anti-CD99 antibodies can also be administered to the patient as pharmaceutical compositions.

Compositions within the scope of this disclosure include compositions wherein the therapeutic agent is an anti-CD99 antibody in an amount effective to (1) ligate the extracellular domain of CD99 presented on the surface of a cell that is associated with AML and/or MDS, (2) mediate the clustering and aggregation of the ligated CD99 on AML and/or MDS cells, and (3) promote cytotoxicity of the antibody ligated C1)99 on AML and/or MDS cells by, for example, inducing apoptosis on the antibody ligated cell.

Determination of optimal ranges of effective amounts of each component is within the skill of the art. The effective dose is a function of a number of factors, including the specific anti-CD99 antibody employed as well as the age and clinical status of the patient. The dosage administered will be dependent upon the age, health, and weight of the recipient, kind of concurrent treatment. if any, frequency of treatment, and the nature of the effect desired.

Compositions comprising an anti.-CD99 antibody may be administered parenterally. As used herein, the term “parenteral administration” refers to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid intraspinal, and intrasternal injection and infusion.

Compositions comprising an anti-CD99 antibody may, for example, be administered intravenously via an intravenous push or bolus. Alternatively, compositions comprising an anti-CD99 antibody may be administered via an intravenous infusion.

Suitable dosages for intravenous infusion of a composition comprising an anti-CD99 antibody include a dosage of at least about 2 mg anti-CD99 antibody/m2/day or at least about 10 mg anti-CD99 antibody/m2/day or at least about 20 mg anti-CD99 antibody/m2/day or at least bout 50 mg anti-CD99 antibody/m2/day or at least about 100 mg anti-CD99 antibody/m2/day or at least about 200 mg anti-CD99 antibody/m2/day or at least about 500 mg anti-CD99 antibody/m2/day.

Compositions comprising one or more anti-CD99 antibody may further comprise one or more additional compounds such as, for example, a chemotherapeutic compound for the treatment of AML and/or MDS. For example, induction chemotherapy for AML can include cytarabine (ara-C) and an anthracycline, such as daunorubicin or idarubicin. Cytarabine can be given as a continuous IV infusion for seven consecutive days while the anthracycline is generally given for three consecutive days as an IV push. The AML subtype acute promyelocytic leukemia is treated with all-trans-retinoic acid (ATRA), often combined with an anthracycline and/or Arsenic Trioxide. The presently-disclosed compositions can also include anti-CD99 antibody immunoconjugates crosslinked to a cytotoxic agent, similar to gemtuzumab ozogamicin (Mylotarg).

For MDS, one or more anti-CD99 antibodies may be used in combination with a hematopoietic growth factor (such as erythropoietin) or one of the three agents that have been approved by the U.S. Food and Drug Administration (FDA) for the treatment of MDS, which include 5-azacytidine, decitabine (either alone or in combination with valproic acid), and Lenalidotnide, which is effective in reducing red blood cell transfusion requirement in patients with the chromosome 5q deletion subtype of MDS as well as other low-risk subtypes of MDS, among others.

Compositions comprising an anti-CD99 antibody generally include a therapeutically effective amount of antibody and a pharmaceutically acceptable carrier. As used herein, the term “pharmaceutically acceptable” means approved by a regulatory agency of the Federal or a state government or listed in the U.S, Pharmacopeia. or other generally recognized pharmacopeia for use in animals, and more particularly in humans. he term “carrier” refers to a. diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a. preferred carrier When the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skimmed milk, glycerol, propylene, glycol, water, ethanol and the like. The composition, if desired, can also contain minor amounts of wetting or emulsifying agents, or pH buffering agents.

These compositions can take the form of solutions, suspensions, emulsions, powders, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like. Such compositions will contain a therapeutically effective amount of the anti-CD99 antibody, preferably in purified form, together with a suitable amount of carrier so as to provide the form for proper administration to the patient. The formulation should suit the mode of administration.

Compositions can be formulated in accordance with routine procedures as a pharmaceutical composition adapted for intravenous administration to a human. Typically, compositions for intravenous administration are solutions in sterile isotonic aqueous buffer or other physiologically acceptable buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion battle containing sterile pharmaceutical grade water or saline. Where the composition is administered by injection, an ampoule of sterile water fix injection or saline can be provided so that the ingredients may be mixed prior to administration.

The anti-CD99 antibodies disclosed herein can be formulated as neutral or salt forms. Pharmaceutically acceptable salts include those farmed with anions such as those derived from hydrochloric, phosphoric, acetic, oxalic, tartaric acids, and the like, and those formed with cations such as those derived from sodium, potassium, ammonium, calcium, ferric hydroxides, isopropylamine, triethylamine, 2-ethylamino ethanol, histidine, procaine, and the like,

Many of the anti-CD99 antibodies of the present disclosure may be provided as salts with pharmaceutically compatible counterions (i.e., pharmaceutically acceptable salts). A “pharmaceutically acceptable salt” means any non-toxic salt that, upon administration to a recipient, is capable of providing, either directly or indirectly, an antibody or composition of this disclosure,

A “pharmaceutically acceptable counterion” is an ionic portion of a salt that is not toxic when released from the salt upon administration to a subject. Pharmaceutically compatible salts may be formed with many acids, including but not limited to hydrochloric, sulfuric, acetic, lactic, tartaric, malic, and succinic acids, Salts tend to be more soluble in water or other protic solvents than their corresponding free base forms. The compositions of the present disclosures may include such salts.

Compositions according to the present disclosure can be used ex vivo, for example to purge autologous bone marrow of CD99+ tumor cells (and CD99+ tumor stem cells), following known methods. See, e.g., Robertson et ai., 79:2229-2236 (1992).

Methodology for Detecting Elevated CD99 Expression on AML and MDS Cell

The present disclosure provides antibodies, and compositions thereof, as well as methods that employ one or more antibody or antibody compositions that exhibit cytotoxicity promoting and/or apoptosis inducing activity against AML and/or MDS tissues or cells that exhibiting elevated CD99 cell surface expression. Thus, the present disclosure provides methodologies for assessing elevated CD99 expression which rely upon the quantification of the extent of CD99 gene expression, including the relative activity of CD99 gene transcription and levels of CD99 transcripts, as well as the relative number of CD99 protein molecules exposed on the surface of AML and/or MDS tissues or cells. Exemplary methodologies for assessing elevated. CD99 expression are presented in the present section.

Elevated CD99 gene expression can be determined by one or more methodologies that are well known in the art including, for example, microarray, quantitative PCR, including real-time-PCR (RT-PCR), and direct RNA sequencing Each of the methodologies described herein for the detection of elevated CD99 gene expression has in common the detection of a CD99 polynucleotide via the amplification, hybridization. and/or sequencing of mRNA encoded by a CD99 gene.

As used herein, the term “elevated gene expression,” in particular the term “elevated CD99 gene expression,” refers to a level of gene expression that is at least about two-fold at least about three-fold, at least about five-fold at least about 10-fold or greater in an AML and/or MDS tissue sample or cell as compared to a control tissue or cell, which can be an internal or an external control tissue or cell.

As used herein, the term “internal control” refers to a nucleotide sequence, typically a gene or genetic sequence. which does not exhibit elevated expression in an AML and/or MDS tissue or cell as compared to a non-AML and/or a non-MDS tissue or cell. Thus, for example, an “internal control” can be used as a “negative control” for assessing whether a CD99 gene exhibits elevated expression levels in an AML and/or MDS tissue sample or cell without reference to a non-leukemia tissue sample or cell.

Suitable genes that can serve as “internal controls” include, for example and without limitation, β-actin, (IAPDH, and cyclophilin. The levels of CD99 gene expression and internal control gene expression (i.e., non-CD99 gene expression) can be determined (e.g., by quantifying the number of CD99 transcripts), a ratio of CD99 and non-CD99 gene expression can be derived, and the level of CD99 gene expression within a given an AML and/or MDS tissue sample or cell can be expressed in terms of the ratio of CD99 and non CD99 gene expression, wherein a ratio greater than a pre-determined threshold ratio indicates elevated CD99 gene expression.

In contrast, as used herein, the term “external control” refers to a CD99 gene or genetic sequence from a non-ANIL and/or non-MDS tissue or cell, which CD99 gene or genetic sequence does not exhibit elevated expression in the non-AML and/or non-MDS tissue or cell but is being tested for elevated expression in a corresponding AML and/or MDS tissue or cell Thus, for example, an “external control” can be used as a “negative control” for assessing whether the CD99 gene exhibits elevated expression levels in an AML and/or MDS tissue sample or cell by comparing the level of expression (e.g., the number of mRNA transcripts) in an AML and/or MDS tissue sample or cell to a corresponding non-AML and/or non-MDS tissue sample, such as a tissue sample from a normal donor, or non-AML and/or non-MDS cell, such as a CD34⁺ non-AML and/or non-MDS cell.

Elevated CD99 gene expression can also be assessed on the basis of the percentage or fraction of blasts (i.e., AML and/or MDS cells) relative to the total number of cells in a given tissue sample from an AML and/or MDS patient. By this methodology, for example, the number of CD99-associated transcripts in an AML and/or MDS tissue sample can be quantified and multiplied by the inverse percentage or fraction of blasts in the AML and/or MDS tissue sample. The resulting CD99 transcript number can then be assessed relative to a threshold transcript number for CD99 gene expression and, based upon that assessment, the responsiveness of an ANIL, and/or MDS patient to a therapeutic regimen comprising the administration of an anti-GD99 antibody, or derivative thereof, can be predicted. More specifically, by this methodology, a transcript number for CD99 gene expression that is greater than a threshold transcript number would be predictive of the therapeutic efficacy of such a treatment regimen.

Measurement of elevated CD99 gene expression can, for example, be accomplished by (1) quantifying a CD99 mRNA in a tissue sample from an AML and/or a MDS patient; (2) quantifying the level of the CD99 mnRNA in a tissue sample from a non-AML and/or a non-MDS control donor; and (3) comparing the level of the CD99 mRNA in the tissue sample from the AML and/or MDS patient with the level of the CD99 mRNA in the tissue sample from the control donor. It will be understood that an elevated level of CD99 mRNA in the AML and/or MDS patient tissue sample as compared to CD99 mRNA in the control donor tissue sample indicates the susceptibility of the AML and/or MDS patient to treatment with an anti-CD99 antibody exhibiting the structural and/or functional properties described herein.

Alternatively, elevated CD99 gene expression can be tested by (1) quantifying CD99 mRNA levels in a tissue sample from an AML and/or MDS patient; (2) quantifying the level of a non-CD99 mRNA in the AML and/or MDS patient tissue sample, such as, for example, GAPDH or actin; and (3) comparing the level of the CD99 mRNA in the tissue sample from the AML and/or MDS patient with the level of the non-CD99 mRNA in the AML and/or MDS patient tissue sample. h will be understood that an elevated level of the CD99 mRNA in the AML andlor MDS patient tissue sample as compared to the non-CD99 mRNA in the AML and/or MDS patient tissue sample indicates the susceptibility of the AML and/or MDS patient to treatment with an anti-CD99 antibody as disclosed herein.

Within certain aspects of these methods a CD99 mRNA can be quantified by amplifying mRNA in a tissue sample, whether a AML and/or MDS patient tissue sample or cell, a non-A.ML and/or MDS tissue sample or cell from a AML and/or MDS patient, or a tissue sample or cell from a non-AML and/or MDS control donor, with a primer pair that is specific for CD99 mRNA (see Table 2A). Likewise, a non-CD99 mRNA can be quantified by amplifying RNA in a tissue sample, whether a AML, and/or MDS patient tissue sample or cell, a non-AML, and/or MDS tissue sample or cell from a AML and/or MDS patient, or a tissue sample or cell from a non-ANIL andlor MDS control donor, with a primer pair that is specific for a non-CD99 mRNA. A primer pair comprises a forward primer and a reverse primer, wherein the forward primer hybridizes toward the 5′ end of an mRNA and wherein said reverse primer hybridizes toward the 3′ end of the mRNA, whether the mRNA is a CD99 RNA or a non-CD99 mRNA.

Examples of nucleotide sequences for mRNA encoded by CD99 genes are presented in Table 2A, as are the corresponding accession numbers and sequence identifiers.

TABLE 2A
Sequences Encoded by the Human CD99 Gene

| H. sapiens CD99 mRNA, | | |
|---|---|---|
| Accession Number | | |
| Sequence Identifier | Nucleotide Sequence Amino Acid Sequence | |
| CD99 (Variant 1) | 1 | ggaggccggg gcggggcggg cgcagccggc gctgagcttg cagggccgct cccctcaccc |
| NCBI: NM_0024143 | 61 | gcccccttcg agtccccggg cttcgcccca cccggcccgt gggggagtat ctgtcctgcc |
| SEQ ID NO: 1 | 121 | gccttcgccc acgccctgca ctccgggacc gtccctgcgc gctctgggcg caccatggcc |
| | 181 | cgcggggctg cgctggcgct gctgctcttc ggcctgctgg gtgttctggt cgccgcccog |
| | 241 | gatggtggtt tcgatttatc cgataccctt cctgacaatg aaaacaagaa acccactgca |
| | 301 | atccccaaga aacccagtgc tggggatgac tttgacttag gagatgctgt tgttgatgga |
| | 361 | gaaaatgacg acccacgacc accgaaccca cccaaaccga tgccaaatcc aaaccccaac |
| | 421 | caccctagtt cctccggtag cttttcagat gctgaccttg cggatggcgt ttcaggtgga |
| | 481 | gaaggaaaag gaggcagtga tggtggaggc agccacagga aagaagggga agaggccgac |
| | 541 | gccccaggcg tgatccccgg gattgtgggg gctgtcgtgg tcgccgtggc tggagccatc |
| | 601 | tctagcttca ttgcttacca gaaaaagaag ctatgcttca aagaaaatgc agaacaaggg |
| | 661 | gaggtggaca tggagagcca ccgaaatgcc aacgcagagc cagctgttca gcgtactctt |
| | 721 | ttagagaaat agaagattgt cggcagaaac agcccaggcg ttggcagcag ggttagaaca |
| | 781 | gctgcctgag gctcctccct gaaggacacc tgcctgagag cagagatgga ggccttctgt |
| | 841 | tcacggcgga ttctttgttt taatcttgcg atgtgctttg cttgttgctg ggcggatgat |
| | 901 | gtttactaac gatgaatttt acatccaaag ggggataggc acttggaccc ccattctcca |
| | 961 | aggcccgggg gggcggtttc ccatgggatg tgaaaggctg gccattatta agtccctgta |
| | 1071 | actcaaatgt caaccccacc gaggcacccc cccgtccccc agaatcttgg ctgtttacaa |
| | 1081 | atcacgtgtc catcgagcac gtctgaaacc cctggtagcc ccgacttctt tttaattaaa |
| | 1141 | ataaggtaag cccttcaatt tgtttcttca atatttcttt catttgtagg gatatttgtt |
| | 1201 | tttcatatca gactaataaa aagaaattag aaaccaaaaa aaaaaaaaaa aaaaa |
| CD99 (Variant 1) | MARGAALALL LFGLLGVLVA APDGGFDLSD ALPDNENKKP TAIPKKPSAG DDFDLGDAVV DGENDDPRPP | |
| NCBI: NM_002414.3 | NPPKPMPNPN PNHPSSSGSF SDADLADGVS GGEGKGGSDG GGSHRKEGEE ADAPGVIPGI VGAVVVAVAG | |
| SEQ ID NO: 2 | AISSFIAYQK KKLCFKENAE QGEVDMESHR NANAEPAVQR TLLEK | |
| CD99 (Variant 2) | 1 | ggaggccggg gcggggcggg cgcagccggc gctgagcttg cagggccgct cccctcaccc |
| NCBI: NM_001122898.1 | 61 | gcccccttcg agtccccggg cttcgcccca cccggcccgt gggggagtat ctgtcctgcc |
| SEQ ID NO: 3 | 121 | gccttcgccc acgccctgca ctccgggacc gtccctgcgc gctctgggcg caccatggcc |
| | 181 | cgcggggctg cgctggcgct gctgctcttc ggcctgctgg gtgttctggt cgccgccccg |
| | 241 | gatggtggtt tcgatttatc cgataccctt cctggggatg actttgactt aggagatgct |
| | 301 | gttgttgatg gagaaaatga cgacccacga ccaccgaacc cacccaaacc gatgccaaat |
| | 361 | ccaaacccca accaccctag ttcctccggt agcttttcag atgctgacct tgcggatggc |
| | 421 | gtttcaggtg gagaaggaaa aggaggcagt gatggtggag gcagccacag gaaagaaggg |
| | 481 | gaagaggccg acgccccagg cgtgatcccc gggattgtgg gggctgtcgt ggtcgccgtg |
| | 541 | actggagcca tctctagctt cattacttac cagaaaaaga agctatgctt caaagaaaat |
| | 601 | gcagaacaag gaaaggtgga catggagagc caccggaatg ccaacgcaga gccagctgtt |
| | 661 | cagcgtactc ttttagagaa atagaagatt gtcggcagaa acagcccagg cgttggcagc |
| | 721 | agggttagaa cagctgcctg aggctcctcc ctgaaggaca cctgcctgag agcagagatg |
| | 781 | gaggccttct gttcacggcg gattctttgt tttaatcttg cgatgtgctt tgcttgttgc |
| | 841 | tgggcggatg atgtttacta acgatgaatt ttacatccaa agggggatag gcacttggac |
| | 901 | ccccattctc caaagcccgg gggggcggtt tcccatggga tgtgaaaggc tggccattat |
| | 961 | taagtccctg taactcaaat gtcaacccca ccgaggcacc cccccgtccc ccagaatctt |
| | 1021 | ggctgtttac aaatcacgtg tccatcgagc acgtctgaaa cccctggtag ccccgacttc |
| | 1081 | tttttaatta aaataaggta agcccttcaa tttgtttctt caatatttct ttcatttgta |
| | 1141 | gggatatttg tttttcatat cagactaata aaaagaaatt agaaaccaaa aaaaaaaaaa |
| | 1201 | aaaaaaa |
| CD99 (Variant 2) | MARGAALALL LFGLLGVLVA APDGGFDLSD ALPGDDFDLG DAVVDGENDD PRPPNPPKPM PNPNPNHPSS | |
| NCBI: NM_001122898.1 | SGSFSDADLA DGVSGGEGKG GSDGGGSHRK EGEEADAPGV IPGIVGAVVV AVAGAISSFI AYQKKKLCFK | |
| SEQ ID NO: 4 | ENAEQGEVDM ESHRNANAEP AVQRTLLEK | |
| CD99 (Variant 3) | 1 | ggaggccggg gcggggcggg cgcagccggc gctgagcttg cagggccgct cccctcaccc |
| NCBI: NM_001277710.1 | 61 | acccccttcg agtccccggg cttcacccca cccggcccgt gggggagtat ctgtcctgcc |
| SEQ ID NO: 5 | 121 | gccttcgccc acgccctgca ctccgggacc gtccctgcgc gctctgggcg caccatggcc |
| | 181 | cgcggggctg cgctggcgct gctgctcttc ggcctgctgg gtgttctggt cgccgccccg |
| | 241 | gatggtggtt tcgatttatc cgatgccctt cctgacaatg aaaaaaaaaa acccactgca |
| | 301 | atccccaaga aacccagtgc tggggatgac tttgacttag gagatgctgt tgttgatgga |
| | 361 | aaaaatgacg acccacgacc accgaaccca cccaaaccga tgccaaatcc aaaccccaac |
| | 421 | caccctagtt cctccggtag cttttcagat gctgaccttg cggatggcgt ttcaggtgga |
| | 481 | gaaggaaaag gaggcagtga tggtggaggc agccacagga aagaagggga agaggccgac |
| | 541 | gccccaggcg tgatccccgg gattgtgggg gctgtcgtgg tcgccgtggc tggagccatc |
| | 601 | tctagcttca ttgcttacca gaaaaagaag ctatgcttca aagaaaatga tggctgaaga |
| | 661 | cctagggaac aaggggaggt ggacatggag agccaccgga atgccaacgc agagccagct |
| | 721 | attcagcgta ctcttttaga gaaataaaag attgtcggca gaaacagccc aggcgttggc |
| | 781 | agcagggtta gaacagctgc ctgaggctcc tccctgaagg acacctgcct gagagcagag |
| | 841 | atggaggcct tctgttcacg gcggattctt tgttttaatc ttgcgatgtg ctttgcttgt |
| | 901 | tgctgggcgg atgatgttta ctaacgatga attttacatc caaaggggga taggcacttg |
| | 961 | gacccccatt ctccaaggcc cgggggggcg gtttcccatg ggatgtgaaa ggctggccat |
| | 1021 | tattaagtcc ctgtaactca aatatcaacc ccaccgaggc acccccccgt cccccagaat |
| | 1081 | cttggctgtt tacaaatcac gtgtccatcg agcacgtctg aaacccctgg tagccccgac |
| | 1141 | ttctttttaa ttaaaataag gtaagccctt caatttgttt cttcaatatt tctttcattt |
| | 1201 | gtagggatat ttgtttttca tatcagacta ataaaaagaa attagaaacc aaaaaaaaaa |
| | 1261 | aaaaaaaaaa |

| | |
|---|---|
| CD99 (Variant 3) | MARGAALALL LFGLLGVLVA APDGGFDLSD ALPDNENKEP TAIPKKPSAG DDFDLGDAVV DGENDDPRPP |
| NCBI: NM_001277710.1 | NPPKPMPNPN PNHPSSSGSF SDADLADGVS GGEGKGGSDG GGSHRKEGEE ADAPGVIPGI VGAVVVAVAG |
| SEQ ID NO: 6 | AISSFIAYQK KKLCFKENDG |
| extracellular domain | MARGAALAAL LFGLLGVLVA APDGGFDLSD ALPDNENKKP TAIPKKPSAG DDFDLGDAVV |
| of CD99 | DGENDDPRPP NPPKPMPNPN PNHPSSSGSF SDADLADGVS GGEGKGGSDG GGSHRKEGEE |
| SEQ ID NO: 15 | ADAPG |

In order to identify an AML and/or MDS patient tissue sample or cell that has elevated CD99 gene expression, mRNA can be isolated from an AML and/or MDS patient tissue sample or cell and from a non-AML and/or MDS control tissue sample or cell, the level of expression of a given mRNA can be determined and an assessment of elevated gene expression can be made by comparing the mRNA levels determined for an AML and/or MDS patient tissue sample or cell and a non-AML and/or MDS control tissue sample or cell.

Alternatively, an AML and/or MDS patient tissue sample or cell that has elevated CD99 gene expression can be identified by isolating mRNA from an AML and/or MDS patient tissue sample or cell, determining the levels of a CD99 mRNA and a control mRNA, and assessing elevated gene expression by comparing the CD99 mRNA and control mRNA levels within the leukemia tissue sample or cell to determine the ratio of mRNA expression, wherein an elevated ratio of CD99 mRNA level relative to control mRNA level indicates an elevated level of CD99 gene expression. As used in this context, a control mRNA refers to an mRNA from a gene that does not exhibit an elevated level of expression in an AML and/or MDS tissue or cell. Suitable control mRNAs include, for example, β-actin., GAPDH, and cyclophilin,

Suitable AML and/or MDS tissue samples include, for example, blood, lymph node, bone marrow, and/or tumor biopsy samples from an AML and/or MDS patient. Suitable non-AML and/or MDS control tissue samples include, for example, blood, lymph node, and/or bone marrow samples from a non-A.ML and/or MDS donor, such as a healthy, disease-free donor. Such blood, lymph node, and/or bone marrow samples from a non-AML and/or MDS donor typically contain CD34⁺ cells. It will be understood that, regardless of the precise nature or source of the donor tissue sample or cell, it is essential that the donor tissue or cell is known not to exhibit elevated expression of the CD99 gene.

Suitable AML and/or MDS cells include, for example, myeloid precursors. Suitable non-AML and/or MDS control cells, in particular non-AML and/or MDS CD34⁺ control cells, include, for example, myeloid precursors from a non-AML and/or MDS donor, such as a healthy, disease-free donor or one or more cell line, such as a CD34′ cell line including, for example, thelKasumi-1 cell line. Regardless of its source or identity, it will be understood that a suitable non-AML andlor MDS control tissue sample or cell will be characterized by not exhibiting elevated levels of CD99 gene.

### AML Cell Lines

Nearly all AML cell lines tested were responsive to anti-CD99 MAbs. In some embodiments, one or more AML cell lines are employed in the methods of the disclosure, wherein the one or more cell lines are selected from the group consisting of Kg1a. SET2, Mega1a, THP-1, NB4, AML14, KBM5, AML5Q, KCL22, MOLM14, HL60, K562, U937, KU812, MOLM13, Monomac, and NOMO1 .

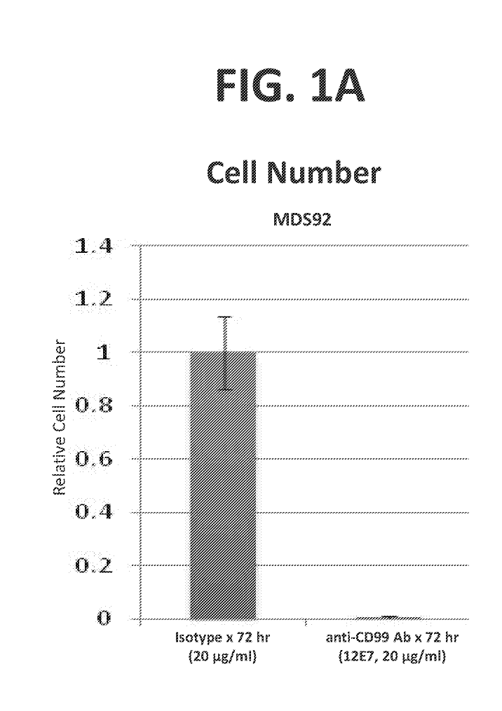

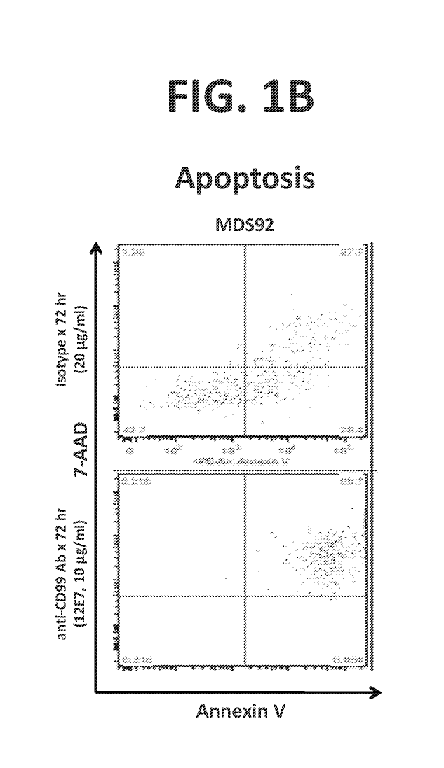

FIG. 1.0A is a bar graph showing the relative cell number for 15 AML cell lines tested for cell growth inhibition following 48 hr incubation with anti-CD99 antibody 12E7. Eleven of the 15 AML cell lines tested, AML 5Q, ⁻NB4, KCL22, MOLM13, HL60, /MOLM14, NOMO1, U937, MonoMacl, KG1a, and AML 14 were found to be susceptible to growth inhibition following CD99 ligation with 12E7 antibody. Four cell lines tested, KBM5, SET2, K11812, and K562 were characterized as unresponsive to CD99 ligation with 12E7 antibody.

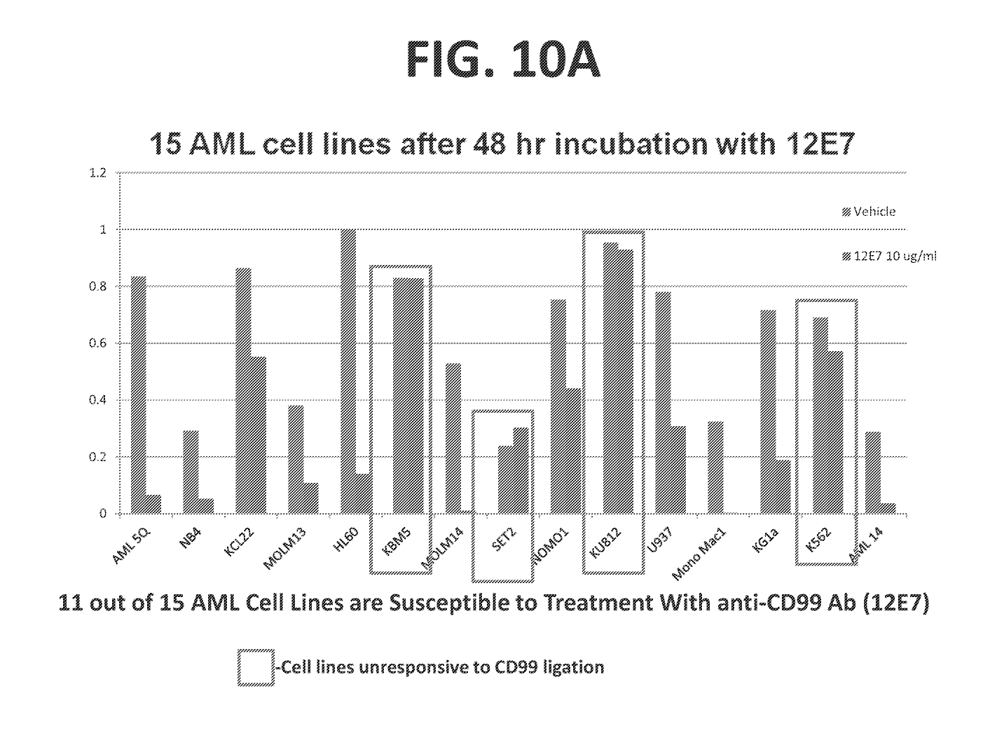

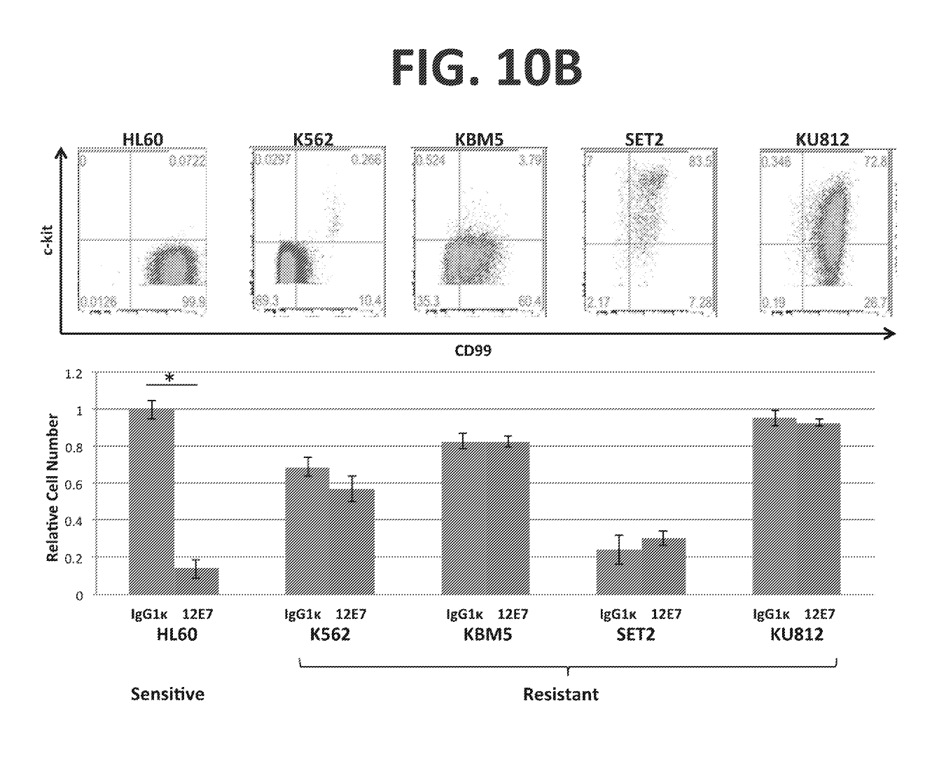

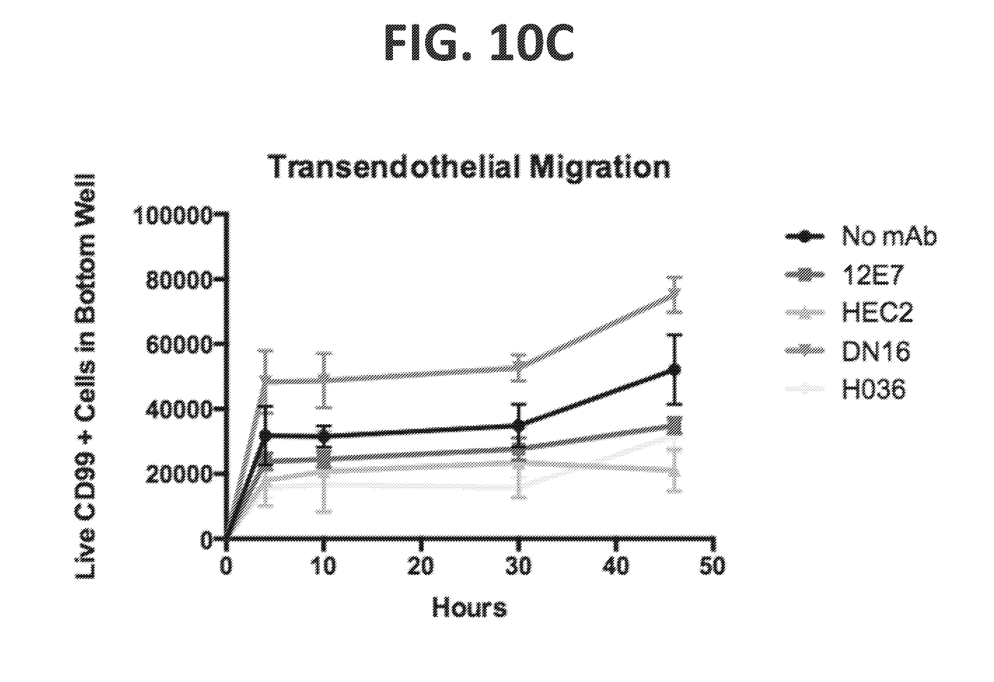

FIG. 10B shows flow cytometry plots showing relative expression of c-kit and CD99 in AML cell lines HL60, K562, KBM5, SET2, and KU812 cells (top panel) and bar graphs showing relative cell number after 48 hour incubation withligEilk or anti-CD99 12E7 antibody (bottom panel). HL60 is a representative “sensitive” cell line, with high CD99 and low c-kit expression. “Resistant” cell lines either do not express CD99 (K562 and KBM5), express high levels of c-kit (SEM2 and K1J⁻812), are Bcr-abl positive (K562/KBM5/KU812), or are JAK2 positive (SEM2). These data demonstrate that low CD99 expression or high c-kit expression are predictive for resistance to cytotoxicity mediated by anti-CD99 antibody 12E7.

FIG. 10C is a graph of transendothelial migration kinetics of CD99+AML cells in the presence of anti-CD99 antibodies 12E7, HEC2, DN16, and H036, indicating that in the absence of secondary antibody, certain anti-CD99 antibodies do not inhibit transendothelial migration of AML cells, which is known to be mediated by CD99.

In a separate experiment, 17 AML cell lines were exposed to anti-CD99 MAb H036-1.1. All 17 AML cell lines demonstrated response to anti-CD99 monoclonal antibody H036-1,1 and for most (11/17) the response was complete at a dosage tested. The decrease in cell number after antibody exposure ranged from 2.74 fold to 370.5 fold as shown in Table

TABLE 2B
AML Cell Lines Responsive to anti-CD99 MAb H036-1.1.

| | | Fold decrease in cell |
|---|---|---|
| | | number at log(H036- |
| AML Cell line | IC50 (ng/mL) | 1.1) = 4 |
| Kg1a | 456.3 | 5.35 |
| SET2 | 320.7 | 3.58 |
| Meg1a | 109.7 | 8.36 |
| THP-1 | 357.9 | 21.9 |
| NB4 | 404.2 | 27.1 |
| AML14 | 491.0 | 20.0 |
| KBM5 | 188.5 | 29.9 |
| AML5Q | 256.7 | 52.2 |
| KCL22 | 227.8 | 3.90 |
| MOLM14 | 38.58 | 120 |
| HL60 | 370.5 | 71.9 |
| K562 | 125.9 | 2.74 |
| U937 | 238 | 19.8 |
| KU812 | 369.7 | 37.4 |
| MOLM13 | 358.6 | 21.3 |
| Monomac | 485.3 | 47.9 |
| NOMO1 | 391 | 13.7 |

In this experiment, all 17 AML cell lines tested, Kgla, SET2, Meg1a, THP-1, N34, AML14, KBM5, AML5Q, KCL22, MOLM14, HL60, K562, U937, KU812, MOLM13, Monomac, and NOM01 were found to be susceptible to growth inhibition following CD99 ligation with anti-CD99 H036-1.1 antibody (Abeam). Four cell lines tested, Kg la, SE12, KCL22, and K562 were found to be partially responsive to growth inhibition by CD99 ligation with H036-1.1 antibody.

Methodologies for quantifying gene expression levels that can be readily adapted to detecting elevated expression of CD99 genes are now described in further detail.

Microarry Analysis

Elevated CD99 gene expression can he detected and quantified by microarray analysis of RNA isolated from an AML and/or MDS patient and/or control donor tissue sample- or cell. Due to limitations on its sensitivity, however, microarray methodology may not accurately determine the absolute tissue distribution of low abundance genes or may underestimate the degree of elevated CD99 gene expression due to signal saturation. For those cells showing elevated CD99 expression by microarray expression profiling, further analysis can be performed using one or more quantitative PCR methodology such as, for example, RT-PCR based on Taqman^{ilm} ₚrobe detection (Invitrotten Life Sciences, Carlsbad, CA), which provides a greater dynamic range of sensitivity.

Briefly, microarray analysis includes that PCR amplification of RNA extracted from an AML and/or MDS patient or control donor tissue sample or cell with primer pairs that hybridize to coding sequences within a CD99 gene and/or coding sequences within a non-CD99 gene the expression of which is to be detected and/or quantified. PCR products are dotted onto slides in an array format, with each PCR product occupying a unique location in the an⁻ay. The RNA is then reverse transcribed and fluorescent-labeled cDNA probes are generated. Microarrays are probed with the fluorescent-labeled cDNA probes, slides are scanned, and fluorescence intensity is measured. The level of fluorescence intensity correlates with hybridization intensity, which correlates with relative level of gene expression.

CD99 gene expression analysis can be performed using a commercially available microarray (e.g., the U133A chip; Affymetrix, Santa Clara, Calif.) or using a custom microarray. Alternatively, elevated CD99 gene expression can be detected using a Synteni microarray (Palo Alto, Calif.) according to the manufacturer's instructions and as described by Schena et al., Proc. Natl. Acad. Sci. US.A. 93:10614-10619 (1996) and Heller et al., Proc. Natl. Acad. Sci. US.A. 94:2150-2155 (1997). Microarray hybridization can be performed according to methodology described in Abraham et al., Blood 105:794-803 (2005).

Probe level data can be normalized using a commercial algorithm (e.g., the Affymetrix Microarray Suite 5.0 algorithm) or a custom algorithm. CD99 gene expression intensity values as well as non-CD99 gene expression intensity values can be log transformed, median centered, and/or analyzed using commercially available programs (e.g, GeneSpring 7.3.1 GX; Agilent Technologies, Santa Clara, Calif.) or a custom algorithm.

A number of factors can be used to assess the quality of the CD99 gene expression analysis such as, for example, the GAPDH 3′:5′ ratio and the actin 3′:5′ ratio. Samples with poor quality results can be defined as having a GAPDH 3′:5′ ratio of greater than about 1.25 and/or an actin 3′:5′ ratio of greater than about 3.0.

Elevated CD99 gene expression can be determined using Welch's ANOVA using variance computed by applying the cross-gene error model based on deviation from 1 available within GeneSpring. This can overcome a lack of replicates and variance associated with the individual samples and can be considered to be similar in principle to variance filtering. Unsupervised clustering can be done using a hierarchical agglomerative algorithm. Pearson's correlation coefficient and centroid linkage can be used as similarity and linkage methods, respectively.

To detect possible differences between samples, genes can be extracted from the dataset that had 1.5-fold difference in expression between individual samples and/or were statistically significant at a corrected P value of 0.05 by Student's t test with Benjamini-Hochberg multiple testing corrections. Differentially expressed genes can be assessed for Gene Ontology (GO) enrichment (e.g., using GeneSpring),

Quantitative PER

Depending upon such factors as the relative number of AML and/or MDS cells present in an AML and/or MDS tissue sample and/or the level of CD99 gene expression within each AML and/or MDS cell within a tissue sample, it may be preferred to perform a quantitative PER analysis to detect and/or quantify the level of CD99 gene expression.

For example, at least two oligonucleotide primers can be employed in a PER-based assay to amplify at least a portion of a CD99 gene mRNA and/or a non-HOX cluster/non-CD99 gene mRNA, or a corresponding eDNA, which is derived from an AML and/or MDS tissue sample or cell and/or a non-AML and/or MDS control donor tissue sample or cell. At least one of the oligonucleotide primers is specific for, and hybridizes to, a mRNA that is encoded by a. CD99 gene. The amplified cDNA may, optionally, be subjected to a fractionation step such as, for example, get electrophoresis prior to detection.

RT-PCR is a quantitative PER methodology in which PER amplification is performed in conjunction with reverse transcription. RNA is extracted from a tissue sample or cell, such as a blood, lymph node, bone marrow, and/or tumor biopsy sample, and is reverse transcribed to produce eDNA molecules. PER amplification using at least one specific primer amplify the cDNA molecule, which may be separated and visualized using, for example, gel electrophoresis. Amplification may be performed on tissue samples or cells taken from a patient and from a control who is not afflicted with a cancer. The amplification reaction may be performed on several dilutions of cDNA spanning two orders of magnitude. An increase in expression of at least about three-fold, at least about five-fold, at least about 10-fold, at least about 15-fold, at least about 20-fold, at least about 25-fold, or greater in several dilutions of the test an AML and/or MDS patient sample as compared to the same dilutions of the non-AML and/or MDS control donor sample is typically considered positive.

As used herein, the term “amplification” refers to the production of multiple copies of a target nucleic acid that contains at least a portion of the intended specific target nucleic acid sequence. The multiple copies are referred to, interchangeably, as amplicons or amplification products. In certain aspects of the present disclosure, the amplified target contains less than the complete target mRNA sequence (i.e., spliced transcript of exons and flanking untranslated sequences) and/or target genomic sequence (including introns andlor exons). For example, specific amplicons may be produced by amplifying a portion of the target polynucleotide by using amplification primers that hybridize to, and initiate polymerization from, internal positions of the target polynucleotide. The amplified portion contains a detectable target sequence that may be detected using any of a variety of well-known methods.

Many welt-known methods of nucleic acid amplification require thermoeyc ing to alternately denature double-stranded nucleic acids and hybridize primers; however, other well-known methods of nucleic acid amplification are isothermal. The polymerase chain reaction (PCR; described in U.S. Pat. Nos. 4,683,195; 4,683.202; 4,800,159; 4,965,188) uses multiple cycles of denaturation, annealing of primer pairs ⁻to opposite strands, and primer extension to exponentially increase copy numbers of the target sequence. In a variation called RT-PCR, reverse transcriptase (RT) is used to make a complementary DNA (cDNA) from mRNA, and the cDNA is then amplified by PCR to produce multiple copies of DNA.

CD99 gene expression may be further characterized or, alternatively, originally detected and/or quantified by employing the quantitative real-time PCR methodology. Gibson et al., Genome Research 6:995-1001 (1996) and Heid et al., Genome Research 6:986-994 (1996). Real-time PCR is a technique that evaluates the level of PCR product accumulation during the course of amplification. This technique permits quantitative evaluation of mRNA levels in multiple samples, By this methodology, an AML and/or MDS tissue sample or cell may be tested along-side a corresponding non-AML and/or MDS control donor sample or cell andior a panel of unrelated normal non-AML and/or MDS tissue samples or cells.

Real-time PCR may, for example, be performed either on the AIB 7700 Prism or on a Gene.Amp,R™ 5700 sequence detection system (Applied Biosystems, Foster City, Calif.). The 7700 system uses a forward and a reverse primer in combination with a specific probe with a 5 fluorescent reporter dye at one end and a 3′ quencher dye at the other end (Taqinan^{fm}). When real-time PCR is performed using Taq DNA polymerase with 5′-3′ nuclease activity, the probe is cleaved and begins to fluoresce allowing the reaction to be monitored by the increase in fluorescence (real-time). The 5700 system uses SYBR®green, a fluorescent dye that only binds to double stranded DNA, and the same forward and reverse primers as the 7700 instrument. Matching primers and fluorescent probes may be designed according to the primer express program (Applied Biosystems, Foster City, Calif.). Optimal concentrations of primers and probes are initially determined by those of ordinary skill in the art. Control (e.g., β-actin-specific) primers and probes may be obtained commercially from, for example, Perkin Elmer/Applied Biosystems (Foster City, Calif.),

To quantify the amount of CD99 gene expression in a sample, a standard curve is generated using a plasmid containing the gene of interest. Standard curves are generated using the Ct values determined in the real-time PCR, which are related to the initial eDNA concentration used in the assay. Standard dilutions ranging from 10-10° copies of the gene of interest are generally sufficient. In addition, a standard curve is generated for the control sample sequence. This permits standardization of initial RNA content of an AML and/or MDS tissue sample or cell to the amount of a control tissue sample or cell for comparison purposes.

Total RNA may be extracted from AML and/or MDS tissue samples or cells and non-AML and/or MDS control tissue samples or cells using Trizol reagent as described herein. First strand synthesis may be carried out using 1-2 μg of total RNA with SuperScript II reverse transcriptase (Lifi. Technologies, Carlsbad, Calif.) at 42° C. for one hour. cDNA may then be amplified by PCR with CD99 gene-specific primers that are designed based upon the CD99 mRNA or other sequences presented in Table 2A or that are otherwise known and readily available to those skilled in the art,

To ensure the quantitative nature of the RT-PCR, a housekeeping gene, such as β-actin, can be used as an internal control for each of the AML and/or MDS patient and non-AML and/or MDS control donor tissue samples andlor cells examined. Serial dilutions of the first strand cDNAs are prepared and RT-PCR assays are performed using Il-actin specific primers. A dilution is then chosen that enables the linear range amplification of the β-actin template and that is sensitive enough to reflect the differences in the initial copy numbers. Using these conditions, the -actin levels are determined for each reverse transcription reaction from each tissue, DNA contamination is minimized by DNase treatment and by assuring a negative PCR result when using first strand cDNA that was prepared without adding reverse transcriptase.

In an exemplary RT-PCR reaction using the Dynabeads mRNA direct microkit (Invitrogen, Life Sciences Technologies, Carlsbad, Calif.), samples containing 10⁵ cells or less are tested in a total reaction volume of 30 μl with 14.25 μl H₂O; 1.5 μl BSA; 6 μl first strand buffer; 0.75 mL of 10 mM dNTP mix; 3 μl Rnasin; 3 0.1 M HT; and 1.5 pl Superscript The resulting solution is incubated for 1 hour at 42° C., diluted 1:5 in H₂O, heated at 80° C. for 2 min to detach cDNA from the beads, and immediately placed on MPS. The supernatant containing cDNA is transferred to a. new tube and stored at −20° C.

RNA Sequencing

Elevated expression of a CD99 gene can be determined by the direct sequencing of mRNA in an AML and/or MDS patient tissue sample or cell and/or non-AML andlor MDS donor control tissue sample or cell. Alternatively, elevated expression of the CD99 gene can be determined fallowing conversion of mRNA into cDNA by reverse transcription.

True Single Molecule Sequencing (tSMS™) andlor Direct RNA. Sequencing (DRS™) are useful techniques for quantifying gene expression that can be readily adapted for detecting and quantifying the expression a CD99 gene. These sequencing-by-synthesis technologies can be performed on mRNAs derived from a tissue sample or cell without the need for prior reverse transcription or PCR amplification.

Direct RNA sequencing technology (Helicos BioSciences Corporation, Cambridge, MA) and transcriptome profiling using single-molecule direct RNA sequencing are described in Ozsolalc et al., Nature 461(7265):814-818 (2009) and Ozsolak and Milos, Methods Mot Blot 733:51-61 (2011). True Single Molecule and Direct RNA Sequencing technologies are further described in U.S. Patent Publication Nos. 2008/0081330, 2009/0163366, 2008/0213770, 2010/0184045, 2010/0173363, 2010/0227321, 2008/0213770, and 2008/0103058 as well as U.S. Patent Nos. 7,666,593; 7,767,400; 7,501,245; and 7,593,109, each of which is hereby incorporated by reference in its entirety.

mRNAs encoded by a CD99 gene can be directly sequenced by True Single Molecule and Direct RNA Sequencing technologies by utilizing specific sequencing primers that are designed based upon the CD99 inRNA sequences (e.g., as presented in Table 2A or which are otherwise known and readily available to those skilled in the art).

Methods for Inhibiting the Growth and/or Survival of a CD99⁺ Cell Associated with AML and/or MDS and for Treating a Patient Afflicted with an AML and or an MDS Exhibiting Elevated CD99⁺ Expression

The present disclosure further provides therapies that involve administering a composition comprising one or more anti-CD99 antibodies to a human patient for treating an AML and/or an MDS wherein the AML and/or an MDS exhibits elevated expression of CD99.

The amount of the anti.-CD99 antibody that will be effective in the treatment of AML and/or an MDS characterized by elevated CD99 expression can be determined by standard clinical techniques. In vitro assays may optionally be employed to help identify optimal dosage ranges. The precise dose to he employed in the formulation will also depend on the route of administration, and the seriousness of the disease or disorder. Effective doses may be extrapolated from dose-response curves derived from in vitro or animal model test systems.

The compounds or pharmaceutical compositions of the invention can be tested in vitro, and then in vivo for the desired therapeutic or prophylactic activity, prior to use in humans. For example, in vitro assays to demonstrate the therapeutic or prophylactic utility of a compound or pharmaceutical composition include the effect of a compound on a cell line or a patient tissue sample. The effect of the compound or composition on the cell line and/or tissue sample can be determined utilizing techniques known to those of skill in the art including, but not limited to proliferation and apoptosis assays. In accordance with the present disclosure, in vitro assays that can be used to determine whether administration of a specific compound is indicated, include in vitro cell culture assays in which a patient tissue sample is grown in culture, and exposed to or otherwise administered a compound, and the effect of such compound upon the tissue sample is observed.

The present disclosure provides methods of treatment and inhibition by administration to a subject of an effective amount of an anti-CD99 antibody or pharmaceutical composition as described herein. In one aspect, the compound is substantially purified such that the compound is substantially free from substances that limit its effect or produce undesired side-effects.

Methods of treatment and inhibition that employ one or more anti-.CD99 antibody may further comprise the administration of one or more additional compound such as, for example, a chemotherapeutic compound for the treatment of AML and/or MDS. Induction chemotherapy for AML can also include cytarabine (ara-C) and an anthracycline, such as daunorubicin. Cytarabine can be given as a continuous IV infusion for seven consecutive days while the anthracycline is generally given for three consecutive days as an IV push. The AML subtype acute promyelocytic leukemia is treated with all-trans-retinoic acid (ARA), often combined with an anthracycline and/or Arsenic Trioxide.

In the case of patients at high risk of relapse (e.g. those with high-risk cytogenetics, antecedent MDS, or therapy-related MDS/AML), allogeneic stem cell transplantation is usually recommended if the patient is able to tolerate a transplant and has a suitable donor. For good-prognosis leukemias (i.e., inv(16), t(8;21), and t(15;17)), patients will typically undergo an additional three to four courses of intensive chemotherapy, known as consolidation chemotherapy. For patients with relapsed AML, anti-CD99 antibodies may be administered in combination with a hetnatopoietic stem cell transplant, either as pre-transplant cytoreduction or post-transplant prophylaxis.

Various delivery systems are known and can be used to administer a composition of the present disclosure, fur example, encapsulation in liposomes, micropartieles, microcapsules, receptor-mediated endocytosis (see, e.g., Wu and Wu, J. Biol. Chem. 262:4429-4432 (1987)), and the like as will be known by one of skill in the art.

Methods of administration include, but are not limited to, iritraderinal, intramuscular, intraperitoneal, intravenous, subcutaneous, intranasal, epidural, and oral routes. The anti-CD99 antibodies or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the anti-CD99 antibodies or compositions into the central nervous system by any suitable route, including intraventricular and intrathecal injection. Intraventricular injection may be facilitated by an intraventricular catheter, for example, attached to a reservoir, such as an Ommaya reservoir. Pulmonary administration can also be employed, for example, by use of an inhaler or nebulizer, and formulation with an aerosolizing agent,

It may be desirable to administer the anti-CD99 antibodies or compositions of locally to the area in need of treatment; this may be achieved by, for example, local infusion during surgery, topical application, by injection, by means of a catheter, by means of a suppository, or by means of an implant, said implant being of a porous, non-porous, or gelatinous material, including membranes, such as sialastic membranes, or fibers.

The ai i-CD99 antibody can be delivered in a vesicle, such as a liposome (Langer, Science 249:1527-1533 (1990)) or in a controlled release system. A controlled release system can be placed in proximity of the therapeutic target, thus requiring only a fraction of the systemic dose (see, e.g., Goodson, in Medical Applications of Controlled Release, Vol. 2, pp. 115-138 (1984)).

Intravenous infusion of a compositions comprising an anti-CD99 antibody may be continuous for a duration of at least about one day, or at least about three days, or at least about seven days, or at least about 14 days, or at least about 21 days, or at least about 28 days, or at least about 42 days, or at least about 56 days, or at least about 84 days, or at least about 112 days.

Continuous intravenous infusion of a composition comprising an anti-CD99 antibody may be for a specified duration, followed by a rest period of another duration, For example, a continuous infusion duration may be from about 1 day, to about 7 days, to about 14 days, to about 21 days, to about 28 days, to about 42 days, to about 56 days, to about 84 days, or to about 112 days. The continuous infusion may then be followed by a rest period of from about 1 day, to about 2 days to about 3 days, to about 7 days, to about 14 days, or to about 28 days. Continuous infusion may then be repeated, as above, and followed by another rest period.

Regardless of the precise continuous infusion protocol adopted, it ill be understood that continuous infusion of a. composition comprising an anti-CD99 antibody will continue until either desired efficacy is achieved or an unacceptable level of toxicity becomes evident.

Within some aspects of the, present disclosure, methods for inhibiting the growth and/or survival of a CD99+ cell associated with AML and/or MDS and methods for treating a patient afflicted with an AML and/or an MDS exhibiting elevated CD99+ expression may further comprise the treating the cell with or administering to the patient a compound that promotes the mobilization of AML and/or MDS cells as well as associated hematopoietic and/or leukemic stern cells, respectively, front the bone marrow to the peripheral blood. Exemplary agents for achieving such mobilization have been described in the art and include G-CSF (Pabst et al., Blood 119(23):5367-5373 (2012)) and Plerixafor (Uy et al., Blood 119(17):3917-3924 (2012).

It will be understood that, unless indicated to the contrary, terms intended to be “open” (e.g., the term “including” should be interpreted as “including but not limited to,” the term “having” should be interpreted as “having at least,” the term “includes” should be interpreted as “includes but is not limited to,” etc.). Phrases such as “at least one,” and “one or more,” and terms such as “a” or “an” include both the singular and the plural.

It will be further understood that where features or aspects of the disclosure are described in terms of Markush groups, the disclosure is also intended to be described in terms of any individual member or subgroup of members of the Markush group. Similarly, all ranges disclosed herein also encompass all possible sub-ranges and combinations of sub ranges and that language such as “between,” “up to,” “at least,” “greater than,” “less than,” and the like include the number recited in the range and includes each individual member.

All references cited herein, whether supra or infra, including, but not limited to, patents, patent applications, and patent publications, whether U.S., PCT, or non-U.S. foreign, and all technical and/or scientific publications are hereby incorporated by reference in their entirety.

While various embodiments have been disclosed herein, other embodiments be apparent to those skilled in the art. The various embodiments disclosed herein are for purposes of illustration and are not intended to be limiting, with the true scope and spirit being indicated by the claims.

The present disclosure will be further described with reference to the following non-limiting examples. The teaching of all patents, patent applications and all other pUblications cited herein are incorporated by reference in their entirety

Antibodies used in the following experiments are all commercially available, e.g., from Abeam, Cambridge Mass.

### EXAMPLES

### Example 1

Anti-CD99 12E7 Antibody is Cytotoxic to and Induces Apoptosis in CD99+ MDS Cell MDS Primary Cells, AML Cell Lines, and Primary AML Blasts

This Example demonstrates that anti-CD99 antibody 12E7-mediated ligation of CD99 on CD99-expressing MDS cell lines, MDS primary cells. AML cell lines, and primary AML blasts is eytotoxic to and induces apoptosis in those cells. Moreover, the cytotoxicity due to ligation of CD99 occurs in the absence of antibody effector function and, therefore, 12E7-mediated cytotoxicity and apoptosis is independent of complement-dependent cytotoxicity (CD( )andlor antibody-dependent cell-mediated cytotoxicity (ADC( )

Ligation of CD99 on MDS cell line MDS92 (Tohyania Br. J, Haematol. 91:795 (1995)) with 20 of anti-CD99 antibody designated 12E7 (Levy et al., Proc. Natl, Acad Sci. USA, 76:6552 (1979)) is cytotoxic to those MDS92 cells as evidenced by a 128-fold decrease in MDS92 cell number (p<0.001) at 72 hours as compared to MDS92 cell number in the presence of 2( )of an isotype control antibody. FIG. 1A. Analysis of AAD vs. Annexin V fluorescence values demonstrated effector-independent apoptosis (as evidenced by a 77% increase in annexin V positivity (p<0.001) of MDS92 cells following 72 hour ligation of CD99 with 10 μg/ml of12E7 as compared to the absence of apoptosis of MDS92 cells following 72 hour in the presence of 20 μg/ml of an isotype control antibody. FIG. 1B.

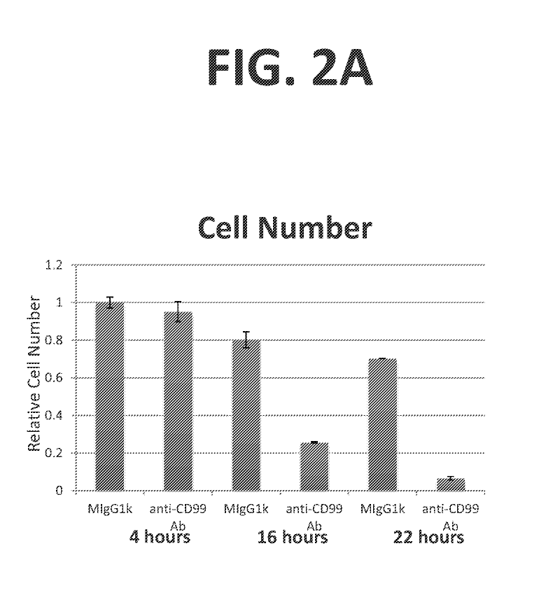

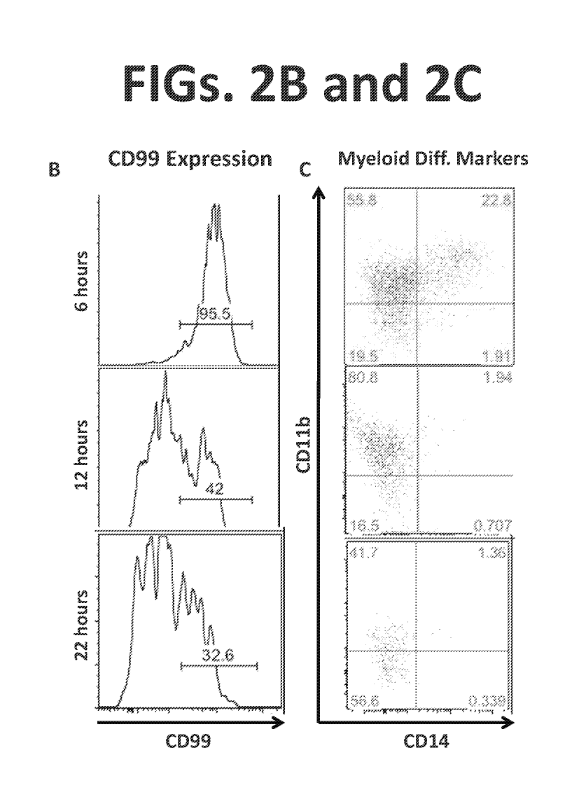

12E7-mediated cytotoxicity to CD99-F MDS92 cells was further supported by the time-dependent 128-fold decrease in MDS92 cell number at 22 hours (p<0.001) following ligation of CD99 with anti-CD99 antibody 12E7 as compared to MDS92 cell number in the presence of an isotype control antibody. FIG. 2A. CD99 cell-surface expression on MDS92 cells exhibited a time-dependent decrease following ligation of CD99 with anti-CD99 antibody 12E7. FIG. 2B. Cell-surface expression of myeloid differentiation markers CD11b and CD14 exhibited a time-dependent decrease following ligation of CD99 with anti-CD99 antibody 12E7. FIG. 2C.

Ligation of CD99 on primary CD34⁺ MDS cells with the 12E7 anti-CD99 antibody is cytotoxic to those primary MDS cells as evidenced by a 10-fold decrease in cell number relative to no antibody control and an 8-fold decrease in cell number relative to isotype (IgG) control antibody after 48 hours. FIG. 3.

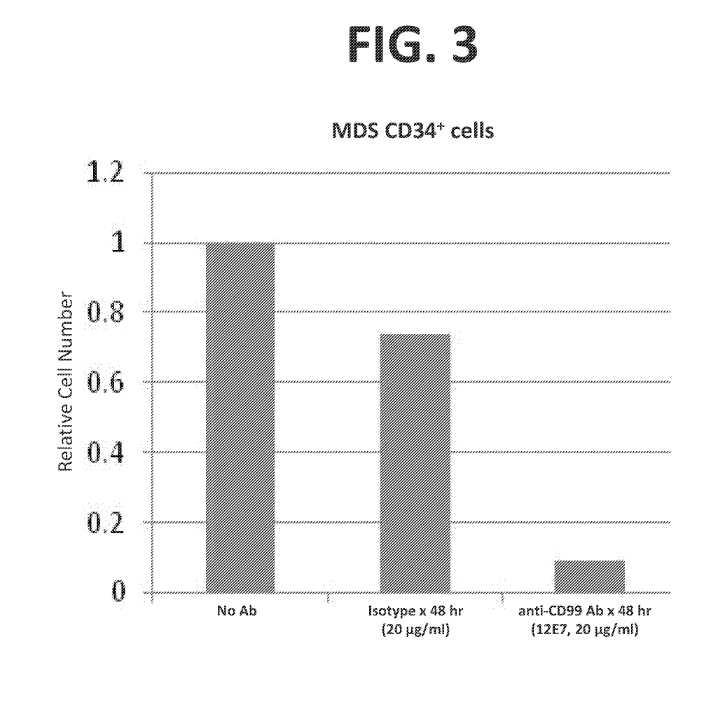

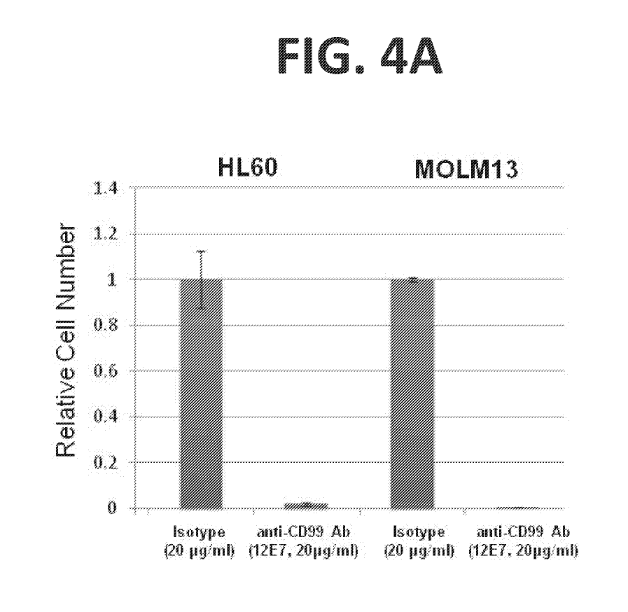

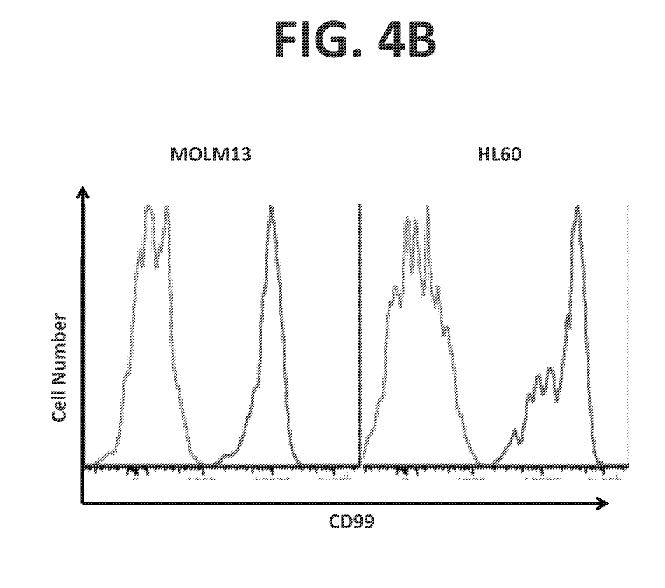

Ligation of CD99 on AML cell lines expressing high levels of CD99, such as HL60 and MOLM13 with the 12E7 anti-CD99 antibody is cytotoxic to those AML cell lines /as evidenced by a 49-fold decrease (HL69; p<0.001) and a 70-fold decrease (MOLM13; p<0.001) in cell number relative to isotype control antibody after 72 hours. FIG. 4A. CD99 is expressed at high levels on MOLM13 and HL60 cells compared with isotype control.

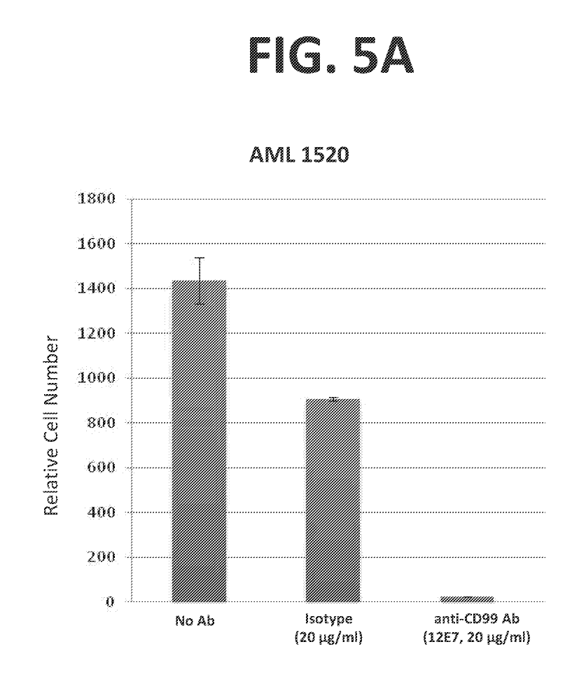

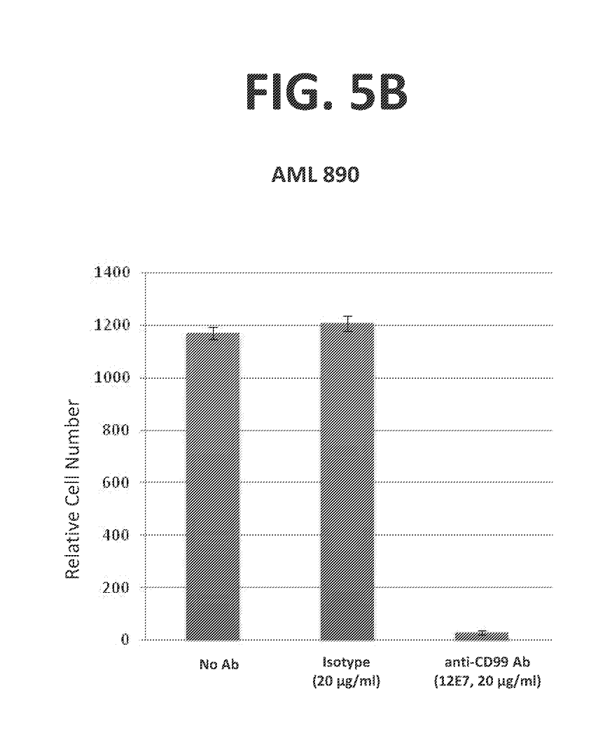

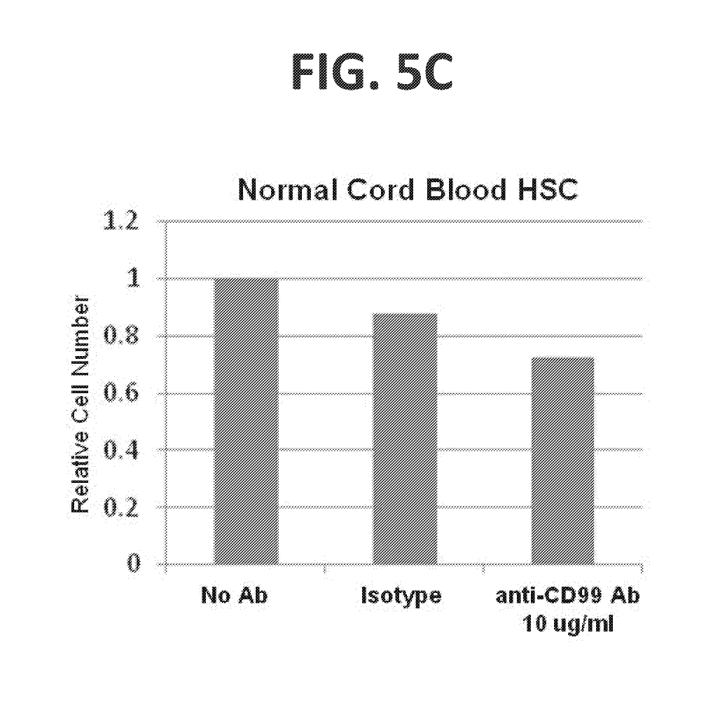

Ligation of CD99 on CD99-expressing primary AML 1520 blast cells with 12E7 anti-CD99 antibody is cytotoxic to those primary AML blast cells as evidenced by a 57-fold decrease (p<0.001) in cell number after 48 hours. FIG. 5A. Ligation of CD99 on CD99-expressing primary AML 890 blast cells with 12E7 anti-CD99 antibody is cytotoxic to those primary AML blast cells as evidenced by a 48-fold decrease (p<0,001) in cell number after 48 hours. FIG. 5B. 12E7 anti-CD99 antibody has only a modest cytotoxic effect on normal cord blood HSC as evidenced by a 1.4-fold decrease in HSC cell number after 80 hours. FIG. 5C. This indicates that anti-CD99 have a substantial therapeutic window.

### Example 2

Comparative Transcriptome Analysis of Highly Purified Lineage Negative [Lin−] CD38+CD34−CD.90+CD45RA-Hematopoietic Stem Cells

This Example demonstrates that CD99 is expressed on disease-initiati stem cells in MDS and AML. In AML, the LSC has been demonstrated to exhibit self-renewal and the ability to differentiate into non-self renewing progeny that comprise the bulk of disease cells (Lapidot et al., Nature 367:645-648 (1994) and Bonnet and Dick, Nat. Med. 3:730-737 (1997)), representing the first malignancy to fulfill criteria laid forth by the cancer stem cell hypothesis. Therefore targeting LSCs with anti-CD99 antibodies promises to attack disease-initiating stem cells in AML.

It has been demonstrated that MDS is initiated in HSCs, showing that MDS HSCs contain disease associated cytogenetic abnormalities and can engraft disease in immunodeficient animals. Nilsson et al., Blood 100:259-267 (2002); Nilsson et al., Blood 110:3005-3014 (2007); Tehranchi et al., New Engl. J. Med. 363:1025-1037 (2010); Nilsson et al., Blood 96:2012-2021 (2000); Pang et al., Proc. Natl. Acad. Sci. 110:3011-3016 (2013); and Will et al., Blood 120:2076-2086 (2012). Thus, targeting MDS HSCs with anti-CD99 antibodies promises to attack disease-initiating stem cells in MDS.

Many investigators have identified cell surface proteins preferentially expressed on AML LSCs compared to normal HSCs (Jin et al., Nat. Med. 12:1167-1174 (2006); Majeti et al., Cell 138:286-299 (2009); Jan et al., Proc. Nati. Acad. Sci. 108:5009-5014 (2011); Jin et al., Cell Stem Cell 5:31-42 (2009); van Rhenen et al. Blood 110:2659-2666 (2007); Saito et al., Sci. Transl. Med. 2:17ra19 (2010); these represent promising targets for therapies that may selectively target AML LSCs while sparing HSCs. To date, cell surface protein expression in MDS HSCs has not been carefully characterized, and no stem cell targeted therapies have been evaluated in this disease.

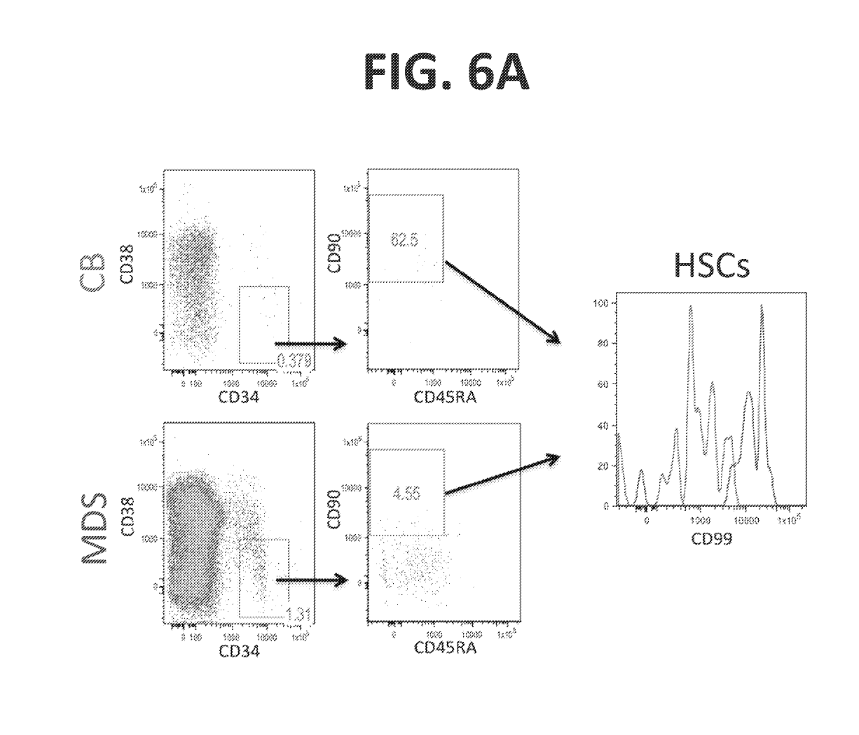

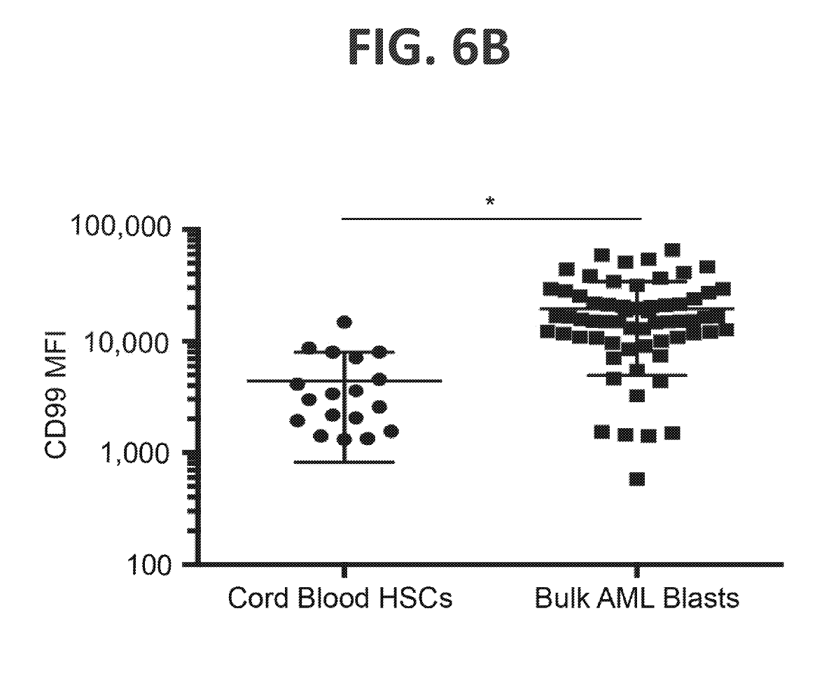

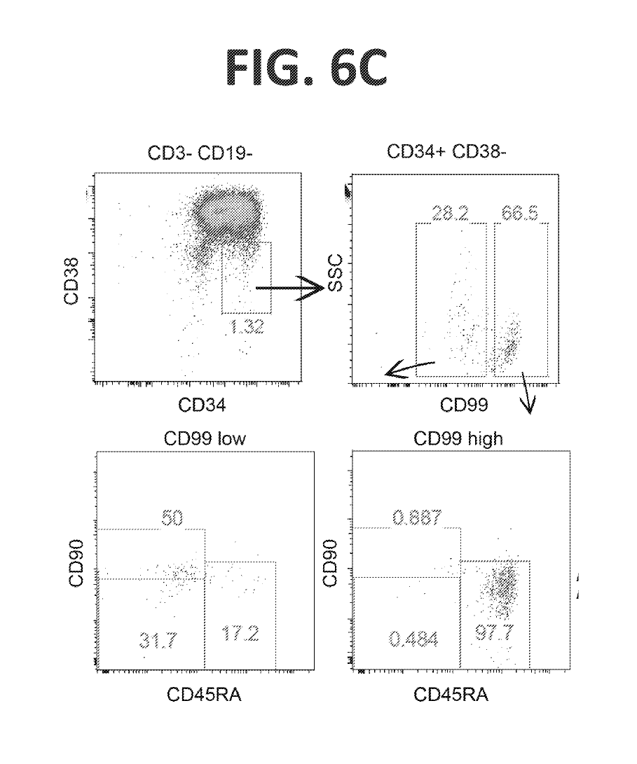

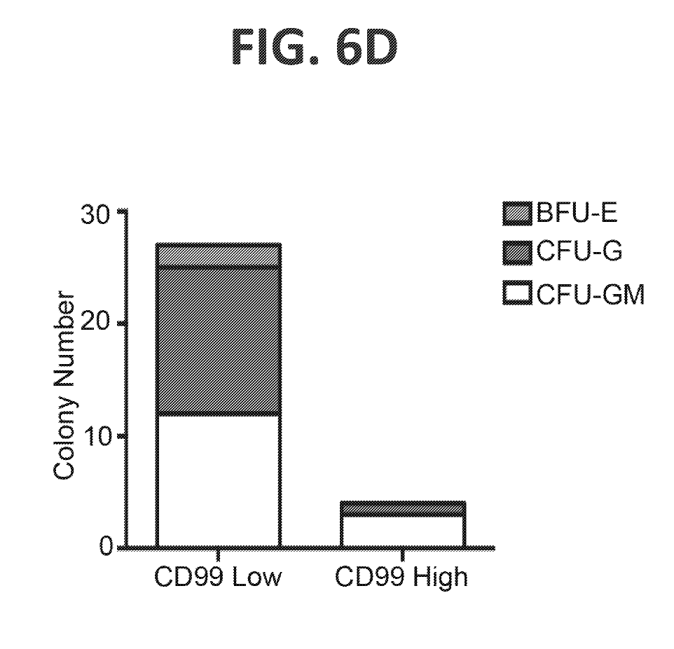

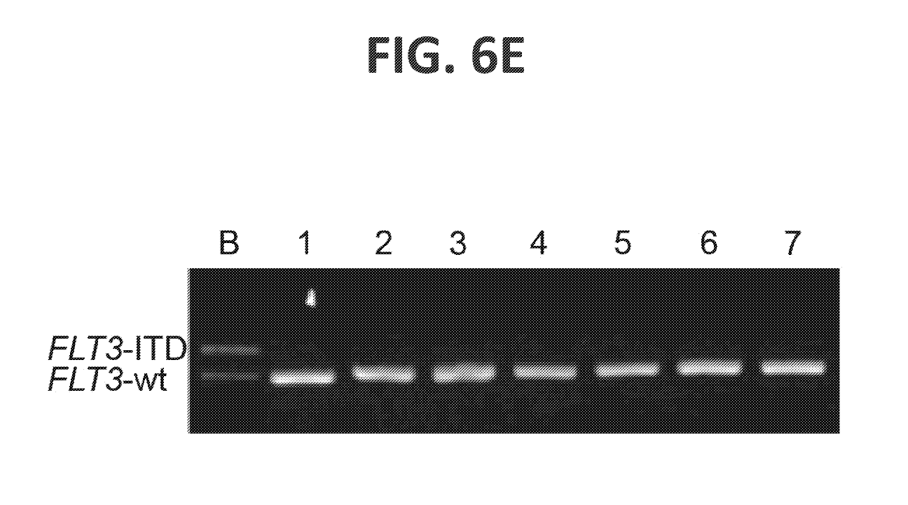

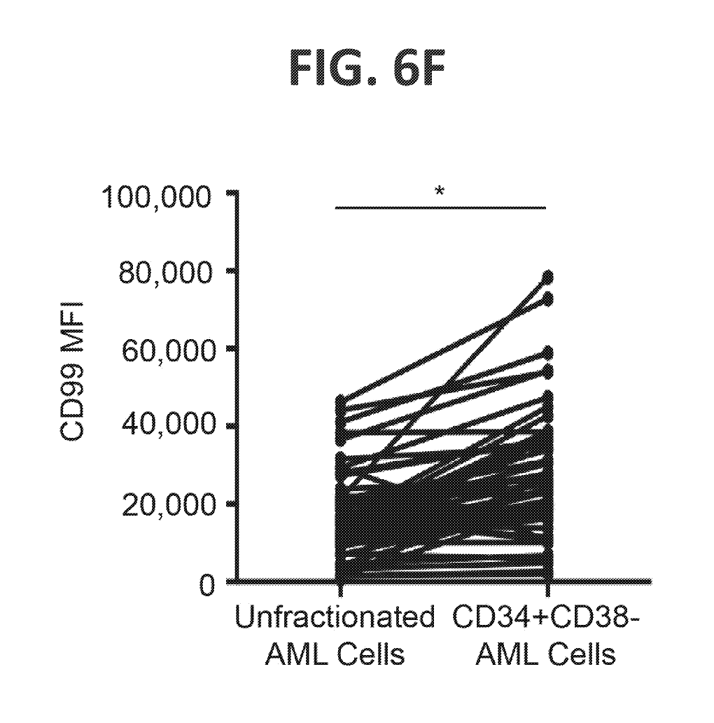

A comparative transcriptome analysis of highly purified HSCs (lineage negative [Lin−] CD38+CD34−CD90+CD45RA−) was performed from eight patients with a myelodysplastic syndrome (MDS), seven low-risk and one intermediate-risk (Greenberg et al. Blood 89:2079-2088 (1997))) and eleven age-matched normal controls (McGowan et al., Blood 118:3622-3633 (2011)), identifying 25 dysregulated cell surface protein transcripts (FDR-(0.1), To identify cell surface markers specific for MDS HSCs, flow cytometry (FC) was used to confirm cell surface expression of these markers as compared with cord blood (CB) HSC controls in validation cohorts of 26 therapy-related MDS specimens and 37 de novo MDS specimens, identifying CD99 as the most frequently overexpressed (85% and 73% of cases, respectively, FIG. 6A). The high frequency of CD99 expression across these randomly selected MDS cases suggests that anti-CD99 antibodies will be effective against a broad range of MDS subtypes.

To explore the relevance of CD99 as a marker of other myeloid malignancies, the expression of CD99 was evaluated by F(on leukemic blasts ftom 78 paired diagnosis and relapse AML samples and Observed elevated levels of CD99 expression on 81% of diagnostic samples and 83% of relapse samples (average 9.2-fold increase, p<0.0001, FIG. 6B), demonstrating that CD99 is overexpressed in AML as well as MDS. CD99 expression was stable between diagnosis and relapse, suggesting that anti-CD99 antibodies may be effective at many disease states (both at initial diagnosis and at relapse). in one experiment, CD99 surface expression was significantly increased (p=0.047) at relapse compared to diagnosis.

To examine whether expression of CD99 can distinguish leukemic cells from normal hematopoietic stem and progenitor cells (HSPCs), CD99 cell surface expression was evaluated in the stem-cell enriched CD34+CD38− fraction of AML. Within this fraction, “CD99 high” cells resembled lymphoid-primed multipotent progenitors (LNIPPs, CD34+CD38−CD90−CD45RA+), consistent with the immunophenotype adopted by most human AMLs (Goardon et al., Cancer Cell 19:138-152 (2011)), while “CD99 low” cells resembled the HSCs/multipotent progenitors (MPPs) that predominate in normal hematopoiesis (FIG. 6C). Majeti et al., Cell Stem Cell 1:635-645 (2007). When “CD99 low” cells were FACS-purified into methylcellulose cultures, they demonstrated the robust myeloid colony formation characteristic of normal IISCs, but not observed with LSCs (FIG. 60). These colonies were harvested and demonstrated by allele specific PCR that they lacked molecular markers (e.g., FL T3-ITD) associated with the corresponding AML, consistent with their derivation from residual normal or pre-leukemic IISCs (FIG. 6E). Jan et al., Sci. Trans. Med. 4:149ra118 (2012). This demonstrates the specificity of CD99 expression for leukemic stem cells (LSCs) as compared to residual normal hematopoietic stem cells (USCs) within the same patient, suggesting that anti-CD99 antibodies will have a wide therapeutic window.

The CD34⁺CD38 fraction of AML has been shown to be enriched for LSCs (Bonnet and Dick, Nat. Med. 3:730-737 (1997) and Goardon etal., Cancer Cell 19:138-152 (2011)) and it was found that this fraction had consistently higher levels of CD99 expression in AML compared with more differentiated AML blasts (FIG. 6F). To test whether CD99 expression enriches for functional ILSCs, the top and bottom 10% of CD99 expressors was transplanted within the LSC enriched CD34⁺ CD38⁻CD90⁻CD45RA⁻ fraction of a primary AML into sublethally irradiated (185 cGy) NSG mice at limiting dilution, finding that leukemia-initiating capacity (LIC) was restricted to CD99 high cells (1 in 24,401 cells for the top 10%, no engraftment from t ie bottom 10% (See, Table 3A). The top 10% and bottom 10% of CD99 expressors were purified from the “LMIT-like” LSC enriched fraction of the AML specimen UI'enn 2741 and transplanted at limiting dilution into sublethaily irradiated NSG mice (185 cGy). Leukemic engraftment (using a cut-off >0.1% human CD45+ cells) was only observed in mice transplanted with “top 10%” cells. This validates CD99 expression as a specific marker of functionally relevant leukemia initiating cells, suggesting that anti-CD99 antibodies will preferentially kill this disease-initiating cell population.

TABLE 3A
Leukemia-initiating capacity (LIC) is restricted to CD99 high cells

| | | | | | Leukemia |
|---|---|---|---|---|---|
| | | | | | Initiating |
| | CELL DOSE TRANSPLANTED | | | | Cell |
| Fraction | 360,000 | 30,000 | 3,000 | 300 | Frequency |
| Top 10% | 4/4 | 3/4 | 0/2 | 0/4 | 1 in 24,401 |
| CD99 | | | | | |
| Expressing | | | | | |
| LSCs | | | | | |
| Bottom 10% | 0/4 | 0/3 | 0/3 | 0/4 | Not |
| CD99 | | | | | Evaluable |
| Expressing | | | | | |
| LSCs | | | | | |

A similar dilution analysis experiment was performed with another independent AML sample UPenn 2522, Table 3B shows limiting dilution analysis of ⁻UPenn 2522 transplantation of CD19−CD3−CD45(dim) SSC(low) leukemic blasts with high or low CD99 expression. In Table 3B, the top and bottom 15% of CD99 expressing “LMPP-like” LSC-enriched blasts from AML specimen UPenn 2522 were similarly purified and transplanted at limiting dilution into NSG mice. Leukemic engraftment was only observed in mice transplanted with “top 15%” CD99 expressing blasts.

TABLE 3B
Limiting dilution analysis of UPenn 2522
transplantation of CD19−CD3− CD45(dim)
SSC(low) leukemic blasts with high or low CD99 expression

| | CELL DOSE TRANSPLANTED | | | | LSC |
|---|---|---|---|---|---|
| Fraction | 380,000 | 38,000 | 3,800 | 380 | Frequency |
| Top 15% of | 4/4 | 1/4 | 0/4 | 0/4 | 1 in 105,688 |
| CD99 | | | | | (95% CI 1 in |
| Expressing | | | | | 58,204 to 1 |
| Blasts | | | | | in 191,911) |
| Bottom 15% | 0/4 | 0/4 | 0/4 | 0/4 | Not |
| of CD99 | | | | | Evaluable |
| Expressing | | | | | |
| Blasts | | | | | |

Thus, it appears that CD99 is not only highly expressed in AML, but that it is enriched in functional LSCs. This represents the first LSC marker reported to have this feature. Other LSC markers were found to be expressed at roughly equal levels on LSCs and more differentiated AML blasts (e.g., CD44, CD47, and TIM3; data not shown). Jin et al., Nat. Med. 12:1167-1174 (2006); Majeti et al., Cell 138:286-299 (2009); and Jan et al., Proc. Natl. Acad. Sci. U.S.A. 108:5009-5014 (2011).

To further investigate, heterogenous expression of CD99 among patient AML blasts was evaluated. Patient AML blasts were fractionated. Unfractionated cells were compared to fractionated CD34+CD38− AML cells and evaluated for expression level of selected possible LSC markers including CD99, CD44, CD123, CD47, and TIM3. Of several AML markers tested, only CD99 was found to be significantly enriched in fractionated leukemic stem cells (CD34+, CD38−) in a statistically relevant fashion among patient AML blasts. No such statistically relevant increased expression was seen for CD14, CD123, CD47, or TIM3.

The distribution of CD99 is much higher on leukemia cells than it is in normal HSPC's. Based on this observation, prospective separation of residual normal HSCs from leukemic cells was performed. An AML patient sample (MSK AML 003) was obtained. CD99 expression was evaluated by FC in CD3−CD19−CD34−CD38− cells from AML specimen MSK AML-003. The cell populations were FACS-sorted to >95% purity into “CD99 Low” and “CD99 High” groups. Both groups were plated in methylcellulose supplemented with cytokines (750 cells in triplicate). Normal myeloidlerythroid colonies formed only from the “CD99 low” fraction with the expected distribution of different types of hematopoietic cells (CFU-E, GEMM, CTU-M and CFU-G). In addition, the “CD99 low” derived colonies lacked the homozygous FLT3-ITD abnormality present in MSK AML-003.

In a separate experiment, the fractions from AML sample (MSK-003) was sorted into “CD99 high” (Lin-CD34+CD38−CD99+) and “CD99 low” (Lin−CD34−FCD38−CD99−) fractions which were xenotransptanted into NSG mice. Engraftment was assessed after 12 weeks. “CD99 low” cells demonstrated normal lympho-myeloid engraftment, consistent with the activity of residual normal hematopoietic stem cells (HSCs), as shown in Table 3C. In contrast, “CD99 high” cells formed leukemic engraftment in 4 of 4 mice at the high cell dose, as shown in Table 3C.

TABLE 3C
Engraftment after 12 weeks following xenotransplantion to NSG mice of
CD99 High and CD99 Low Cells

| | | | | Lympho- |
|---|---|---|---|---|
| | | Cell Dose | Leukemic | Myeloid HSC |
| AML Sample | Cell Population | Transplanted | Engraftment | Engraftment |
| MSK-003 | Lin-CD34+CD38−CD99+ | 75,000 | 4/4 | 0/4 |
| | Lin-CD34+CD38−CD99+ | 2,500 | 0/2 | 0/2 |
| | Lin-CD34+CD38−CD99− | 2,500 | 0/2 | 2/2 |

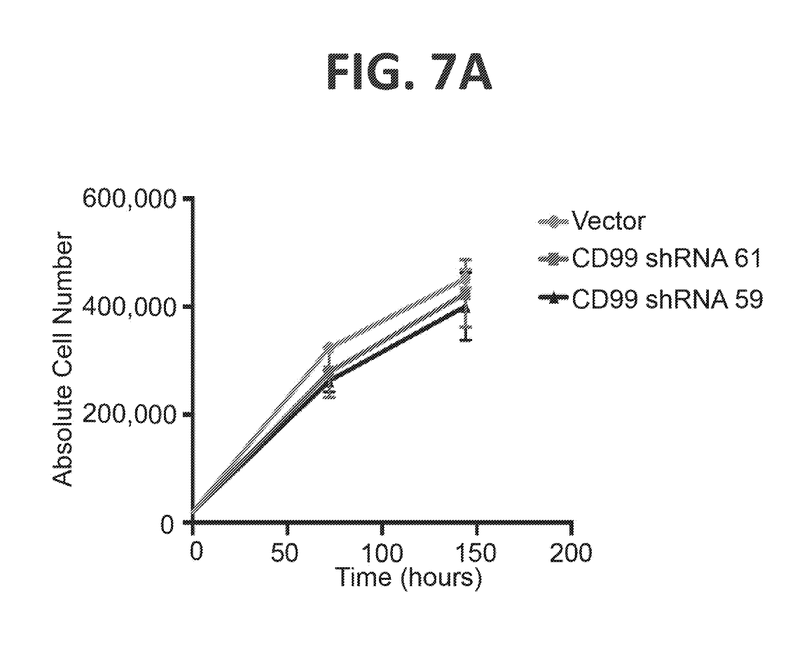

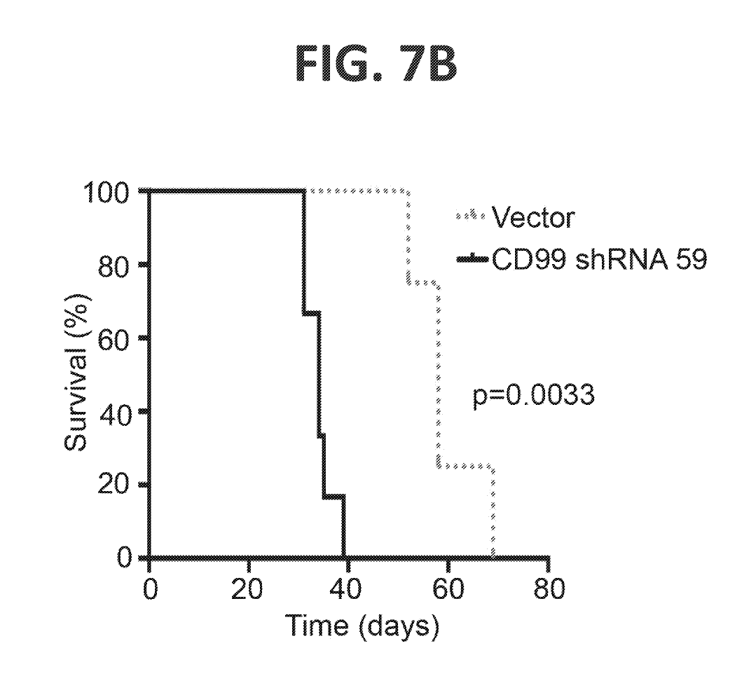

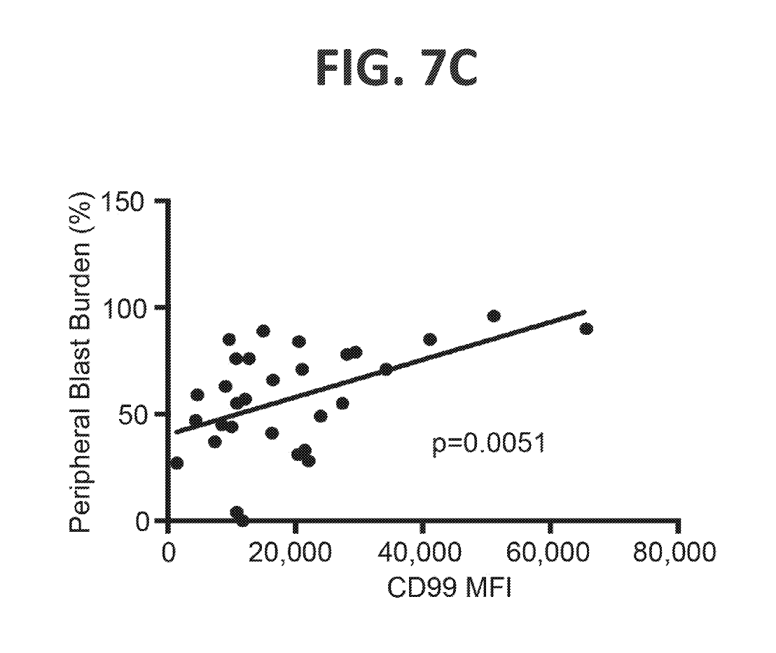

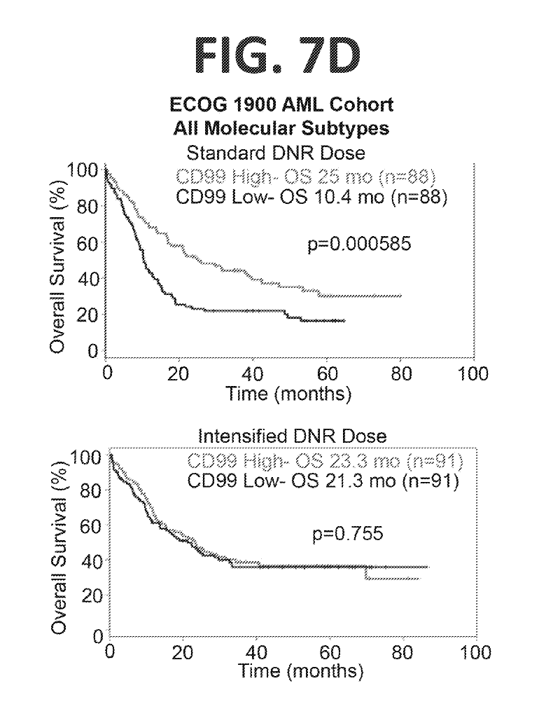

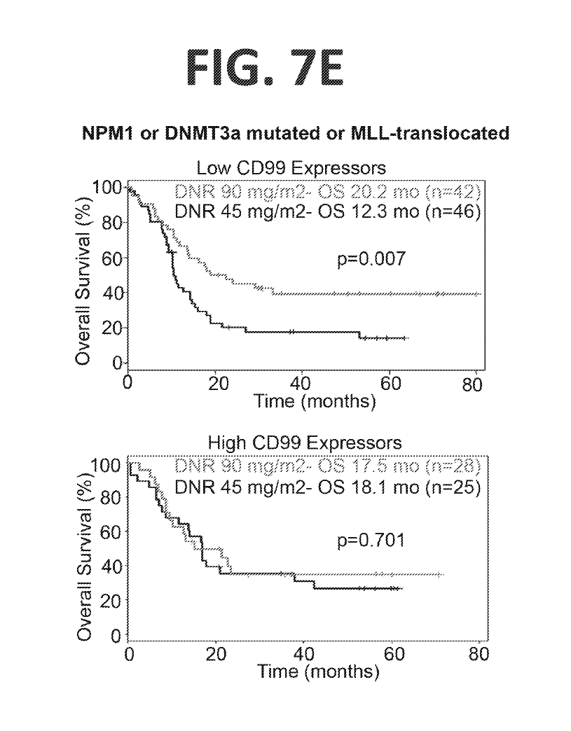

To characterize the function of CD99 in AML, MOL,M13 AML cells were stably transduced with a shRNA targeting CD99 (8.0-fold knockdown). Knockdown of CD99 in FIL60 cells with two shRNAs (2.3-fold and 8.3-fold with #61 and #59, respectively) did not significantly alter proliferation kinetics in vitro. Error bars represent±SD. Although this led to no significant difference in growth in vitro (FIG. 7A), sublethally irradiated NSG mice transplanted with these cells had significantly improved survival compared to vector controls (58d vs. 34d, p=0.02, FIG. 7B), suggesting that CD99 may foster more aggressive disease by modulating interaction with the microenvironment. Accordingly, high CD99 surface protein expression in human AML samples was significantly associated with an increase in BM disease burden (FIG. 7C). High CD99 expression is associated with more aggressive growth of AML in both mice and humans.

Gene expression signatures enriched in LSCs have been predictive of worse clinical outcomes (Majeti, Proc. Natl. Acad. Sci. U.S.A. 106:3396-3401. (2009) and Eppert, et al. Nature Medicine 17:1086-1093 (2011)), and overexpression of LSC markers such as CD 123 and CD47 correlate with increased leukemic burden and worse OS, respectively. Thus, it was hypothesized that CD99 mRNA expression in AML would enrich for “sternness” and predict for a worse prognosis. Transcriptome data available from 358 AML patients enrolled in an Eastern Cooperative Oncology Group (ECOG) clinical trial (E1900) was analyzed by comparing two doses of Daunorubicin (DNR) for initial disease treatment. Surprisingly, low CD99 expression correlated with a worse prognosis, and this appeared to be mitigated by receipt of the higher dose of DNR (FIG. 7D).

Moreover, within molecular subtypes of AML predicted to benefit from DNR intensification (NPM1 mutated, DNMT3a mutated, or MLL-rearranged; Patel et al, New Engl. J. Med. 366:1079-1089 (2012)), the benefit was limited to low CD99 expressors (FIG. 7E). Given the established role of CD99 in the transendothelial migration of monocytes and neutrophils in response to inflammation (Schenkel et al., Nat. Immunoi, 3:143-150 (2002) and Lou et al., J. Immunol. 178:1136-1143 (2007)), it was hypothesized that higher levels of CD99 mRNA expression lead to improved clinical outcomes in the context of chemotherapy by promoting mobilization of leukemic blasts through endothelium and into the PB, where they have been shown to be more susceptible to chemotherapy. Uy et a)., Blood 119:3917-3924 (2012),

### Example 3

CD99 Promotes Transendothelial Migration and Mobilization of Leukemic Blasts

To test the influence of CD99 expression on transendothelial migration of leukemic blasts, the AML cell line HL60 was stably transduced to overexpress CD99 (6.7-fold) and seeded it on human umbilical vein endothelial cells (HUVECs) grown to confluence on transwell membranes, allowing HL60s to migrate through the HUVECs towards the chemoattractant SIDE-1 (data not shown). Overexpression of CD99 led to a significant increase in the efficiency of transendothelial migration (3.72-fold increase, p<0.0009 at 4 hours, 2.93-fold at 28 hours. p=0.0065, FIG. SA). The efficiency of this migration could be variously enhanced or inhibited by the addition of different anti-CD99 mAbs (data not shown). Anti-CD99 antibodies that block transendothelial migration may be used in conjunction with mobilizing agents such as G-CSF or Plerixafor to “trap” mobilized leukemia cells in the peripheral blood, where the tumor cells may exhibit enhanced chemosensitivity as well as sensitivity to cytotoxic anti-CD99 antibodies, as described herein.

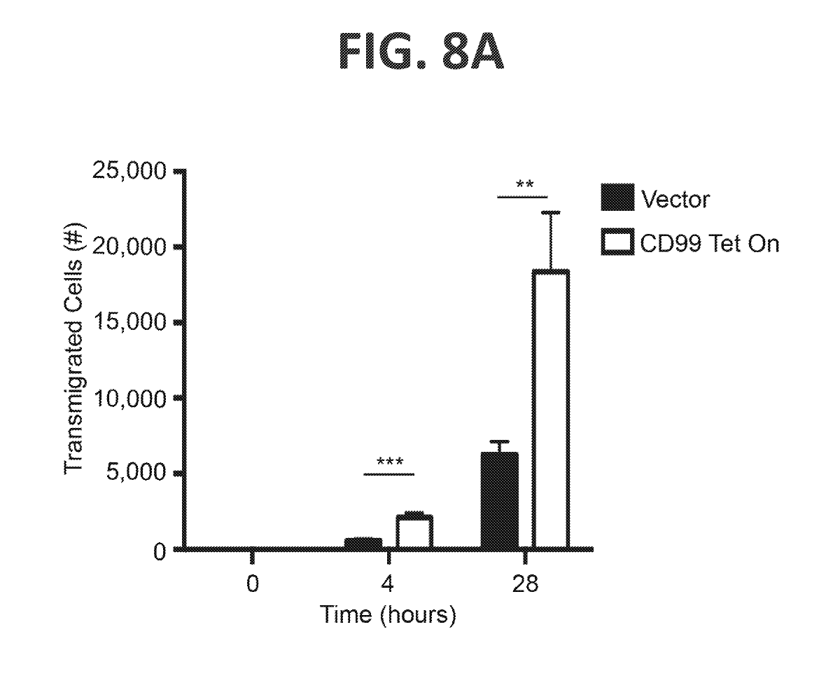

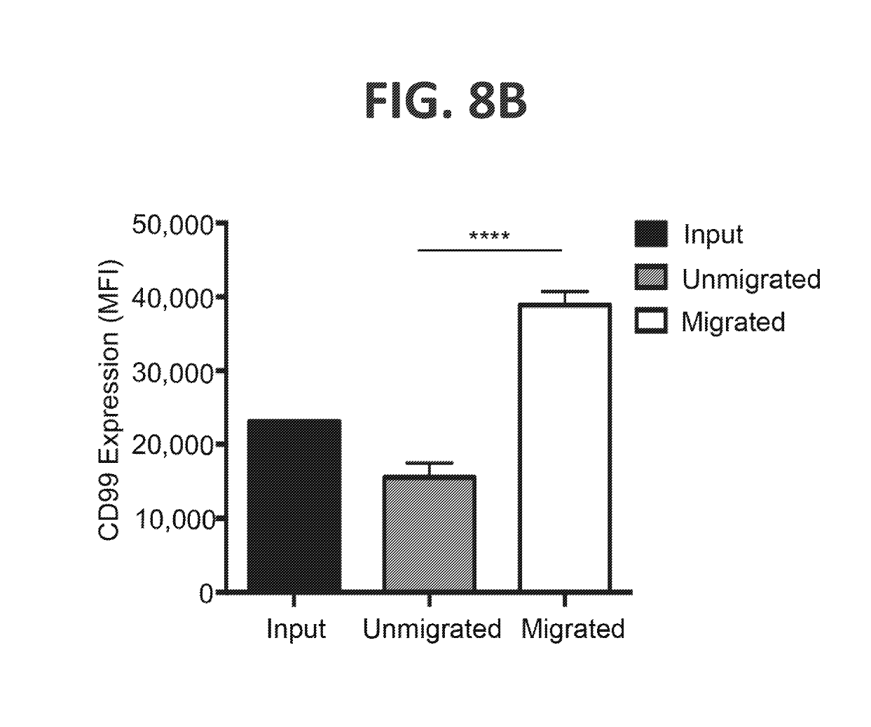

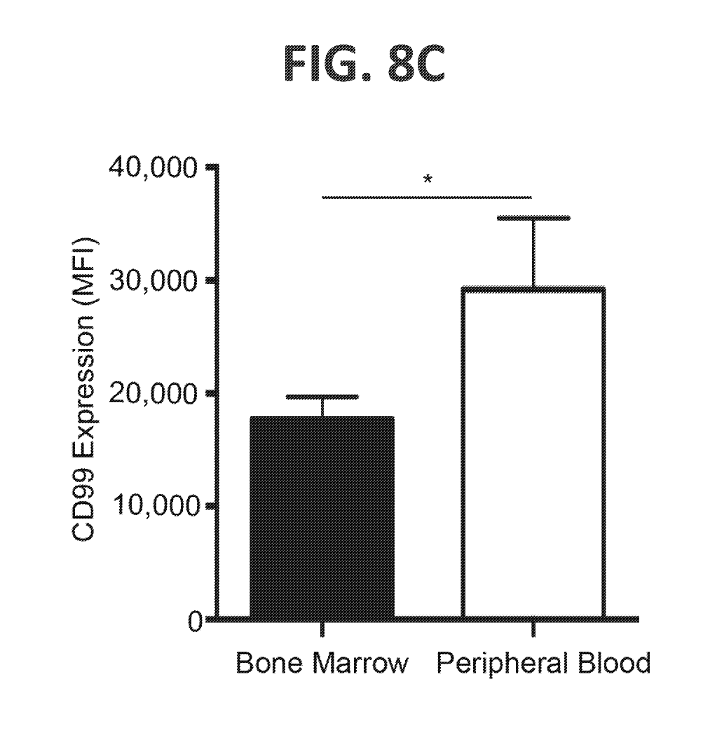

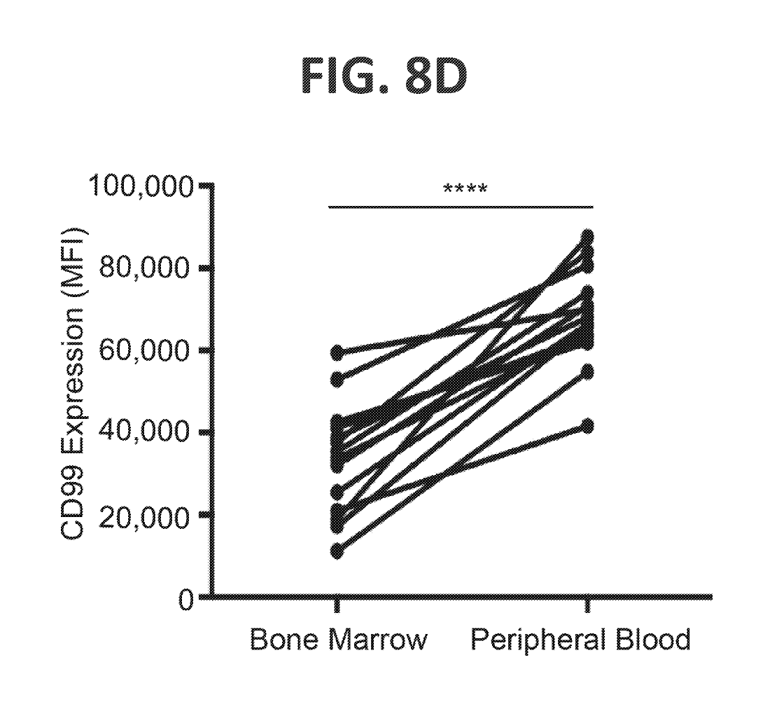

After 72-hrs. remaining unmigrated cells had significantly lower levels of CD99 expression as compared with migrated cells (3.65-fold lower, p=0.028, FIG. 8B). There was no correlation between CD99 expression and expression of the SDP-1 receptor CXCR4 (data not shown). CD99 surface expression was found to be significantly higher on AML specimens taken from the peripheral blood as compared with the BM (1.65-fold higher, p=0.028, FIG. 8C), and in a paired analysis of BM and PB taken simultaneously from primary AML xenografts, CD99 surface expression was significantly higher on leukemic blasts circulating in the PB as compared with the BM (2.32-fold increase, p<0.0001 , FIG. 8D). Finally, overexpression of CD99 in AML cell lines led to an increase in adhesion to tissue culture plates coated with stromal elements such as fibronectin and collagen (FIG. SE).

Together, these findings suggest that CD99 promotes mobilization of leukemic blasts by enhancing transendothelial migration and decreasing adhesion to stromal elements in the BM.

### Example 4

Anti-CD99 Monoclonal Antibodies (mAbs) are Directly Cytotoxic to AML and MDS Cells

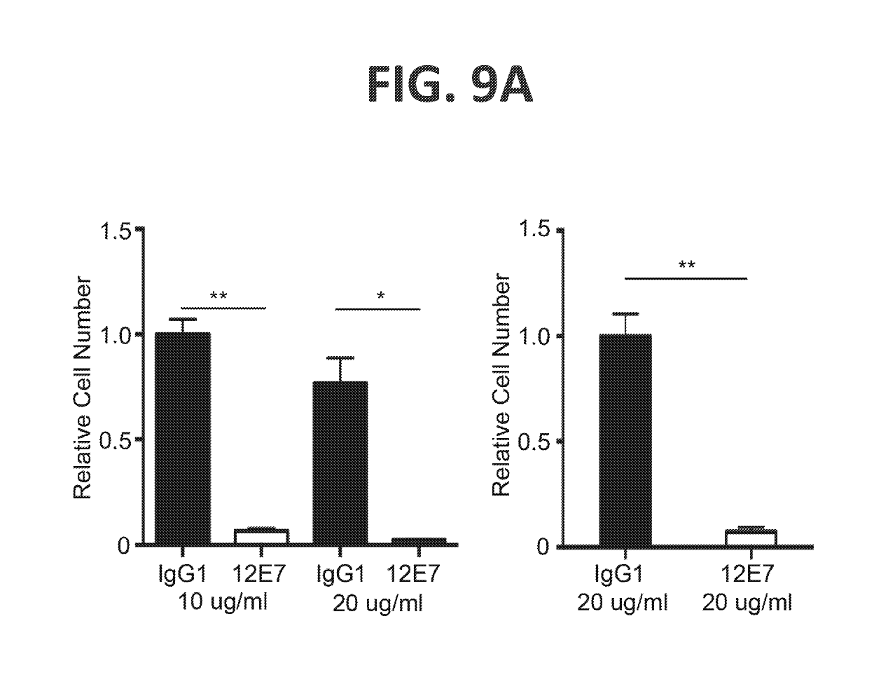

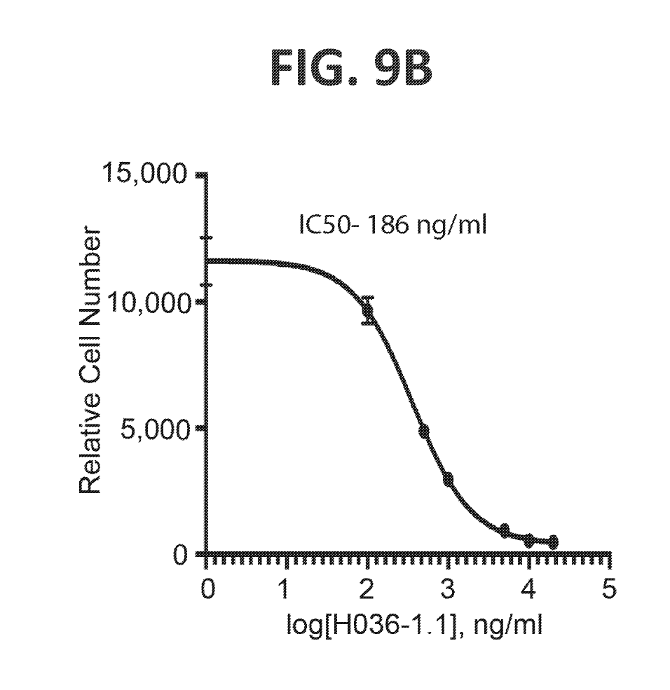

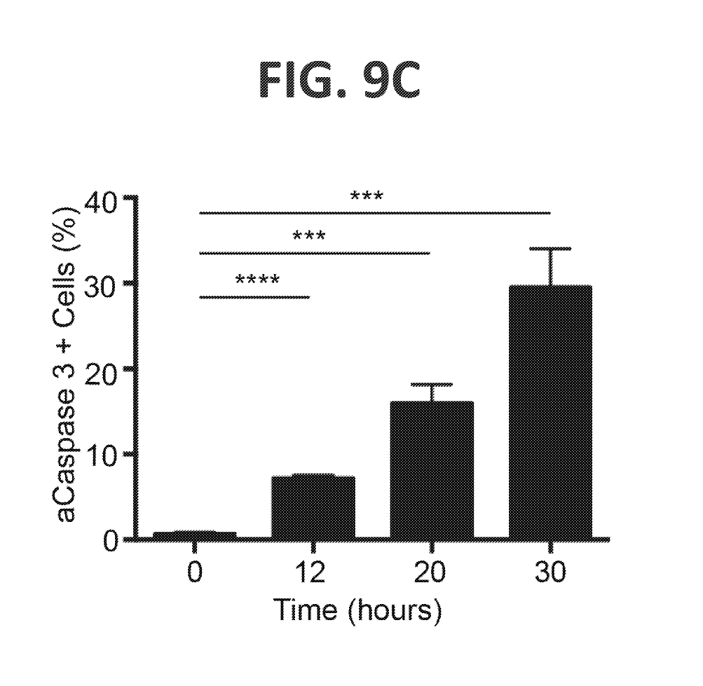

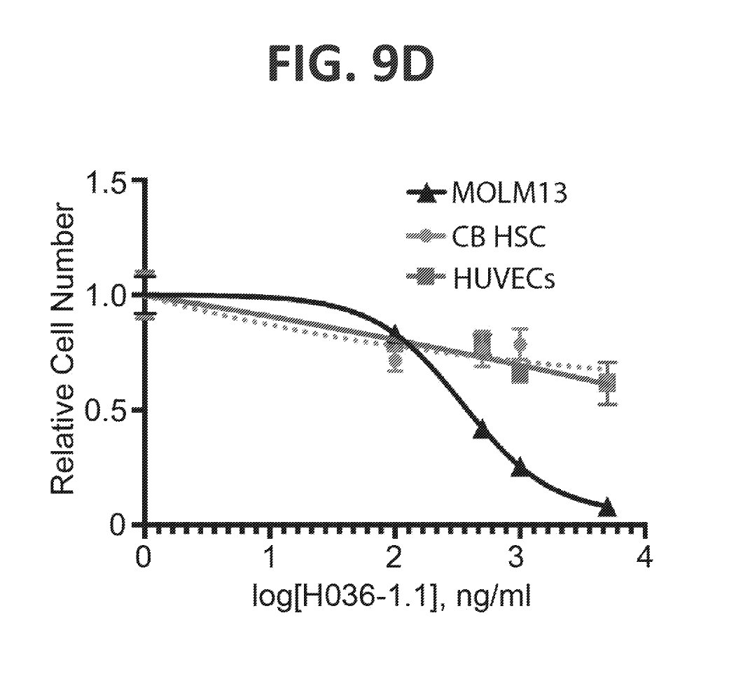

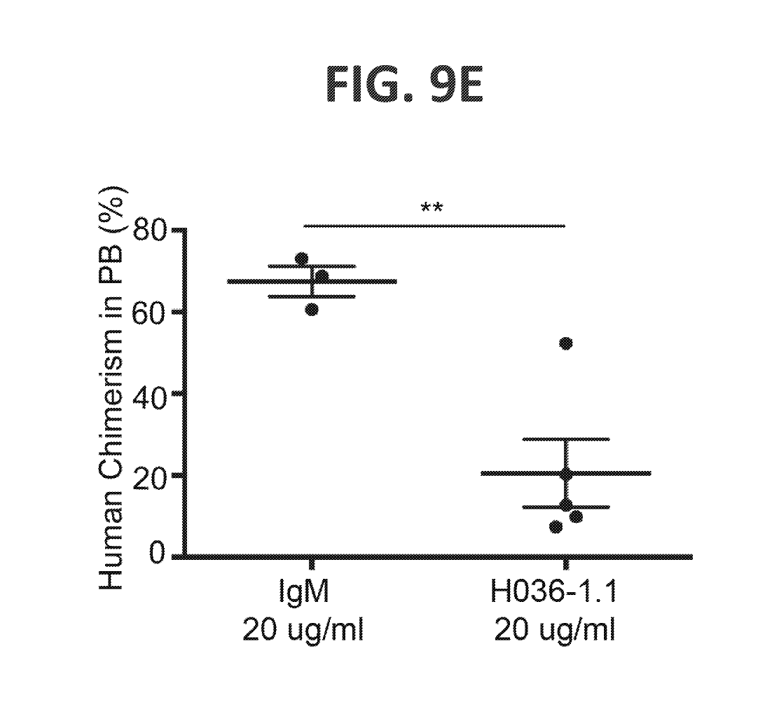

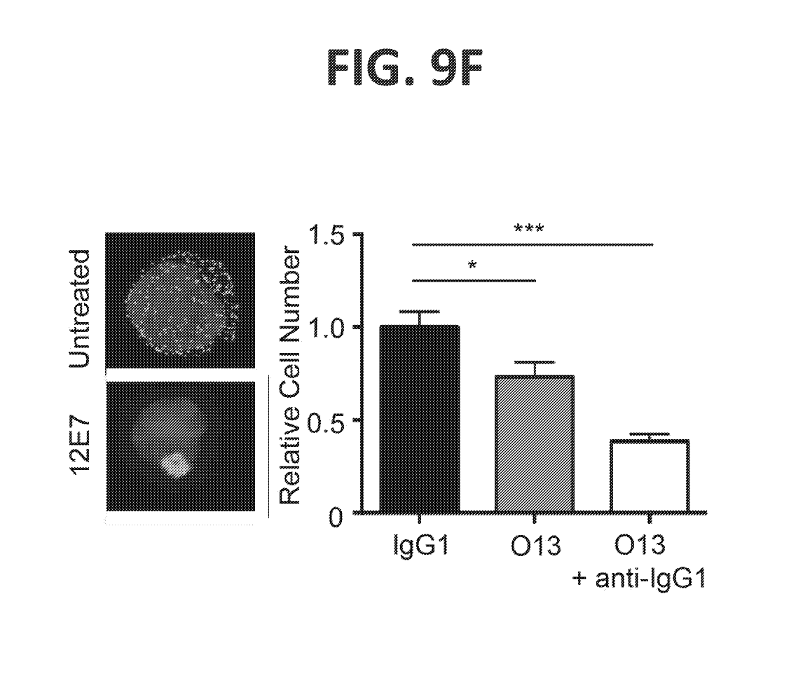

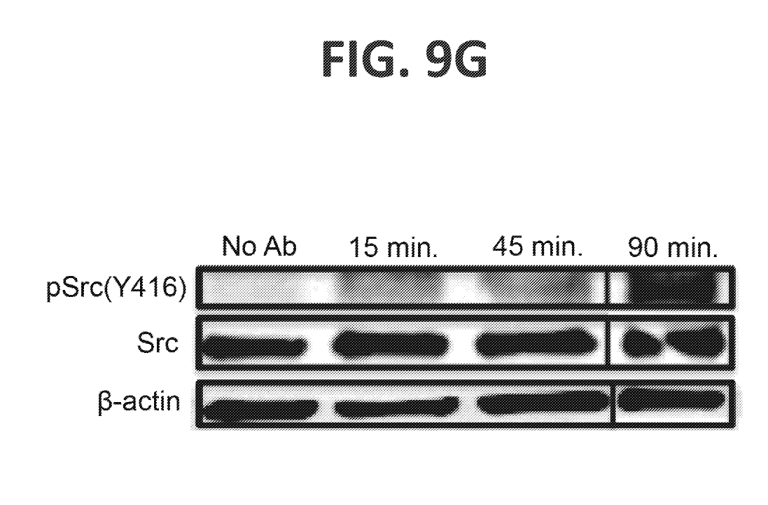

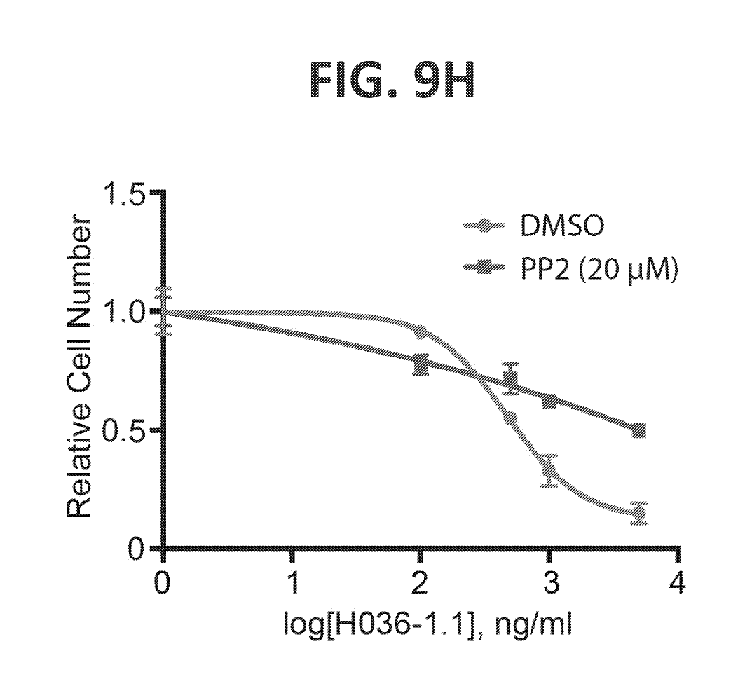

The ability of anti-CD99 mAbs to induce cell death in AML and MDS was tested. It was found that several anti-CD99 rnAb clones (12E7, O13, 11036-1.1) were cytotoxic to primary AML blasts and MDS CD34 cells (FIG. 9A), as well as 11 out of 15 AML cell lines (FIG. 9B) in vitro. Induction of apoptosis (FIG. 9C) in MOLM13 cells incubated with anti-CD99 inAb at 5 ng/mL showed an increase in activated caspase over time and occurred in the absence of immune effector cells or complement, suggesting that this is a direct cytotoxic effect. Apoptosis was induced independent of cell cycle status (data not shown) and could be inhibited with the pan-caspase anti.-CD99 antibody QVD (data not shown).

Although CD99 is expressed on normal HSCs (FIG. 6A) and endothelial cells (data not shown) at low and intermediate levels, respectively, anti-CD99 mAbs have very modest effects on these cell types in vitro, indicating a potentially wide therapeutic window (FIG. 91)). Ex vivo treatment of primary AML cells with anti-CD99 mAb (H036-1.1, 20 l.teml) prior to xenotransplantation into sublethally irradiated NSG mice led to a. significant decrease in engraftment as measured in the PB at eight weeks (20.5% vs. 67.4%, p=0.0(16, FIG. 9E). These experiments suggest that ey,7 vivo treatment of autologous bone marrow grafts from AML patients with anti-CD99 antibodies would be beneficial in that it would help deplete the grafts of disease-initiating cells, reducing the risk of relapse after transplant.

Cytotoxic anti-CD99 mAbs induced marked cell surface capping of CD99, and addition of secondary crosslinking antibodies to IgG isotype anti-CD99 mAbs (e.g., clone O13) recapitulates this capping effect and enhances cytotoxicity, suggesting that muitimerization of CD99 on the cell surface is key for inducing cell death (FIG. 9F). Cytotoxic anti-CD99 antibodies (e.g., 12E7) also induced cell surface aggregation and clustering of CD99, while non-cytotoxic anti-CD99 antibodies (e.g., 3B2) did not induce any appreciable redistribution of CD99 surface expression.

CD99 has been described to physically associate with and repress the activity of Src-family kinases (SF⁻Ks) in osteosarcoma cells. Scotlandi et al, Oncogene 26:6604-6618 (2007). It was confirmed that CD99 co-immunoprecipitates with SFKs in AML (data not shown), and that cytotoxic anti-CD99 mAbs induce robust SFK activation (FIG. 9G). Pharmacologic inhibition of SFKs with the small molecule anti-CD99 antibody PP2 significantly attenuates anti-CD99 mA.b induced cytotoxicity (FIG. 9H). Thus, by promoting dysregulated SFK. activation, anti-CD99 mAbs may promote cell death via oncogene induced apoptosis.

### Example 5

Anti-CD99 Monoclonal Antibody imAb) Eliminates AML Xenografis

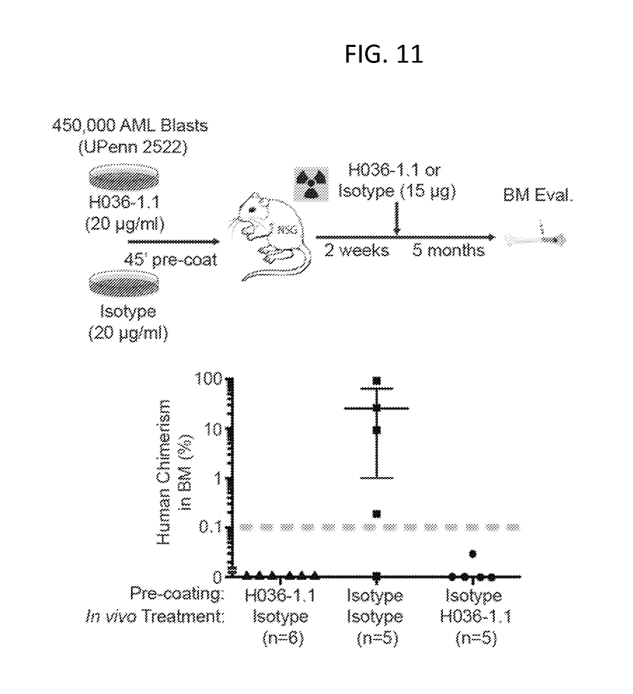

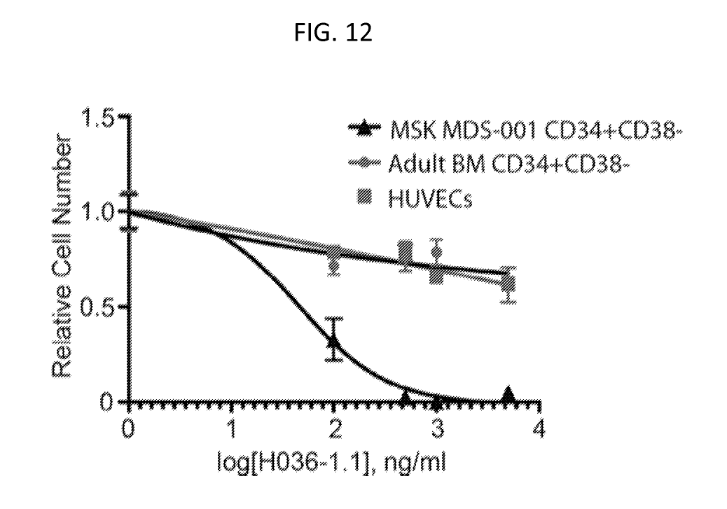

The ability of anti-CD99 mAbs to eliminate AML xenografts was tested. Combined ex vivo and in vivo H036-1.1 treatment of xenografted AML specimen UPenn 2522 in NSG mice was performed. A schematic of experimental protocol is shown in FIG, 11, upper panel. 450,000AML Blasts (UPenn 2522) were pre-coated with H036-1.1 anti-CD99 Mab (20 micrograms/mL) or isotype (20 micrograms/mL) for 45 minutes. The blasts were xenografted to sublethally irradiated NSG mice. After two weeks, mice were treated with H036-1.1 or isotype (15 micrograms). After 5 months, mice were sacrificed and bone marrow (BM) was evaluated. As shown in FIG. 11, lower panel, evaluation of mouse bone marrow (BM) for human chimerism after 5 months resulted in 0/6 animals that received blasts pre-coated with H036-1.1 and 0/5 animals treated in vivo with H036-1,1 exhibited >0.1% human engraftment in the BM (threshold demarcated with dotted gray line), while 4/5 control animals engrafted. Error bars represent +SD, As illustrated in FIG. 11, either pre-coating of AML blasts prior to xenograft or in vivo treatment of xenografted mice with an anti-CD99 MAb eliminated AML xenografts, while 4/5 control animals engrafted leukemia. The data demonstrate that either ex vivo pre-treatment of blasts or in vivo treatment of established grafts with H036-LI anti-CD99 MAb results in elimination of AML xenografts in NSG mice.

### Example 6

De novo Antibody Generation, Humanization, and Validation

Generation of de novo anti-CD99 inA.bs was performed by standard means. Mice were immunized with purified recombinant CD99 through a commercial vendor (Thermo Fisher) through conventional means. Screening of 85 candidate lead anti-CD99 mAbs was performed based on in vitro potency and efficacy. Hyhridorna supernatants containing Abs were evaluated compared to ligG Isotype negative control. Relative MOLM13 cell number after 72 hour incubation with hybridoma supernatants was performed. Three supernatants were identified that significantly decreased relative cell number. One candidate, supernatant 13, was particularly effective to reduce relative cell number. MOLM13 cells exposed to Supernatant 13 also exhibited decreased viability by PI staining and increased SSC, compared to IgG Isotype treated MOLM13 cells (data not shown). Future evaluation of candidates will include the criteria of exhibiting equivalent or greater in vitro potency as compared to the 12E7/11036-1.1/O13 MAbs. Additional characterization of the leads will be performed to ensure that the proapoptotic activity does not elicit killing in human endothelial cell lines and HSCs. Lead pro-apoptotic Ab's will also be evaluated in vitro for ADCC/CDC activity using human AML cell lines, primary AML cells, and LSCs. Affinity optimization of lead candidate Ab's to increase potency and efficacy, and in vivo validation of lead humanized mAb candidates will be performed.

Taken individually or together, these studies established CD99 as a promising prognostic marker and therapeutic target that is enriched on disease initiating cells in AML and MDS. Moreover, dysregulation of SFK activity by anti-CD99 niAbs represents a novel therapeutic vulnerability in AML and MDS that might be exploited using other modulators of this pathway. By directly inducing apoptosis and/or enhancing chemosensitivity, CD99 directed therapies may allow for the eradication of disease stem cells in ANI and MDS leading to durable remissions.

What is claimed is:

1. A method for identifying in a patient having acute myeloid leukemia (AML) and/or a myelodysplastic syndrome (MDS), the susceptibility of said patient to treatment with an anti-CD99 antibody, said method comprising:
a. quantifying the level of a CD99 mRNA in a cell from said patient by amplifying RNA in said cell with a primer pair that is specific for a CD99 gene sequence,
b. quantifying the level of a CD99 raRNA in a control cell from a healthy human subject by amplifying RNA in said cell with a. primer pair that is specific for a CD99 gene sequence, and
c. comparing the level of said CD99 raRNA in said cell from said patient with the level of said CD99 mRNA in said cell from said healthy human subject;
wherein an level of said CD99 niRNA in said patient cell as compared to said CD99 mRNA in said healthy human subject cell indicates the susceptibility of said patient to treatment with an anti-CD99 antibody;
wherein said anti-CD99 antibody:
(i) binds to the extracellular domain of CD99 on the surface of a CD99′ AML and/or MDS cell; and
(ii) promotes one or more of aggregation, clustering, and capping of said antibody bound CD99 on the surface of said AML and/or MDS cell;
said antibody inducing cell death in said AML and/or MDS cell when bound to CD99 on the surface of said AML and/or MDS cell.

2. The method of claim 1 wherein said primer pair comprises a forward primer and a reverse primer, wherein said forward primer hybridizes toward the 5′ end of said CD99 mRNA and wherein said reverse primer hybridizes toward the 3′ end of said CD99 tuRNA.

3. The method of claim 2 wherein said CD99 mRNA comprises a nucleotide sequence selected from the group consisting of SEQ ID NO: 1, SEQ ID NO: 3, and SEQ ID NO: 5.

4. The method of claim 1 wherein said cell is selected from the group consisting of a primary AML blast cell, a leukemic stein cell (LSC), a primary MDS blast cell, and an MDS heinatopoietic stem cell (HSC).

5. The method of claim 1 wherein said antibody binds to an epitope on CD99 comprising the amino acid sequence selected from the group consisting of DGEN (SEQ ID NO: 7), DAVVDGEND (SEQ ID NO: 10), AVVDGEN (SEQ ID NO: 11), and DDPRPPNPPK (SEQ ID NO: 12).

6. The method of claim 5 wherein said antibody is an IgG monoclonal antibody.

7. The method of claim 6 wherein said IgG monoclonal antibody is a murine antibody selected from the group consisting of 12E7 and O13.

8. The method of claim 1 Wherein said antibody is an IgM.

9. The method of claim 8 wherein said antibody is IgM monoclonal antibody H036-1.1.

10. The method of claim 1 wherein said antibody is conjugated to a second antibody.

11. The method. of claim 10 wherein said second antibody binds to the extracellular domain of CD99.

12. The method of claim 11 wherein said second antibody binds to an epitope on the extracellular domain of CD99 that comprises an amino acid sequence selected from the group consisting of DGEN (SEQ ID NO: 7), DAVVDGEND (SEQ ID NO: 10), AVVDGEN (SEQ ID NO: 11), and DDPRPPNPPK (SEQ ID NO: 12).

13. The method of claim 11 wherein said second antibody binds to an epitope on the extracellular domain of CD99 that does not comprise an amino acid sequence selected from the group consisting of DGEN (SEQ ID NO: 7), DAVVDGEND (SEQ ID NO: 10), AVVDGEN (SEQ ID NO: 11), and DDPRPPNPPK (SEQ ID NO: 12).

14. A method for inducing cell death in a CD99⁺ cell that is associated with acute myeloid leukemia (AML) and/or a myelodysplastic syndrome (MDS), said method comprising: contacting a CD99+ AML and/or MDS cell with an anti-CD99 antibody, wherein said antibody:
binds to the extracellular domain of CD99 on the surface of said CD99⁺ AML and/or MDS cell, wherein said antibody induces cell death in said AML and/or MDS cell
when bound to CD99 on the surface of said AML and/or MDS cell.

15. The method of claim 14 wherein said antibody promotes aggregation, clustering, and/or capping of said antibody bound CD99 on the surface of said AML and/or MDS cell.

16. A method for the treatment of a patient afflicted with an acute myeloid leukemia (AML) and/or a myelodysplastic syndrome (MDS) that is associated with a cell that exhibits an elevated level of CD99, said method comprising: administering to said patient a composition comprising an anti-CD99 antibody, wherein said antibody:
a. binds to the extracellular domain of CD99 on the surface of said CD99⁺ AML and/or MDS cell; and
b. promotes aggregation, clustering, and/or capping of said antibody bound CD99 on the surface of said AML and/or MDS cell:
said antibody inducing cell death in said AML and/or MDS cell when bound to CD99 on the surface of said AML and/or MDS cell.

17. A composition for the treatment of a patient afflicted with an acute myeloid leukemia (AML) and/or a myelodysplastic syndrome (MDS) that is associated with a cell that exhibits an elevated level of CD99, said composition comprising:
a. a first anti-CD99 antibody, wherein said first antibody:
i. binds to the extraceilular domain of CD99 on the surface of said CD99 AML and/or MDS cell;
ii. promotes aggregation, clustering, and/or capping of said antibody bound CD99 on the surface of said AML and/or MDS cell; and
iii. induces cell death in said AML and/or MDS cell when bound to CD99 on the surface of said AML and/or MDS cell, and
b. a second anti-CD99 antibody, wherein said second antibody inhibits tansendothelial migration of said CD99 AML and/or MDS cell.

18. The composition of claim 16 wherein said second antibody is selected from the group consisting of 12E7, HEC2, DN16, and H036.

19. A method for inhibiting the proliferation of a. CD99+AML and/or MDS cell, said method comprising contacting said CD99+ AML and/or MDS cell with an anti-CD99 antibody, wherein said antibody:
a. binds to the extracellular domain of CD99 on the surface of said CD99⁺ AML and/or MDS cell; and
b. promotes aggregation, clustering, and/or capping of said antibody bound CD99 on the surface of said AML and/or MDS cell;
said antibody inducing cell death in said AML and/or MDS cell when bound to CD99 on the surface of said AML and/or MDS cell.

20. The method of claim 18 wherein said anti-CD99 antibody binds to said extracellular domain of CD99 with a K. of from about 100 nM to about 10 μM or from about 250 nM to about 5 μM or from about 500 nM to about 1 μM.

21. The method of claim 18 further comprising contacting said CD99+ AML and/or MDS cell with a compound selected from the group consisting of daunorubicin, idarubicin, cytarabine, azacytidine, and decitabine.

22. The method of claim 20 wherein said compound is added simultaneously with said anti-CD99 antibody.

23. The method of claim 20 wherein said compound and said anti-CD99 antibody are added sequentially.

24. A method for treating AML and/or MDS in a patient, comprising: administering to said patient an anti-CD99 antibody, in an amount effective to induce cytotoxicity of said AML and/or MDS cell; said patient being afflicted with an AML and/or MDS that exhibits an elevated level of CD99 expression as compared to the level of CD99 expression in a control non-AML and/or non-MDS cell.

25. The method of claim 24 further comprising administering to said AML and/or MDS patient a chemotherapeutically effective amount of a compound selected from the group consisting of daunorubicin, idambicin, eytarabine, azacytidine, and decitabine.

26. The method of claim 24 wherein said compound is administered simultaneously with said anti-CD99 antibody.

27. The method of claim 24 wherein said compound is administered sequentially to said anti-CD99 antibody.

28. The method of claim 23 further comprising administering to said NW. andlor MDS patient a mobilizing agent.

29. The method of claim 27 wherein said mobilizing agent is plerixafor or CSF or a combination thereof.

30. The method of claim 27 wherein said mobilizing agent is administered to said AML and/or MDS patient prior to administering said anti-CD99 antibody.

31. A method for treating AML and/or MDS in a patient previously identified as afflicted by an AML and/or MDS cell exhibiting an elevated level of expression of a CD99 gene: administering to said patient an anti-CD99 antibody in an amount effective to induce cytotoxicity in said AML and/or MDS cell.

32. The method of claim 31 further comprising administering to said AML and/or MDS patient a compound selected from the group consisting of daunorubicin, idarubicin, cytarabine. azacytidine, and decitabine.

33. The method of claim 31 wherein said compound is administered simultaneously with said anti-CD99 antibody.

34. The method of claim 31 wherein said compound is administered subsequent to said anti-CD99 antibody.

35. The method of claim 30 further comprising administering to said AML and/or MDS patient a mobilizing agent.

36. The method of claim 34 wherein said mobilizing agent is plurixefore or G-CSF or a combination thereof.

37. The method of claim 34 wherein said mobilizing agent is administered to said AML and or MDS patient prior to administering said anti-CD99 antibody.

38. A method for determining the susceptibility of an AML and/or MDS patient to treatment with an anti-CD99 antibody, said method comprising:
a. determining in a cell from said AML and/or MDS patient the level of expression of a CD99 gene;
b. determining in a cell from a non-leukemia donor control cell the level of expression of said CD99 gene;
c. comparing the level of said CD99 gene in the patient cell to the corresponding level of gene expression in the control cell;
wherein a level of said CD99 gene expression in said AML and/or MDS cell that is at least about two-fold greater than the corresponding level of gene expression in said control cell is predictive of the therapeutic efficacy of said anti-CD99 antibody, wherein said anti-CD99 antibody can promote at least one of the aggregation, clustering or capping of said CD99 when bound CD99 on the surface of said AML and/or MDS cell.

39. A method for predicting the susceptibility of an AML and/or an MDS patient to treatment with an anti-CD99 antibody, said method comprising: testing an AML and/or MDS patient cell for the elevated expression of CD99, wherein said elevated expression of CD99 is predictive of the therapeutic efficacy of an anti-CD99 antibody that promotes at least one of the aggregation, clustering or capping of CD99 when bound to said CD99 on the surface of said AML and or MDS cell.

40. The method of claim 38 further comprising testing said ANIL and/or MDS patient cell for c-kit gene expression, wherein the absence or a low level of c-kit gene expression is predictive of the susceptibility of an AML and/or an MDS patient to treatment with an anti-CD99 antibody.

41. The method of claim 38 further comprising testing said ANIL and/or MDS patient cell for the presence of a BCR-ABL translocation, wherein the presence of BCR-ABL is predictive of the susceptibility of an AML andlor MDS patient to treatment with an anti-CD99 antibody.

42. The method of claim 38 further comprising testing said ANIL and/or MDS patient cell for the presence of mutated JAK2, wherein the presence of constitutively activating JAK2 mutations is predictive of the susceptibility of an AML andlor an MDS patient to treatment with an anti-CD99 antibody.

43. A method for predicting the susceptibility of an AML and/or on MDS patient to treatment with an anti-CD99 antibody, said method comprising: testing an AML and/or MDS patient cell for the elevated expression of CD99, wherein said elevated expression of CD99 is predictive of the therapeutic efficacy of an anti-CD99 antibody is cytotoxic when bound to said CD99 on the surface of said AM⁻L and or MDS cell.

44. A method for predicting the susceptibility of an AML and/or an MDS patient to treatment with an anti-CD99 antibody, said method comprising: testing an AML andlor MDS patient cell for the elevated expression of CD99, wherein said elevated expression of CD99 is predictive of the therapeutic efficacy of an anti-CD99 antibody induces cell death when bound to said CD99 on the surface of said ANIL and or MDS cell.

45. A method for generating a candidate anti-CD99 antibody for the treatment of acute myeloid leukemia and/or a myelodysplastic syndrome, said method comprising:
a. generating an antibody that binds to the extracellular domain of CD99
b. testing said anti-CD99 antibody for
i. antibody-mediated aggregation, clustering, andlor capping of AML and/or MDS cell-surface expressed CD99 and
ii. antibody-mediated induction of cell death of said AML and/or MDS cell-surface expressed CD99,
wherein an anti-CD99 antibody that mediates said aggregation, clustering, and/or capping of CD99 and induces said cell death is a candidate anti-CD99 antibody for the treatment of acute myeloid leukemia andlor a myelodysplastic syndrome.

46. A method for determining whether an anti-CD99 antibody is a candidate for use in the treatment of an acute myeloid leukemia andlor myelodysplastic syndrome in a patient wherein Aml or MDs cells respectively express elevated levels of CD99, said method comprising:
a. providing an anti-CD99 antibody;
b. subjecting said anti-CD99 antibody to one of more tests for determining whether it results in for
antibody-mediated aggregation, clustering, and/or capping of AML and/or MDS cell-surface expressed CD99 and
wherein if said aggregation, clustering and/or capping occurs, concluding that the antibody is cytotoxic to CD99+ AML or MDS cells and as such a candidate for the treatment of acute myeloid leukemia and/or a myeiodysplastic syndrome; alternatively, if said aggregation, clustering andlor capping does not occur concluding that the antibody is not a candidate for said treatment.

47. A method for the treatment of an AML and/or MDS patient exhibiting elevated levels of CD99 on the surface of AML or MDS cells, said method comprising:
administering to said patient one or more anti-CD99 antibodies, a composition or formulation comprising one or more anti-CD99 antibodies, and/or a composition or formulation comprising one or more anti-CD99 antibodies in combination with one or more other agent that is effective in he treatment of AML andior MDS; wherein anti-CD99 antibody induces cell death when bound to the surface of said cell.

48. A method for identifying in an ANIL andior MDS patient_{;} the susceptibility of said patient to treatment with an anti-CD99 antibody, said method comprising:
a. quantifying the level of a CD99 MRNA in a cell from said patient,
b. quantifying the level of a non-CD99 control RNA in said cell from said patient, and
c. comparing the level of said CD99 aiRNA in said cell with the level of said control RNA in said cell thereby obtaining a ratio of gene expression for a CD99 gene and a control gene,
wherein a ratio of gene expression for a CD99 gene and for a control gene in said cell that is greater than a pre-determined threshold ratio for a CD99 gene and for a control gene in a non-AML and/or non-MDS cell indicates the susceptibility of said patient to treatment with an anti-CD99 antibody that induces apoptosis when bound to CD99 on the surface of an AMD and/or MDS cell.

49. The method of claim 47 wherein said control mRNA is selected from the group consisting ofβ-actin, GAPDEI, and cyclophilin.

50. A method for identifying in a patient having acute myeloid leukemia (AML) and/or a myelodysplastic syndrome (ryfDS), the susceptibility of said patient to treatment with an anti-CD99 antibody, said method comprising;
a. quantifying the level of a CD99 tfiRNA in a cell from said patient by amplifying RNA in said cell with a primer pair that is specific for a CD99 gene sequence,
b. quantifying the level of a CD99 mRNA in a control cell from a healthy human subject by amplifying RNA in said cell with a primer pair that is specific for a CD99 gene sequence, and
c. comparing the level of said CD99 mRNA in said cell from said patient with the level of said CD99 mRNA in said cell from said healthy human subject;
wherein an level of said CD99 nRNA in said patient cell as compared to said CD99 mRNA in said healthy human subject cell indicates the susceptibility of said patient to treatment with an anti-CD99 antibody;
wherein said anti-CD99 antibody:
induces cell death in said AML and/or MDS cell when bound to CD99 on the surface of said AML and/or MDS cell.
